(19) United States
(12) Patent Application Publication
(10) Pub. No.: US 2016/0369327 A1
(43) Pub. Date: Dec. 22, 2016

(54) FLUOROPHORE-BASED OLIGONUCLEOTIDE PROBES WITH A UNIVERSAL ELEMENT

(71) Applicants: LGC LIMITED, Middlesex (GB); UNIVERSITY OF SOUTHAMPTON, Hampshire (GB)

(72) Inventors: David French, Middlesex (GB); Paul Debenham, Middlesex (GB); Rebecca Howard, Middlesex (GB); Tom Brown, Oxford (GB); James Richardson, Near Saffron Walden (GB)

(21) Appl. No.: 14/898,953

(22) PCT Filed: Jun. 18, 2014
(86) PCT No.: PCT/GB2014/000240

§ 371 Date: Dec. 16, 2015

(30) Foreign Application Priority Data
Jun. 18, 2013 (GB) 1310823.8

(51) Int. Cl. C12Q 1/68

(52) U.S. Cl. CPC C12Q 1/6832; C12Q 1/6818; C12Q 1/6876; C12Q 2600/156; C12Q 2600/16

(57) ABSTRACT

There is provided a method of detecting the presence of a target polynucleotide and/or sequence variations within the target polynucleotide using a probe system comprising two independent partner oligonucleotide components wherein the first oligonucleotide has a first and second section, wherein the first section comprises a nucleotide sequence that is labelled with at least one visually detectable label and is not capable of hybridising to the nucleotide sequence of the target polynucleotide; and wherein the second section comprises a nucleotide sequence that is capable of hybridising to a portion of the sequence of the target polynucleotide; and the second oligonucleotide has a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to. There are also provided alternative methods using additional oligonucleotides and probes for use in such methods.

The present invention relates to oligonucleotides, and in particular their uses as probes in the detection of hybridisation events and nucleic acid detection and/or discrimination using fluorescence.

Probe technologies based on fluorophore-labelled oligonucleotides are long-established, especially with the fluorophore as an addition at the 5′ phosphate terminal. Such terminal labelling is inexpensive and hence the development and application of such probes is wide-spread across molecular biology applications. In contrast an advantageous design of probes, in which multiple fluorophores can be attached to the nucleoside bases or sugars within the oligonucleotides sequence, such as HyBeacon® probes (French et al. (2001), WO 01/73118, WO 07/010268), have been less widely applied because of the inherently more expensive nature of such internal fluorophore additions.

The long established field of using labelled nucleic acid oligonucleotides as a reporter for the presence of a target nucleic acid sequence (DNA or RNA) utilises the direct nucleotide sequence complementarity of an oligonucleotide based probe with its target nucleic acid sequence and its unique match achieved by sufficient shared sequence compared to the genomic background.

The deliberate use of either partial sequence complementarity or the use of additional oligonucleotides providing intermediary hybridisation events between the probe and its target is unnecessary unless achieving a particular additional molecular end point alongside the probe-target interaction is desired.

In the example of a fluorescent oligonucleotide probe, the important incremental benefit of the direct sequence complementarity found in conventional probe-target hybridised pairing is that any sequence variation in the target, such as a single nucleotide polymorphism (SNP), is directly disruptive to that hybridisation and hence the fluorescent output of the probe. It can therefore be presumed that intermediary oligonucleotides would interfere with, or reduce any such direct detection of a SNP in the target sequence. Further the use of additional oligonucleotides in the probing complex would normally appear an unnecessary cost.

There is herein described a novel variation in the design of internally labelled probes which aligns with a two-phase oligonucleotide synthesis procedure. This two-phase synthesis route still uses standard phosphoramidite-linked fluorophores, but achieves significantly reduced synthesis costs compared to the conventional process for such probes.

The presently described invention describes the use of additional oligonucleotides as an enabling methodology to reduce the cost of probe-based analysis, especially where multiple or expensive fluorophore chemistries are involved, without detrimental effect to the detection of SNPs and other target sequence variations.

The key design feature of the new nucleic acid based probes generated (hereinafter called Junction Probes) is that the Junction Probe is constructed in two parts; a first ‘universal element’ part of the sequence containing all the fluorophore signalling chemistry bound to internal nucleotides and the remainder part being sequence that is chosen to be complementary to the target sequence of interest.

The universal element by definition is not complementary to the target sequence and would not bind to the target DNA. However the nature of the internal fluorophore labelling as observed in other fluorescent probes (e.g. HyBeacon® probes) is that the fluorophores have significantly enhanced fluorescence only when hybridised to a complementary sequence. The complementary sequence of existing probes would normally be the target sequence, as the purpose of probe analysis is reporting both the presence of the target sequence in the analysis as well as any target sequence variation (such as single nucleotide polymorphisms (SNPs)).

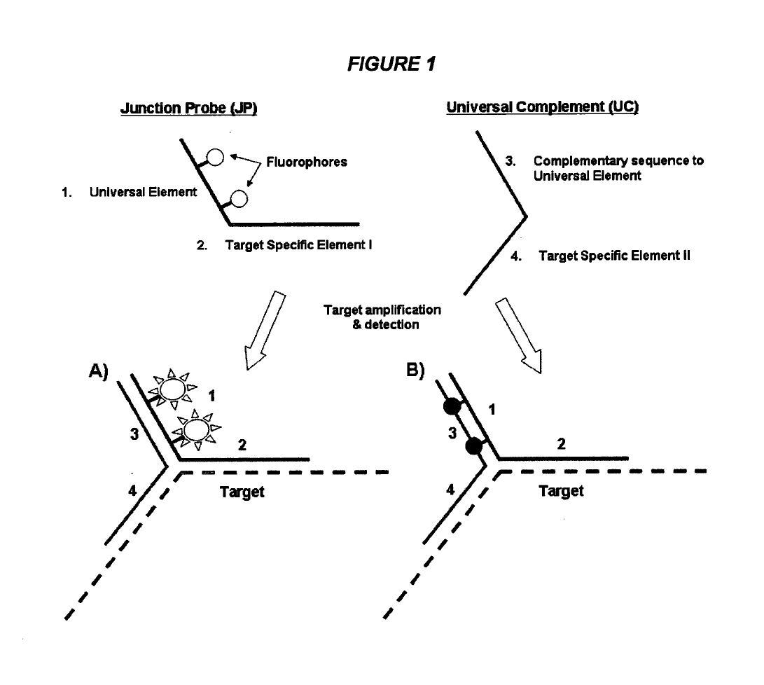

A requirement of the new probe design is therefore that a second oligonucleotide (Universal Complement: UC) is used and it both hybridises to the target sequence as well as being complementary to the universal element sequence of the probe. As a consequence the new invention described herein uses a three-way hybridisation event which we have named a ‘tribrid’ (triple hybridisation) and is schematically drawn in FIG. 1.

The use of the additional oligonucleotide partner (i.e. the universal complement) to partner the junction probe does add additional cost to the design and use of the universal probe. However, this increased cost in additional oligonucleotides is relatively minimal compared to the cost savings achieved by the use of a single universal element (prepared using the two-phase oligonucleotide synthesis procedure) as part of multiple junction probes of different target specificity.

The herein described probe system represents a new concept in probe structure that is also more efficient and easier to use than existing fluorescent oligonucleotide probes. The herein described probe system is of particular relevance where the oligonucleotides forming the probes are internally labelled (i.e. not at their 3′ or 5′ ends) with a visually detectable label (such as a fluorophore or dye). The formation of the tribrid successfully recreates the hybridisation necessary for the change in fluorescence in the ‘universal element’ arm of the tribrid at temperatures at which it would not in its own right be stably paired. The probe system also is capable of detecting the presence of SNPs in the target sequence falling within the other two hybrid arms, even in the UC oligo-target hybrid which is not contiguous with the probe sequence at all. Most importantly the additional utility of a probe system to clearly detect a target sequence polymorphism is in fact found to occur in this tribrid structure even when the target sequence variation is within the strand complementary to the universal complement oligonucleotide and there is no direct connection between the probe and target sequence at this point (e.g. FIG. 1).

One important aspect of the herein described internally-labelled probes is the observation that in certain designs the formation of the tribrid enhances fluorescence. This enhancement is seen by a positive melting curve peak. In addition the herein described internally-labelled probes when in the tribrid form may quench the fluorescence from the fluorophores relative to that observed from the probe in an unhybridised state. This quenching is associated with an inverse or negative melting curve. Whether an internally-labelled probe enhances fluorescence (positive melting peak) or quenches it (negative melting peak) depends on a number of factors, including the distance between the probe junction and the 5′-most visually detectable label (if the probe has been synthesised in the 5′ to 3′ direction, otherwise 3′-most visually detectable label) and the probe sequence, as will be described later. Such factors are readily determinable without undue experimentation by one of skill in the art.

For any particular target sequence, the current synthetic process for oligonucleotide probes is that each oligonucleotide is produced as a new sequence thereby consuming expensive pre-labelled nucleotides (e.g. fluorescent pre-labelling). Conventional probe synthesis has a minimum chemistry requirement in the 0.1 μM range which generates tens of thousands of copies of the oligo probe which is ideal for high usage assay preparation as the final costing per probe test is minimal. However for research studies in which many variations of probes are being investigated it will be common practice that the vast majority of the probe studies only involve a small number of experiments such that the majority of each probe synthesis is not required and is essentially wasted.

Oligonucleotide synthesis typically proceeds by the stepwise addition of phosphoramidite activated nucleotides or other phosphorus derivatives to a growing oligonucleotide chain on a cleavable solid support. This process continues until the complete oligonucleotide is assembled and is then cleaved from the solid support. Oligonucleotide synthesis is conventionally carried out in the 3′ to 5′ direction rather than the 5′ to 3′ direction due to the fact that the requisite monomers are easier to synthesise, but both directions of synthesis are possible. The process described below applies to both directions of oligonucleotide synthesis on solid support. It is a method by which large numbers of oligonucleotides can be synthesised in two or more stages (large scale followed by small scale) using the differing scales to achieve benefits in cost.

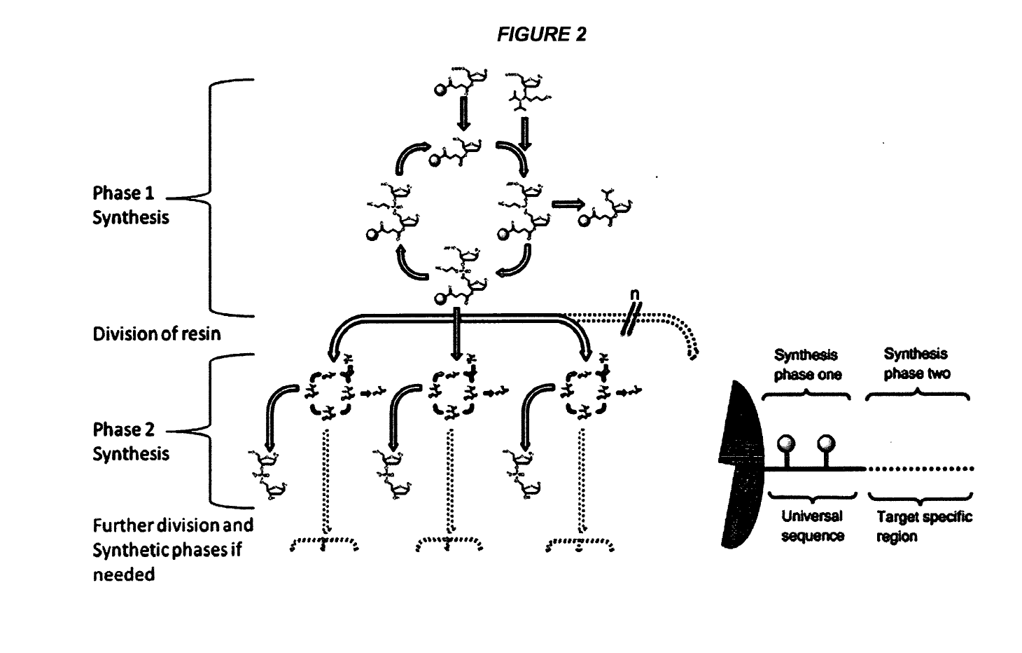

The synthesis of oligonucleotides on a large scale (e.g. 15 micromole or greater) allows highly efficient oligonucleotide synthesisers to be used with optimised synthesis cycling conditions and larger economies of scale resulting from more efficient use of reagents and solvents. The large scale synthesis of a universal portion of an oligonucleotide containing expensive modifications such as dye labelled nucleobases or protected aminoalkyl, alkyne or cycloalkyne-labelled nucleobases (either the 5′-portion or the 3′-portion of the oligonucleotide) is particularly cost-effective. The oligonucleotide that has been assembled in the initial large scale synthesis is left attached to the solid support, with all its nucleobase, phosphate and terminal hydroxyl protecting groups attached. The solid support is then subdivided into small portions and further oligonucleotide synthesis can be performed on each portion separately (FIG. 2). After assembly and prior to (or post) the subdivision stage the “large scale” oligonucleotide attached to the solid support can (if required) be modified by methods including but not limited to amide formation, click chemistry, Diels-Alder and maleimide labelling, giving rise to a number of potential modified end products. This method can be used to add fluorescent dyes, haptens, SERS labels or other reporter groups or ligands. Labelling the oligonucleotide whilst on the solid support allows anhydrous solvents to be used, increasing the life time of some labelling reagents (e.g. active esters, maleimides) during labelling, thereby improving the efficiency of the labelling reaction. Furthermore, using large scales and highly efficient large scale synthesisers such as the ÄKTA OligoPilot (GE Healthcare) with recirculating reagents in the labelling procedure facilitates efficient use of reagents.

Once the solid support has been divided and any labelling modification (if required) has been performed, the individual smaller quantities of solid support can be treated individually, and subjected to further rounds of oligonucleotide synthesis. Each individual batch can then have a different sequence assembled onto it (the variable target specific element) and this second phase of synthesis can be done on a variety of scales from 1.0 micromole down to 10 nanomole or smaller. Many different small scale DNA synthesisers can be used for this second phase of oligonucleotide synthesis (e.g. ABI 3400, ABI 3900, ABI 394, PerSeptive Expedite, Dr Oligo 96/193, Bioautomation MerMade). These subsequent smaller syntheses can then be deprotected using standard conditions giving small scale products at very low cost. This process is as efficient as standard single phase oligonucleotide synthesis, yielding high quality oligonucleotides. Oligonucleotide purification by gel-electrophoresis or HPLC is unnecessary for applications using HyBeacon® probes synthesised by this method because of high synthesis quality.

The methodology is particularly cost effective when large numbers of oligonucleotides are required for SNP panels and in areas of research where large numbers of modified oligonucleotides have to be used in screening for DNA or RNA targets. Furthermore, the initial large scale synthesis can be used as a resource to provide a resin-bound universal probe element library. Different members of the library can each have different coloured fluorophores. One advantage of this method is that the universal elements can be optimised before being admitted into the library. Thus the library can then be used to generate probes with a high probability of functioning efficiently. The present invention addresses this concern with a probe design that allows a synthesis method to be used that can reduce costs significantly, for example for internally labelled oligonucleotides probes such as HyBeacons®.

In an alternative synthesis approach, the universal element and target specific sequences are synthesised separately and combined using ‘click-chemistry’ methods (see later in this paragraph for references). The universal element sequence is synthesised on a large scale with a 5′-cyclooctyne derivative and individual target-specific oligonucleotides (with 3′-azide) are be made on a small scale. The universal element and individual target-specific probes are then clicked together in a ring-strain promoted alkyne-azide cycloaddition reaction (SPAAC reaction), simply by mixing the two oligonucleotides in aqueous buffer to provide potentially thousands of probes from a single universal element synthesis (Shelbourne et al. 2011, Shelbourne et al. 2012, Gerrard et al. 2012). Alternatively, the azide can be located on the universal element and the cycloalkyne can be on the target-specific probe. As an alternative to the SPAAC reaction, the CuAAC reaction (copper-catalysed alkyne-azide cycloaddition reaction) can be used (Kumar et al. 2007, El-Sagheer et al. 2009, El-Sagheer et al. 2011, El-Sagheer et al. 2012), in which the cycloalkyne is replaced by a terminal alkyne and the reaction is catalysed by Cu(I). Another alternative is to use the Diels-Alder reaction to ligate the two oligonucleotides in which one oligonucleotide is labelled with a diene and the other with a dienophile (El-Sagheer et al. 2011). The above click reactions between two oligonucleotides can be assisted by a complementary oligonucleotide splint to improve the efficiency and rate of the reaction, but this is not essential.

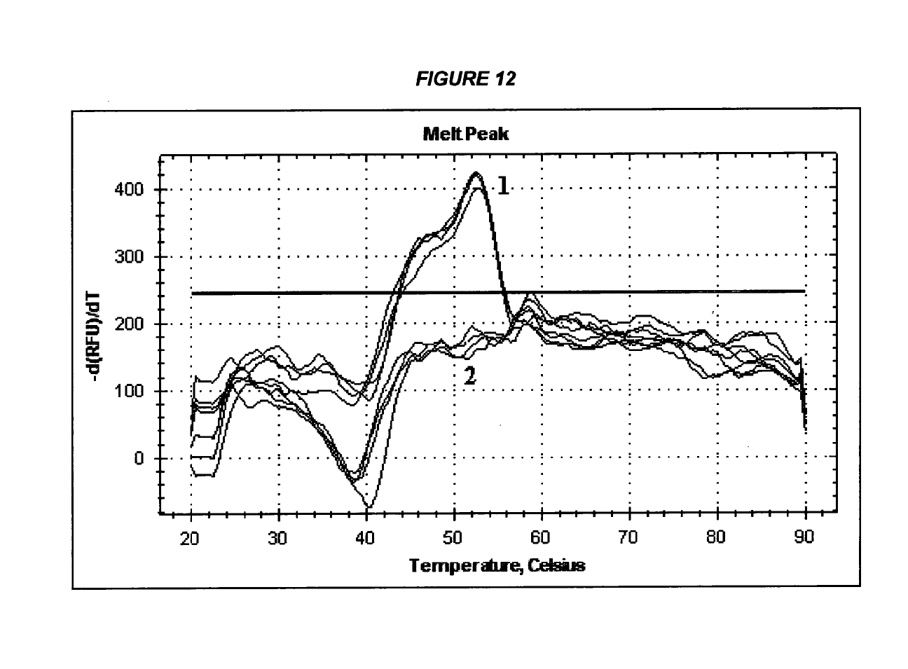

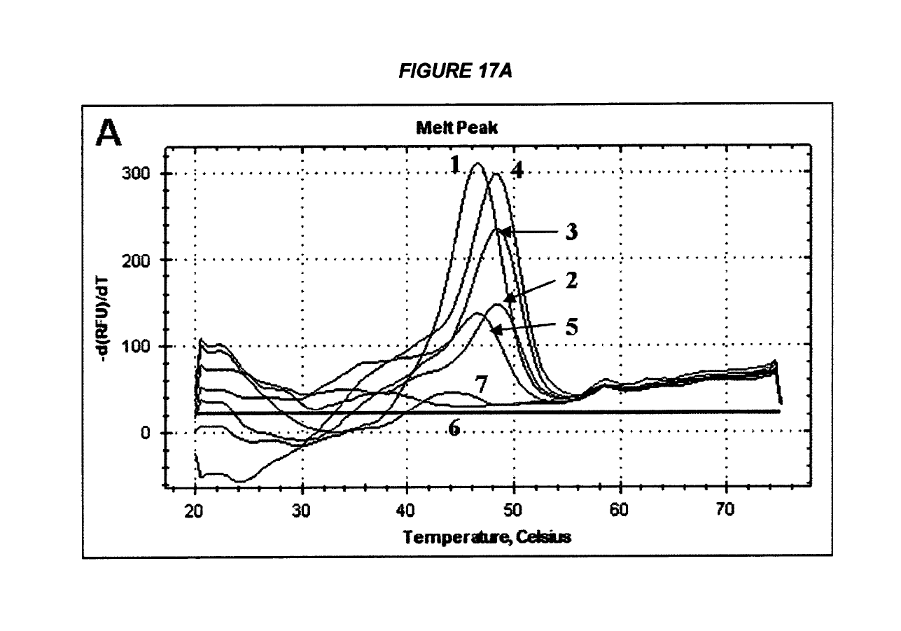

WO09/053679 is directed towards discriminating the number of repeating sequence elements in Short Tandem Repeats (STRs) using HyBeacon® probes. FIG. 12 and claim 38 of WO09/053679, for example, relate to an arrangement in which the HyBeacon® probe and a ‘blocker’ oligonucleotide have a junction (“clamp”) to anchor probe hybridisation against the target sequence. The HyBeacon® fluorophores are placed within the probe sequence so as to be in the part that hybridises with the target nucleic acid sequence, thereby leading to a change in fluorescence on hybridisation with the target nucleic acid sequence. FIG. 17 of WO09/053679 shows an alternative arrangement in which the probe and blocker oligonucleotides have terminal fluorophores forming a FRET pair or fluorophore:quencher pair when the clamp portions are hybridised. In each “clamp” arrangement of WO 09/053679 the clamp portions typically have 3 to 10 nucleotides, for example from 4 to 8, such as 6 to 8. The clamp portion typically contributes between 10° C. and 30° C. to thermal stability in order to ensure that the probe hybridises to the correct sequence and preventing slippage. The Tm of the clamp portion should not increase the Tm of the probe oligonucleotide to the extent that it prevents discrimination of similar length target sequences. The Tm for hybridisation between the probe and the target nucleic acid is typically between 40° C. and 70° C.

This is an example demonstrating that the use of an additional oligonucleotide hybridisation event can enhance detection of a specific target sequence. However, the probe design involved in the method of WO09/053679 requires internally labelled bases that are specifically positioned to be complementary to the target sequence. As this is a target specific design there no cost saving achieved by the use of the additional oligonucleotide intervention.

More recently, Hsin-Chih Yeh and colleagues have proposed a form of three-way oligonucleotide structure to activate their novel signalling nanoclusters of silver (US 2011/0212540 A1). In US 2011/0212540, the non-target oligonucleotide sections of the oligonucleotides interact to provide a non-hybridising interface for the silver atoms. The proximity of an oligonucleotide section with a preferred sequence motif can be designed to influence the nanocluster fluorescence. Only very limited hybridisation (˜4 base pairs) can be tolerated between this non-target sections without detrimental increase to the fluorescent background. No consideration is presented with respect to whether such a structure can, or cannot detect sequence variation in the target. The particular design of US 2011/0212540 has a number of features that are significantly different from aspects of the tribrid structure of the present invention, which include:
(i) The sequence element containing the silver nanocluster label does not participate in a hybridisation event but requires a proximity positioning of a DNA strand to achieve colour emission enhancement [0049, line 13/14]: whereas in complete contrast the tribrid structure is only effective in fluorescence enhancement when the labelled sequence is actively engaged in hybridisation
(ii) The sequence containing the nanoclusters should not participate in hybridisation over more than more 4 bases due to detrimental background fluorescence increasing as the length of hybrid increases [0068, lines 8-11]; in contrast to the junction probe configuration than can hybridise along the full length of the label contain sequence (to ˜20 bases) with no detriment to the fluorescent emission (default observation unless UC designed to quench the fluorescence).
(iii) The strand proving proximal enhancement provides differing colour enhancement to the silver nanoclusters fluorescence depending on its sequence. If it contain at least one guanine base then a red fluorescence is detected [0049, lines 13/14], while if thymine bases are present a green fluorescence is observed [0034] and with adenine present no colour fluorescent enhancement is observed [0069 lines 11-13]; whereas the sequence of the UC oligonucleotide does not influence fluorescent colour emission with the tribrid structure.
(iv) The strand containing the attachment of the silver nanoclusters has a preferred sequence motif (C₃NNNNNC₄) where N=either a thymine or adenine base [0066, lines 21-23]; whereas the tribrid probe sequence with internal fluorophores has no sequence specification (just some simple fluorophore location parameters)
(v) The hybrid structure is formed in a buffer of 20 mM pH 6.6 sodium phosphate and requires at least a 50 minute cool down step following a 1 minute 95° C. treatment [0043, lines 25-29]; whereas the tribrid is compatible with PCR and other amplification buffers, requires the briefest of exposures to 90+° C. degrees and is formed instantly within a rapid cool down to room temperature.
(vi) No mention is made of the ability to detect target sequence variation along the length of the probe sequence or proximity sequence with respect to the ability to detect sequence variations; whereas the tribrid has been established to detect such sequence variation in both the sequence complementary to the junction probe and the sequence complementary to the UC oligonucleotide.

A realtime analysis method called NuPCR™ (Illumina) uses two “PartZyme™ oligos” to allow a terminally labelled probe to detect the presence of a target nucleotide sequence. The NuPCR™ system requires cleavage of the probe by the PartZyme™ oligos in order to allow a terminal fluorescent label to be detected by a real-time PCR instrument.

The NuPCR™ method uses “NuZymes™” that recognize and assemble on target DNA sequences forming a catalytic complex. The enzymatic activity of the NuZyme™ cleaves a fluorescently-labelled universal substrate, producing a signal that can be detected by a real-time PCR instrument. NuZymes™ are composed of two oligonucleotides that are partial enzymes or PartZyme™, each containing part of the catalytic core sequence of the NuZyme™, flanked by a substrate arm sequence and a sensor arm sequence. PartZymes™ contain no catalytic activity individually or in the absence of the target sequence. The target sequence serves to bring the two PartZymes™ in close proximity, which facilitates the formation of the catalytically active NuZymeT™.

(http://www.illumina.com/documents//products/brochures/brochure_nuper.pdf)

The present invention differs from the NuPCR approach in that it does not require catalysis of the probe oligonucleotide(s) in order for the label to be detected, and that in some embodiments, the labels are internal to the oligonucleotides.

Examples of other multiple component detection systems are described in WO 99/42616, WO 2011/063388, WO2006/097506, EP0552931, WO 02/002817, WO 2010/017246, WO 01/38570 and WO 99/37806.

WO 99/42616 describes a two component system that is used at a single temperature whereby both oligonucleotides are labelled and both labels are required for signal generation.

WO 2011/063388 describes a two component system that forms an endonuclease site on hybridisation. The oligonucleotides are labelled with both a reporter and a quencher where by digestion by an endonuclease releases the reporter from the quencher to form a signal.

WO2006/097506 also describes a multi component system whereby a quencher and fluorophore pair are used, with the quencher being required to prevent signal before target binding. This method however requires a single read temperature and is unable to conduct SNP detection without the inclusion of multiple probes with different dyes.

EP 0552931 describes multicomponent systems of 3 or 4 strand complexes and describes Tm measurement at a single incubation temperature with a series of measurements being taken (as opposed to the traditional melting or annealing curve analysis). The system can potentially (but with some difficulty) differentiate between multiple sequence variants by using different probes for each target.

WO 02/002817 provides labelled oligonucleotides where the complex formation is analysed using electrophoresis (i.e. at a single temperature).

WO 2010/017246 describes an analysis method using cleavage of a restriction site created by formation of the oligonucleotide complex. Cleavage is conducted at a single reaction temperature

WO 01/38570 describes a multiple oligonucleotide system whereby the probe is cleaved on formation of the complex in the presence of target DNA, with cleavage occurring at a single reaction temperature.

WO 99/37806 describes a 3-strand) complex in the presence of target DNA, where one probe is extended using the other probe as template with analysis being conducted at a single read temperature to detect extension of the first probe.

The present inventors have found that a useful hybridisation probing/detection/discrimination system can make use of a ‘universal’ section of a probe (which may be termed a “a multiplex of three (or more e.g. four) oligonucleotides; also referred to herein as the junction probe”) that can be created containing internal fluorophore labelling which could be prepared in high amount to gain economies of scale. In one design option, a subsequent second phase of probe synthesis customises the remaining probe sequence to match the target sequence, for example as set out in Example 17 below. We describe further below the ‘tribrid’ probe configuration that takes advantage of the multi- (for example two-) phase probe synthesis. Other design options use a universal probe oligonucleotide alongside two (or potentially more) oligonucleotides each with a target nucleic acid-specific portion and universal complement portion that hybridise to the target nucleic acid and universal probe oligonucleotide to form, for example, a “cruciform” four-molecule configuration.

The hybridisation probing event within the present invention is the formation of a multiplex of three or more, for example three or four, nucleic acid molecules; two oligonucleotides each with a target-specific portion and a further portion; optionally a further oligonucleotide(s), for example able to hybridise to both said further portions; and a third nucleotide sequence, that of the target sequence. The co-operation of three oligonucleotide elements in a hybridisation probing event has been used widely over many years but only as a combination of two adjacent or closely located pairs of oligonucleotides with the target sequence rather than in a tribrid formulation. This can occur in FRET probe configurations (Didenko, 2001) in which the excitation of a label attached to one oligonucleotide hybridised to the target sequence is achieved by the nearby hybridisation of a second oligonucleotide labelled with an energy donor fluorophore or dye. In such FRET probe configurations there is no need for any complementarity to exist between the two labelled oligonucleotides

A tribrid-like design has been proposed in a special application of HyBeacon® probes directed towards discriminating the number of repeating sequence elements in Short Tandem Repeats (STRs) In this STR example, the HyBeacon® probe and a ‘blocker’ oligonucleotide have a junction to anchor probe hybridisation against the target sequence (WO 09/053679, FIG. 12 and claim 34). The fluorophores in this example are conventionally placed within the probe sequence so as to hybridise with target specific sequence and are not on the junction between probe and blocker oligonucleotides

The present invention therefore provides a novel molecular probing complex that is explained in more detail in the following description and examples.

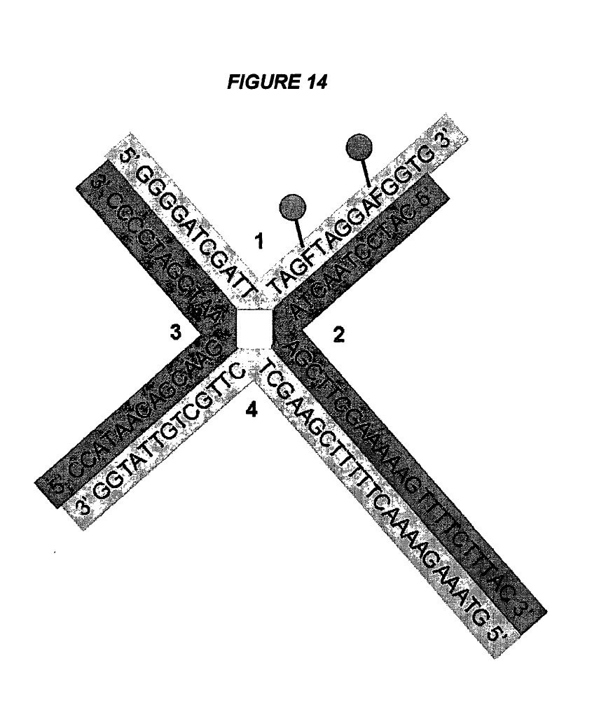

There is also proposed an alternate method for universal sequence analysis in the examples which use an oligonucleotide probe which does not hybridise directly with target sequences. This method uses two universal complement oligonucleotides to hybridise to adjacent sections of target sequence which, once bound, create the target site for probe hybridisation. Both universal complement oligonucleotides comprise target specific target sequences and universal element sequences required for probe hybridisation. The combination of probe, two universal complement oligonucleotides and target forms a four-strand (cruciform) structure which allows sequence detection and discrimination (see FIG. 14). This method will enable a single HyBeacon® probe to detect many unrelated target sequences, requiring synthesis of only two inexpensive unlabelled UC oligonucleotides. The demand for the 2-phase synthesis method is reduced with this cruciform method, but cost reduction can still be achieved by adding target specific sequence onto the universal elements of UC oligonucleotides. Furthermore, since the probes of this example possess two universal element sequences, the 2-phase synthesis method can be used to add different unlabelled universal element sequences onto labelled universal element components.

A similar four-strand structure has been described by Kolpashchikov (US 2009/0176318) which uses “binary” probes for target detection, requiring co-operative oligonucleotide binding to allow hybridisation of molecular beacon probes. Two unlabelled oligonucleotides are used to bind to adjacent sections of target sequence. These oligonucleotides require a linker between the target specific sequences and molecular beacon binding regions. These linkers can be any molecule (including unhybridised nucleotides) that is flexible enough to let the molecular beacon form a double helix when the target specific sequences of unlabelled oligonucleotides are hybridised with target. The molecular beacon does not hybridise with unlabelled oligonucleotides in the absence of target sequence. This binary probe method is restricted to:
(i) Molecular beacon probes comprising stem-loop secondary structures with 5′ and 3′ fluorophore and quencher labels or;
(ii) Binary probes that include an aptamer sequence to bind a dye such as malachite green, where fluorescence emission increases dramatically upon hybridisation to target sequence.
(iii) Unlabelled oligonucleotides with analyte binding and molecular beacon binding sequences separated by a flexible linker.

US 2009/0176318 describes a method for SNP analysis that uses an oligonucleotide labelled with a quencher, an oligonucleotide labelled with a fluorophore and a five (or 4-7) nucleotide gap between the two which contains the position of the SNP. Short oligonucleotides then hybridise in the gap between fluorophore and quencher oligonucleotides to facilitate a fluorescence increase only if the short oligonucleotide matches the SNP sequence present.

The universal probes described herein in the four-strand embodiment are not restricted to molecular beacons and do not require a flexible linker between target specific sequence and universal elements. Analysis of SNPs is achieved with a single fluorescent oligonucleotide and dye labels are attached to internal nucleotides.

In contrast the probes of this current application are not designed to detect target sequences below 30° C., preferably detecting fully complementary target sequences in the temperature range of 40° C. to 65° C., preferably between 41° C. and 65° C., and more preferably between 45° C. and 65° C. Furthermore, the probes of the invention are able to detect variant sequences which exhibit nucleotide mismatches in target polynucleotides.

In a first aspect of the invention there is provided, a method of detecting the presence of a target polynucleotide and/or sequence variations within the target polynucleotide in a sample of interest, comprising the steps of:
(i) providing a probe comprising two independent partner oligonucleotide components comprising:
   a) a first oligonucleotide having a first and second section, wherein the first section comprises a nucleotide sequence that is labelled with at least one visually detectable label and is not capable of hybridising to the nucleotide sequence of the target polynucleotide; and wherein the second section comprises a nucleotide sequence that is capable of hybridising to a portion of the sequence of the target polynucleotide said second section of the first oligonucleotide having a melting temperature (Tm) of between 25 and 50° C.; and
   b) a second oligonucleotide having a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to said second section of the second oligonucleotide having a melting temperature (Tm) of between 40 and 70° C.;
(ii) exposing the sample of interest to the probe of (i), such that in the presence of the target polynucleotide, a triple hybridisation event can occur which involves (a) the hybridisation of the first section of the first oligonucleotide to the first section of the second oligonucleotide; and (b) the hybridisation of both the second section of the first oligonucleotide and the second section of the second oligonucleotide to the target polynucleotide;
(iii) detecting a change in the visually detectable label, wherein a change in the visually detectable label indicates the presence of the target polynucleotide and/or the sequence variations within the target polynucleotide; and
(iv) analysing the change in the visually detectable label using either melting curve analysis or annealing curve analysis.

In one embodiment the first oligonucleotide is labelled internally with at least one visually detectable label.

The length of the oligonucleotides of the invention are preferably such that it is suitable for hybridising with the target polynucleotide, to provide a stable hybrid whose melting temperature depends on the exact sequence of the target. Oligonucleotides containing less than 15 nucleotide residues in many cases do not form sufficiently stable hybrids in temperatures ranging between 35° C. and 70° C., particularly where the two hybridising sequences are not fully complementary, although they can be used in some circumstances. Linear oligonucleotides, which are longer than about 30 nucleotide residues may form hybrids whose melting temperature is relatively insensitive to the possible presence of a single nucleotide mismatch, although they can be used in some circumstances. The junctions formed with 3-strand (tribrid) and 4-strand (cruciform) structures are destabilising and the oligonucleotides of the invention are typically longer than standard linear probes that bind directly to target sequences without requiring interaction with additional oligonucleotides. Formation of 3-strand (tribrid) or 4-strand (cruciform) structures are required to provide probes with sufficient stability for target detection. Oligonucleotide design guidelines are discussed below and in further detail in example 20.

Typically for tribrid analysis, the junction probe oligonucleotides are from 15 to 40 nucleotide residues in length, preferably from 20 to 30 nucleotide residues in length. The design of fluorescently labelled probes ensures that oligonucleotides do not form stable interactions with target sequence in the absence of adjacent universal complement hybridisation. The unlabelled universal complement oligonucleotides are typically longer and of higher Tm compared with junction probe oligonucleotides, especially when sequence variations such as SNPs are located within the target sequence of the junction probe. Typically, the universal complement oligonucleotides are from 20-50 nucleotide residues in length, preferably from 25 to 40 nucleotide residues in length.

Typically, the oligonucleotides of the probe system have a sequence complementary to a target polynucleotide sequence. Thus, the oligonucleotide is capable of hybridising to the target polynucleotide sequence under appropriate conditions. Thus, unless the context indicates otherwise, by “complementary” we include the meaning that the oligonucleotide is able to hybridise to a target polynucleotide sequence. The oligonucleotide may be fully complementary to the target polynucleotide sequence (i.e. there is a perfect match in terms of base pairing between the oligonucleotide), or the oligonucleotide may be partially complementary to the target polynucleotide sequence (i.e. there are one or more mismatches between the oligonucleotide and the target polynucleotide sequence, but the oligonucleotide is still able to hybridise). Typically, when the oligonucleotide is partially complementary with the target polynucleotide, there are fewer than 5 mismatches, preferably 1 or 2 or 3 or 4 mismatches, more preferably one mismatch. Conveniently, the target specific sections of the first and second oligonucleotides have at least 70% sequence identity to their complementary targets, more preferably at least 80% or at least 85% or at least 90% or at least 95%. For example, for an oligonucleotide of 20 residues, there may be 6 or 4 or 3 or 2 or 1 mismatch with the target.

Where reference is made to “hybridisation” or the ability of an oligonucleotide and/or primer to “hybridise” to another nucleotide sequence, the skilled person will understand that such hybridisation is capable of occurring under conditions used for melting or annealing curve analysis, typically performed between 15° C. and 95° C., preferably between 20° C. and 70° C.

The melting temperature of 3-strand (tribrid) structures are typically between 30° C. and 70° C. The preferred melting temperatures of universal probe structures with fully complementary target sequences is between 40° C. to 65° C., preferably between 41° C. and 65° C., and more preferably between 45° C. and 65° C. for target detection and differentiation of sequence variants. The hybridisation conditions that are most appropriate for a particular probe-target reaction will be readily known to the skilled person, but will typically be the hybridisation conditions for any standard amplification buffer used in, e.g. PCR or another isothermal amplification.

The probe system of the first aspect therefore comprises two separate oligonucleotides, the first oligonucleotide is also referred to herein as the “universal probe” or “junction probe” and the second oligonucleotide is also referred to herein as the “universal complement”.

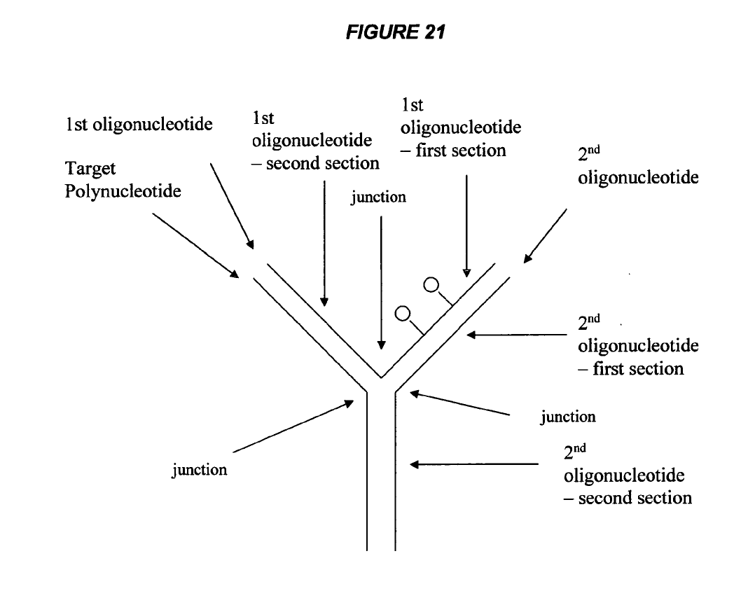

FIG. 21 provides a simple schematic representation of the first and second oligonucleotides and their interaction with the target polynucleotide and each other.

The target polynucleotide may be any polynucleotide or sequence variation therein of interest. The target polynucleotide may therefore be derived from any source, depending on the application for which the detection is being performed, where such sources include organisms that comprise nucleic acids, i.e. viruses; prokaryotes, e.g. bacteria, archaea and cyanobacteria; and eukaryotes, e.g. members of the kingdom protista, such as flagellates, amoebas and their relatives, amoeboid parasites, ciliates and the like; members of the kingdom fungi, such as slime molds, acellular slime molds, cellular slime molds, water molds, true molds, conjugating fungi, sac fungi, club fungi, imperfect fungi and the like; plants, such as algae, mosses, liverworts, hornworts, club mosses, horsetails, ferns, gymnosperms and flowering plants, both monocots and dicots; and animals, including sponges, members of the phylum cnidaria, e.g. jelly fish, corals and the like, combjellies, worms, rotifers, roundworms, annelids, molluscs, arthropods, echinoderms, acorn worms, and vertebrates, including reptiles, fishes, birds, snakes, and mammals, e.g. rodents, primates, including humans. In some embodiments, the target polynucleotide may be from a synthetic source.

The “Sample of interest” may be any sample derived from any source and includes both in vitro and in vivo samples. The sample may be directly derived from a living organism, such as tissue, cell or blood sample or may alternatively be an environmental sample that may or may not comprise at least one organism (either dead or alive) for example a microorganism.

The target polynucleotide and/or the sequence variations within the target polynucleotide may only be detectable after the triple hybridisation event has occurred.

As used herein, “nucleic acid” means either DNA or RNA, single-stranded or double-stranded, and any chemical modifications thereof. Modifications include, but are not limited to, those which provide other chemical groups that incorporate additional charge, polarisability, hydrogen bonding, electrostatic interaction, and functionality to the nucleic acid. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

The visually detectable label generates a detectable signal and this may be detected after a triple hybridisation event has occurred by measuring the signal at any point during or after the probe reaction. Measurement of the signal may be qualitative or quantitative and is conducted using equipment appropriate for the particular visually detectable label being used.

The second section of the first oligonucleotide and the second section of the second oligonucleotide may hybridise to either contiguous or non-contiguous sequences of the target polynucleotide. If the hybridising sections of the target are contiguous there are no unhybridised nucleotides between them. If the hybridising sections of the target are non-contiguous optionally there may be 1, 2, 3, 4, 5, 6, 7, 8, or 9 unhybridised nucleotides between them.

In one embodiment the melting temperature (Tₘ) of the target polynucleotide of the second section of the second oligonucleotide is greater than the Tₘ of the target polynucleotide of the second section of the first oligonucleotide to ensure identification of mismatched sequences. Alternatively, the Tₘ of the target polynucleotide of the second section of the second oligonucleotide is the same or substantially the same (or substantially similar) as the Tₘ of the target polynucleotide of the second section of the first oligonucleotide.

The Tₘ of the second section of the first oligonucleotide is typically between 25° C. and 50° C. and is preferably between 30° C. and 40° C. The Tₘ of the second section of the second oligonucleotide is typically between 40° C. and 70° C. and is preferably between 50° C. and 60° C. When sequence polymorphisms are located within the target sequence of the first (junction probe) oligonucleotide, the Tm of the second section of the second (UC) oligonucleotide is preferably 10° C. higher than the second section of the first oligonucleotide. Polymorphisms located within the target sequence of the second (unlabelled UC) oligonucleotide benefit from designs where the second section of the second oligonucleotide is substantially similar to the Tₘ of the target polynucleotide of the second section of the first oligonucleotide, such that destabilisation of the second oligonucleotide determines the overall stability of the 3-strand tribrid structure.

The Tm of an oligonucleotide is the temperature in ° C. at which 50% of the molecules in a population of a single-stranded oligonucleotide are hybridised to their complementary sequence and 50% of the molecules in the population are not-hybridised to said complementary sequence. The Tm may be determined empirically, for example Tm may be measured using melting or annealing curve analysis, e.g. using a Roche LightCycler 480 instrument on a 96-well white plate. The Tm may be tested in standard reaction buffer for the apparatus used. Melting peaks may be generated from melt curve data by the LightCycler 480 analysis function (−dF/dT). Tms are calculated by using a manual Tm option to identify the lowest point in the inverse melt peak. The software for the apparatus is also capable of automatically detecting inverted peaks and measure Tms. The melting temperature is fundamentally determined by the temperature of a solution containing the oligonucleotides being slowly raised, while continuously observing a fluorescence signal, in order to construct a graph of the negative derivative of fluorescence signal intensity with respect to temperature (−dF/dT) against temperature. The melting temperature (Tm) of the hybrid appears as a peak, and provides information about the sequence of the polynucleotide target. The Tms generated through melting analysis of the oligonucleotide of the invention may be used to distinguish polymorphic targets.

Where reference is made to a Tm for hybridisation involving part of an oligonucleotide, the relevant Tm is considered to be the Tm that can be calculated from a nearest neighbour analysis of the sequence involved.

As an alternative, the analysis may be performed by cooling the solution slowly from high temperature and determining the annealing temperature (Tₐ). The annealing temperature is typically between 30° C. and 70° C. The preferred annealing temperatures of universal probe structures with fully complementary target sequences is between 40° C. to 65° C., preferably between 41° C. and 65° C., and more preferably between 45° C. and 65° C.

In a second aspect of the invention there is provided a method of detecting the presence of a target polynucleotide and/or sequence variations within the target polynucleotide in a sample of interest, comprising the steps of:
(i) providing a probe system comprising three independent partner oligonucleotide components comprising:
   a) a first oligonucleotide having a first and second section, wherein the first oligonucleotide is not capable of hybridising to the sequence of the target polynucleotide and wherein the first section comprises a nucleotide sequence that is labelled with at least one visually detectable label that is capable of hybridising to a portion of the sequence of a second oligonucleotide; and wherein the second section comprises a nucleotide sequence that is capable of hybridising to a portion of the sequence of a third oligonucleotide, said second section of the first oligonucleotide having a melting temperature (Tm) of between 40 and 70° C.;
   b) a second oligonucleotide having a first and second section, wherein the first section comprises a nucleotide sequence which is capable of hybridising to a nucleotide sequence of the first section of the first oligonucleotide; and the second section comprises a nucleotide sequence which is capable of hybridising to a nucleotide sequence of the target polynucleotide, said second section of the second oligonucleotide having a melting temperature (Tm) of between 25 and 50° C.; and
   c) a third oligonucleotide having a first and second section, wherein the first section comprises a nucleotide sequence which is capable of hybridising to a nucleotide sequence of the second section of the first oligonucleotide; and the second section comprises a nucleotide sequence which is capable of hybridising to a nucleotide sequence of the target polynucleotide that is adjacent to the nucleotide sequence that the second section of the second oligonucleotide is complementary to, said second section of the third oligonucleotide having a melting temperature (Tm) of between 40 and 70° C.;
(ii) exposing the sample of interest to the probe system of (i), such that in the presence of the target polynucleotide, a quadruple hybridisation event can occur which involves (a) the hybridisation of the first section of the first oligonucleotide to the first section of the second oligonucleotide; and (b) the hybridisation of the second section of the first oligonucleotide to the first section of the third oligonucleotide; and (c) the hybridisation of both the second section of the second oligonucleotide and the second section of the third oligonucleotide to the target polynucleotide;
(iii) detecting a change in the visually detectable label, wherein a change in the visually detectable label indicates the presence of the target polynucleotide and/or the sequence variations within the target polynucleotide; and
(iv) analysing the change in the visually detectable label using either melting curve analysis or annealing curve analysis.

In one embodiment the first oligonucleotide is labelled internally with at least one visually detectable label.

The cruciform (3 oligonucleotides plus target) analysis utilises two unlabelled universal complement oligonucleotides, the second oligonucleotide being a lower stability “probing UC” to hybridise to the target sequence in the region of polymorphism and the third oligonucleotide being a higher stability “non-probing UC” which binds to non-polymorphic target sequence. The second oligonucleotides are from 15 to 40 nucleotide residues in length, preferably from 20 to 30 nucleotide residues in length. The third oligonucleotides are typically longer and of higher Tm comprising 20-50 nucleotide residues, preferably from 25 to 40 nucleotide residues in length. The first oligonucleotide does not hybridise directly with the target polynucleotide and as such probe melting temperature is determined by dissociation of the second oligonucleotide. The first oligonucleotides are between 18 and 35 nucleotide residues in length, preferably between 20 and 30 nucleotides in length.

The melting temperature of 4-strand (cruciform) structures are typically between 30° C. and 70° C. The preferred melting temperatures of universal probe structures with fully complementary target sequences is between 40° C. to 65° C., preferably between 41° C. and 65° C., and more preferably between 45° C. and 65° C. or between 40° C. and 60° C. for target detection and differentiation of sequence variants.

The Tₘ of the second section of the second oligonucleotide is typically between 25° C. and 50° C. and is preferably between 30° C. and 40° C. The Tₘ of the second section of the third oligonucleotide is typically between 40° C. and 70° C. and is preferably between 50° C. and 60° C. When sequence polymorphisms are located within the target sequence of the second oligonucleotide, the Tm of the second section of the third oligonucleotide is preferably 10° C. higher than the second section of the second oligonucleotide. The Tm of the first oligonucleotide is typically between 40° C. and 70° C. and is preferably between 50° C. and 60° C. The Tm of the first oligonucleotide is preferably 10° C. higher than the second section of the first oligonucleotide.

In this second aspect, the target polynucleotide and/or the sequence variations within the target polynucleotide may only be detectable after the quadruple hybridisation event has occurred. Furthermore, the second section of the first oligonucleotide and the second section of the third oligonucleotide may hybridise to contiguous or non-contiguous sequences of the target polynucleotide. If the hybridising sections of the target are contiguous there are no unhybridised nucleotides between them. If the hybridising sections of the target are non-contiguous there may be 1, 2, 3, 4, or 5 unhybridised nucleotides between them.

Similarly to between the second sections of the first and second oligonucleotides of the first aspect of the invention, the Tₘ of the target polynucleotide of the second section of the third oligonucleotide may be greater than or substantially the same (or substantially similar) to the Tₘ of the target polynucleotide of the second section of the first oligonucleotide to ensure identification of mismatched sequences.

The following features apply to both the first and second aspects of the invention.

In one embodiment, the first and second sections of the first oligonucleotide are joined (by joined we also mean linked or attached) at a junction, said junction being positioned at least two nucleotides from the visually detectable label positioned 5′-most when the first oligonucleotide has been synthesised in the 3′ to 5′ direction. This is equivalent to the 5′-most visually detectable label being positioned at least two nucleotides from the junction. By 5′-most, we mean that within or on the first oligonucleotide, the visually detectable label is positioned/located most 5′, i.e. nearest or closest to the 5′ end of the oligonucleotide. Said junction may alternatively be positioned at least two nucleotides from the visually detectable label positioned 3′-most when the first oligonucleotide has been synthesised in the 5′ to 3′ direction

In alternative embodiments, the distance between the junction and the visually detectable label is at least three, or at least four nucleotides. In a further embodiment, the distance between the junction and the visually detectable label is between two and four, i.e. two, three or four, nucleotides from the junction.

In other words, a preferred minimum distance between the junction and the closest visually detectable label is at least two, at least three, at least four, or between two and four nucleotides. This positioning/distance (both in terms of number of nucleotides and 5′most) is along the oligonucleotide sequence itself, and does not represent 3D structure or spacing of an equivalent distance to that number of nucleotides.

It is to be noted that the minimum distance between the junction and the closest visually detectable label specified above is reflective of a desire to generate positive melting peaks to differentiate between target sequences. If, however, negative (inverted) melting peaks are desired for target differentiation and/or are useful in a specific context, then the distance between the junction and the closest visually detectable label will be different, e.g. 1 nucleotide. Depending on the effect that, for example, probe sequence may have on the magnitude of fluorescence or quenching, then as the number of nucleotides increases between the junction and the closest visually detectable label then the melting curve analysis generates different results. For example, 1 nucleotide generating negative peaks and generating positive peaks, so the general effect will be a switch from negative to positive melting peaks that serve to differentiate between target sequences.

Accordingly, the first and second sections of the first oligonucleotide are joined at a junction, said junction being positioned one nucleotide from the visually detectable label positioned 5′-most when the first oligonucleotide has been synthesised in the 3′ to 5′ direction; or said junction being positioned at one nucleotide from the visually detectable label positioned 3′-most when the first oligonucleotide has been synthesised in the 5′ to 3′ direction.

The junction between the first and second sections of the oligonucleotide is where the 5′ end of the first section and the 3′ end of the second section join (also described as where the two sections are linked or attached to one another).

The positioning of internally visually detectable labelled nucleotides is designed to enhance fluorescence. However, it is to be noted that the sequence context of the probe may also influence the magnitude of fluorescence change upon probe-target hybridisation. Such as exemplified in WO 2007/010268 A2—see Example 6 (Probe signal-to-noise) and Table 2, pages 47-54. Furthermore, the effect of spacing between fluorophores is shown in WO 2007/010268 A2—see Example 11 and Table 7, pages 67-69. It would be within the technical capability of one of skill in the art to readily determine, without undue experimentation, the most appropriate probe sequence context to combine with the most appropriate placement of visually detectable labels (both in terms of distance of labels from junction and inter-label spacing), in order to produce the desired level (magnitude) of fluorescence. Indeed, depending on the sequence context and, especially, the distance between the junction to 5′-most visually detectable label (number of nucleotides), the desired sequence differentiation effect could actually be a quenching of fluorescence rather than enhancement of fluorescence. Universal elements such as in the probes of the current invention have been shown to yield positive melting peaks in a reproducible manner even when applied to multiple different target sequences (see example 11). Hence, the universal system overcomes unknown effects related to target sequence on fluorescence.

It is to be further noted that whilst the probe junction would be located 5′ of the most 5′-most visually detectable label (for probes synthesised in the 3′ to 5′ direction), this would nevertheless be reversed if probes were synthesised in the 5′ to 3′ direction. Whilst such is less likely to be carried out, it is nevertheless technically possible. Accordingly, the above-recited passages must be read as applying mutatis mutandis to probes synthesised in the 5′ to 3′ direction also.

For the avoidance of doubt, 3′ to 5′ synthesised probes have the first section of the oligonucleotide located 3′ of the second section; whereas 5′ to 3′ synthesised probes have the second section of the oligonucleotide located 3′ of the first section.

The CDK-JP probe exemplified in Example 2 herein includes the second section of the oligonucleotide located 3′ of the first section of the oligonucleotide and models a probe produced by 5′ to 3′ syntheses methods.

In one embodiment, the independent partner oligonucleotides are not labelled with a quencher. By quencher, we mean a separate molecule attached to the oligonucleotide that acts to quench the activity of a visually detectable label. A quencher in this context does not include the oligonucleotides of the invention or any portion therein. Accordingly, the invention does not require the presence of a molecule (i.e. a quencher) to prevent detection of the visually detectable label prior to the oligonucleotides binding to the target as part of the multiple oligonucleotide structures of the invention (the tribrid or cruciform structures discussed herein). Therefore, in this particular embodiment, fluorophore-quencher pairs are not attached to the oligonucleotides of the invention.

In one embodiment, the hybridisation of the first section of the first oligonucleotide to the first section of the second oligonucleotide improves the detection of the visually detectable label. In an alternative embodiment the hybridisation of the first section of the first oligonucleotide to the first section of the second oligonucleotide quenches the visually detectable label. The choice of a visually detectable label and the oligonucleotide sequences will determine whether hybridisation enhances or quenches fluorescence.

In some embodiments, the first section of the second oligonucleotide is also labelled with a visually detectable label, and in respect of the second aspect of the invention, the first section of the third oligonucleotide may also be labelled with a visually detectable label.

The visually detectable label is preferably a fluorophore or a dye. The fluorophores for the labelled oligonucleotide pairs may be selected so as to be from a similar chemical family or a different one, such as cyanine dyes, xanthenes or the like. Fluorophores of interest include, but are not limited to fluorescein dyes (e.g. fluorescein dT, 5-carboxyfluorescein (5-FAM), 6-carboxyfluorescein (6-FAM), 2′, 4′, 1,4,-tetrachlorofluorescein (TET), 2′, 4′, 5′, 7′, 1,4-hexachlorofluorescein (HEX), and 2′,7′-dimethoxy-4′,5′-dichloro-6-carboxyfluorescein (JOE)), cyanine dyes such as Cy5, dansyl derivatives, rhodamine dyes (e.g. tetramethyl-6-carboxyrhodamine (TAMRA), and tetrapropano-6-carboxyrhodamine (ROX)), DABSYL, DABCYL, cyanine, such as Cy3, anthraquinone, nitrothiazole, and nitroimidazole compounds, or other non-intercalating dyes. Fluorophores of interest are further described in International Patent Applications WO 01/42505 and WO 01/86001.

As used herein, “fluorophore” (also referred to as fluorescent group) refers to a molecule that, when excited with light having a selected wavelength, emits light of a different wavelength.

Examples of fluorophore containing oligonucleotides include the HyBeacon® II probes described in WO 2007/010268.

Preferably, the first section of the first oligonucleotide is labelled internally with two or more visually detectable labels, optionally three or more or four or more. The maximum number of visually detectable labels that can be present is determined by the length of the nucleotide sequence is calculated as a round down of the sequence length divided by three (e.g. a length of 20 nucleotides can comprise up to 6 labels). The labels on the first section of the first oligonucleotide may each be different labels, i.e. the first oligonucleotide may carry multiple different labels.

Fluorescently labelled oligonucleotides may contain modified bases such as phosphorothioate-modified bases. The number of phosphodiester linkages replaced by phosphorothioates in any given oligonucleotide/primer can range from none to all of the phosphodiester bonds being replaced by phosphothioates, for example one, two, three, four or more. The oligonucleotide(s) preferably contain phosphorothioates at least one, at least or at least three of the internal bases of the oligonucleotide. In one embodiment the phosphorothioate-modified bases (where there is more than one) are separated by at least one, e.g. one to three, unmodified (phosphorodiester) bases, for example alternate bases within the oligonucleotide(s)/primer(s) may be phosphorothioates. See, for example, PCT/GB2012/050645, for discussion of phosphorothioate incorporation patterns that are considered also to be useful in relation to the present invention.

The improvement in detection may be achieved by either an enhancement of fluorescent emissions or by a quenching of fluorescent emissions.

The magnitude of fluorescence change by enhancement or quenching upon hybridisation is at least 10% higher or lower than the fluorescence observed with single-stranded probe, respectively. Preferably the percentage change is greater than 20%. More preferably the percentage change is greater than 50%.

Fluorescence enhancement can occur upon target hybridisation when fluorophore-labelled residues are placed in all sequence environments. Placing fluorophore-labelled residues adjacent to G's may result in the highest levels of fluorescence enhancement in the duplex state. However, fluorescence enhancement upon target hybridisation will also occur when fluorophore-labelled residues are located within regions of high C abundance.

All DNA bases are able to quench fluorescence to some extent, where G has the greatest such ability. For the avoidance of doubt, the term “associated quencher” does not include DNA bases which form part of the oligonucleotide. Fluorophores on the oligonucleotides of the invention may interact with the bases of single-stranded DNA such that fluorescence is quenched. Gs may modulate fluorescence strength but all dequench significantly on hybridisation. Fluorescence from the internally attached fluorophores of the oligonucleotides of the invention is always enhanced upon duplex formation irrespective of the location and abundance of guanines in the probe and target strand. Dye-Dye interactions also cause fluorescence quenching in the single-stranded state. Typically probe hybridisation removes these dye-dye interactions causing fluorescence enhancement permitting target detection.

The labelled oligonucleotides of the invention emit significantly greater amounts of fluorescence when hybridised to complementary nucleic acid sequences compared with the single-stranded (non-hybridised) conformation despite the absence of a quencher component.

In one embodiment, the methods of the first and second aspects of the invention further comprise the optional step of (iv) analysis of the change in the visually detectable label.

Such analysis may, for example, be undertaken using either melting curve analysis or annealing curve analysis. The analysis may also be undertaken using any other measurement or calculation which enables the identification of a change in a visually detectable label, such change reflecting the hybridisation caused by the presence of a particular sequence and/or polymorphism within the target polynucleotide. Examples of such methods include those disclosed in Halpern & Ballantyne (2011) J. Forensic Sci, 56 pp 36-45. Positive melting curves are associated with enhancement of fluorescence, whereas negative melting curves are associated with quenching of fluorescence.

In some embodiments of the invention, the first section of the first oligonucleotide is longer than the first section of the second oligonucleotide resulting in an overhanging nucleotide sequence. In an alternative embodiment of the invention, the first section of the second oligonucleotide is longer than the first section of the first oligonucleotide resulting in an overhanging nucleotide sequence. Sequence overhangs may be 1-10 nucleotides, for example 2-5 nucleotides.

Alternatively, the first section of the first oligonucleotide has the same nucleotide sequence length as the first section of the second oligonucleotide, preferably wherein the Tₘ is optimised to prevent the first section of the first oligonucleotide hybridising to the first section of the second oligonucleotide in the absence of target polynucleotide.

Optimisation of the lengths and Tₘs of the first sections of first and second oligonucleotides is first performed by nearest-neighbour analysis to design oligonucleotide sections with Tₘs between 5° C. and 30° C. Modification of the lengths of the first sections of first and second oligonucleotides is then performed experimentally to ensure target detection whilst preventing the generation of melting peaks involving first and second oligonucleotides in the absence of target sequence.

In one embodiment, the methods may be used to detect the presence of specific known polymorphisms. For example, known polymorphisms that are indicative of the presence of a particular phenotype, disease state, disease susceptibility, predicted response to medication or specific strains of micro-organisms. The known polymorphisms may be detected conducting a melting or annealing analysis which generates defined melting (Tₘ) or annealing (Tₐ) peaks that may be cross referenced to identify the presence of the known polymorphisms. The methods may therefore be used in various fields including but not limited to diagnostics, forensic science, paternity and relationship testing, linkage mapping, microbial typing or traceability within the food chain.

The methods of the invention are not limited to detecting known polymorphisms and may be used to detect an unknown polymorphism. This may be achieved by generating a previously unknown melting peak Tₘ or annealing peak Tₐ.

In some embodiments of the invention interaction of the first and second oligonucleotides is prevented in the absence of target sequence. However, a direct interaction of first and second oligonucleotides can be a useful feature in other embodiments to generate melting/annealing peaks at lower temperatures than target peaks. It is possible to utilise the method of the invention to confirm the absence of the target polynucleotide in a sample, due to the presence of only a melting peak specific to the hybridisation of the first and second oligonucleotides and/or the first and third oligonucleotides.

The methods of the invention may be used in target detection, SNP genotyping, or detection of length polymorphisms and repetitive sequences. (French et al. 2001, French et al. 2002, French et al. 2007, French et al. 2008).

Polynucleotide targets which may be identified using the methods of the invention include any nucleic acid-containing targets, such as native DNA or RNA. The nucleic acids may where appropriate include sequences that include any of the known base analogs of DNA and RNA such as 4 acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine and 2,6-diaminopurine; or they may contain PNAs.

The oligonucleotides used in the method of the invention may include such base analogs or PNAs as appropriate, though this may not be typical.

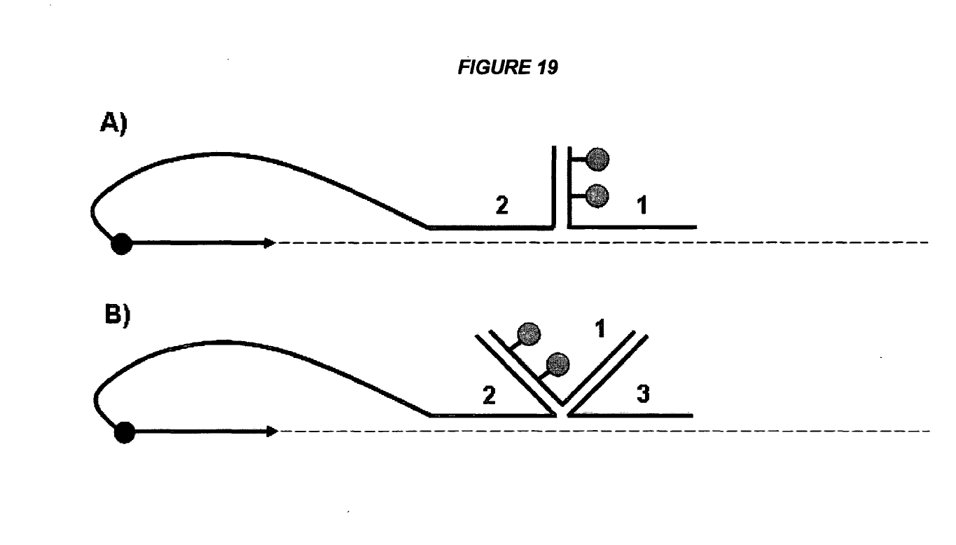

The method of the invention may form part a Polymerase Chain Reaction (PCR) wherein a section of the probe constitutes a PCR primer (see FIG. 19 which shows how this would work for both the first and second aspects of the invention).

The method may further comprise the use of a 3′ blocking modification, e.g. a 3′ blocking moiety, to prevent unwanted amplification and dimer formation during PCR. The use of 3′ blocking in PCR reactions is described in (French et al. 2001, Ben Gaied et al. 2010). Examples of blocking moieties that may be used are pyrene dT, phosphate and propanol.

The method may alternatively utilise no 3′ blocking because the 3′ end of the section of the probe constituting the PCR primer is located within the first section of the first oligonucleotide.

The method of the invention may also be used in conjunction with an ‘isothermal’ methodology of nucleic amplification such as a Loop-mediated isothermal amplification (LAMP) method, wherein a section of the probe constitutes a LAMP primer. (see Notomi et al. (2000)).

In a third aspect of the invention there is provided a probe system as defined and used in either the first or the second aspects of the invention.

In a fourth aspect of the invention, there is an oligonucleotide hybridisation structure comprising three oligonucleotides;
(a) a target polynucleotide;
(b) a first oligonucleotide having a first and second section, wherein the first section comprises a nucleotide sequence that is labelled with at least one visually detectable label and is not capable of hybridising to the nucleotide sequence of the target polynucleotide; and wherein the second section comprises a nucleotide sequence that is capable of hybridising to a portion of the sequence of the target polynucleotide, said second section of the first oligonucleotide having a melting temperature (Tm) of between 25 and 50° C.; and
(c) a second oligonucleotide having a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to, said second section of the second oligonucleotide having a melting temperature (Tm) of between 40 and 70° C.;
   wherein the first section of the first oligonucleotide is hybridised to the first section of the second oligonucleotide; and both the second section of the first oligonucleotide and the second section of the second oligonucleotide are hybridised to the target polynucleotide.

In one embodiment the first oligonucleotide is labelled internally with at least one visually detectable label.

In a fifth aspect of the invention there is provided an oligonucleotide hybridisation structure comprising four oligonucleotides;
(a) a target polynucleotide;
(b) a first oligonucleotide having a first and second section, wherein the first oligonucleotide is not capable of hybridising to the sequence of the target polynucleotide and wherein the first section comprises a nucleotide sequence that is labelled with at least one visually detectable label that is capable of hybridising to a portion of the sequence of a second oligonucleotide; and wherein the second section comprises a nucleotide sequence that is capable of hybridising to a portion of the sequence of a third oligonucleotide, said second section of the first oligonucleotide having a melting temperature (Tm) of between 40 and 70° C.;
(c) a second oligonucleotide having a first and second section, wherein the first section comprises a nucleotide sequence which is capable of hybridising to a nucleotide sequence of the first section of the first oligonucleotide; and the second section comprises a nucleotide sequence which is capable of hybridising to a nucleotide sequence of the target polynucleotide, said second section of the second oligonucleotide having a melting temperature (Tm) of between 25 and 50° C.; and
(d) a third oligonucleotide having a first and second section, wherein the first section comprises a nucleotide sequence which is capable of hybridising to a nucleotide sequence of the second section of the first oligonucleotide; and the second section comprises a nucleotide sequence which is capable of hybridising to a nucleotide sequence of the target polynucleotide that is adjacent to the nucleotide sequence that the second section of the second oligonucleotide is complementary to, said second section of the third oligonucleotide having a melting temperature (Tm) of between 40 and 70° C.;
   wherein the first section of the first oligonucleotide is hybridised to the first section of the second oligonucleotide; the second section of the first oligonucleotide is hybridised to the first section of the third oligonucleotide; and both the second section of the second oligonucleotide and the second section of the third oligonucleotide are hybridised to the target polynucleotide.

In one embodiment the first oligonucleotide is labelled internally with at least one visually detectable label.

The following features relate to both the fourth and fifth aspects of the invention. The oligonucleotide hybridisation structure may comprise a first section of the first oligonucleotide that is longer than the first section of the second oligonucleotide resulting in an overhanging nucleotide sequence.

The second section of the first oligonucleotide and the second section of the third oligonucleotide may hybridise to either a contiguous sequences or a non-contiguous sequences of the target polynucleotide.

The first section of the second oligonucleotide and/or the first section of the third oligonucleotide may also be labelled with a visually detectable label. Such a visually detectable label may be a fluorophore or a dye, for example, as defined above.

The first section of the first oligonucleotide may be labelled internally with two or more visually detectable labels, optionally three or more, and such labels may each be different.

In a sixth aspect of the invention, there is provided a use of the probe system of the third aspect in a method of detecting a target, SNP genotyping, or detecting length polymorphisms and repetitive sequences. These uses may be applicable in various fields including but not limited to diagnostics, forensic science, paternity and relationship testing, linkage mapping, microbial typing or traceability within the food chain.

In a seventh aspect of the invention there is provided a kit of parts comprising:
(a) a first oligonucleotide having a first and second section, wherein the first section comprises a nucleotide sequence that is labelled with at least one visually detectable label and is not capable of hybridising to the nucleotide sequence of the target polynucleotide; and wherein the second section comprises a nucleotide sequence that is capable of hybridising to a portion of the sequence of the target polynucleotide, said second section of the first oligonucleotide having a melting temperature (Tm) of between 25 and 50° C.; and
(b) a second oligonucleotide having a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to, said second section of the second oligonucleotide having a melting temperature (Tm) of between 40 and 70° C.; and
(c) instructions for use.

In one embodiment the first oligonucleotide is labelled internally with at least one visually detectable label.

The kit may alternatively comprise:
(a) a first oligonucleotide having a first and second section, wherein the first oligonucleotide is not capable of hybridising to the sequence of the target polynucleotide and wherein the first section comprises a nucleotide sequence that is labelled with at least one visually detectable label that is capable of hybridising to a portion of the sequence of a second oligonucleotide; and wherein the second section comprises a nucleotide sequence that is capable of hybridising to a portion of the sequence of a third oligonucleotide, said second section of the first oligonucleotide having a melting temperature (Tm) of between 40 and 70° C.;
(b) a second oligonucleotide having a first and second section, wherein the first section comprises a nucleotide sequence which is capable of hybridising to a nucleotide sequence of the first section of the first oligonucleotide; and the second section comprises a nucleotide sequence which is capable of hybridising to a nucleotide sequence of the target polynucleotide, said second section of the second oligonucleotide having a melting temperature (Tm) of between 25 and 50° C.;
(c) a third oligonucleotide having a first and second section, wherein the first section comprises a nucleotide sequence which is capable of hybridising to a nucleotide sequence of the second section of the first oligonucleotide; and the second section comprises a nucleotide sequence which is capable of hybridising to a nucleotide sequence of the target polynucleotide that is adjacent to the nucleotide sequence that the second section of the second oligonucleotide is capable of hybridising to, said second section of the third oligonucleotide having a melting temperature (Tm) of between 40 and 70° C.; and
d) instructions for use.

In one embodiment the first oligonucleotide is labelled internally with at least one visually detectable label.

The kit of the invention may also comprise one or more selected from reaction buffer (for PCR or isothermal amplification), dNTPs, oligonucleotide primers, enzyme and further additives including but not limited to MgCl₂, Bovine Serum Albumin (BSA), Dimethyl Sulfoxide (DMSO), Betaine, Tween-20 and carrier RNA. Two or more of the elements making up the kit may be provided in a single mixture.

The parts making up a kit may be provided in a liquid form or as stabilised reagents (using a technique including but limited to lyophilisation and gelification) or a combination of the two.

In an eighth aspect of the invention there is provided an oligonucleotide library comprising a plurality of first oligonucleotides attached to a solid support, each first oligonucleotide comprising a first section, said first section comprising a nucleotide sequence that is labelled with at least one visually detectable label, and is capable of the nucleotide sequence being extended to include a second section, the second section of the first oligonucleotide being capable of hybridising to a target polynucleotide and the first section of the first oligonucleotide not being capable of hybridising to the nucleotide sequence of a target polynucleotide; and wherein the at least one visually detectable label undergoes a detectable change when the first oligonucleotides hybridise to both a target polynucleotide and a second oligonucleotide; said second oligonucleotide having a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to, said second section of the first oligonucleotide having a melting temperature (Tm) of between 25 and 50° C. and the second section of the second oligonucleotide having a melting temperature (Tm) of between 40 and 70° C.

In one embodiment, each oligonucleotide making up the plurality of oligonucleotides comprises a different visually detectable label.

Preferably, the first oligonucleotide is labelled internally with the at least one visually detectable label.

In one embodiment of the library the solid support is a polynucleotide synthesis resin. The synthesis resin is therefore one on which polynucleotides can be synthesised by the addition of nucleotide units (e.g. monomers) in order to synthesise a polynucleotide of desired sequence and length. Such methods and resins are disclosed in Matteucci & Caruthers (1981).

Alternatively, the library of first oligonucleotide first section (universal element) sequences may be large-scale syntheses of chemically-modified oligonucleotides which are purified and stored for subsequent attachment to separately synthesised first oligonucleotide second section (target specific) oligonucleotides using click-chemistry methods, such as SPAAC (Shelbourne et al. 2011), CuAAC (Kumar et al. 2007) and Dies-Alder (El-Sagheer et al. 2011) reactions. The ‘click’ attachment of first and second sections generates the complete first oligonucleotide.

The visually detectable label may be a fluorophore or a dye, for example fluorescein dT.

In one embodiment of the library, the first section of the first oligonucleotide is labelled with two or more; three or more; or four or more visually detectable labels.

All of the third to eighth aspects of the invention may be further defined in relation to the oligonucleotides as follows.

In one embodiment, the first and second sections of the first oligonucleotide are joined at a junction, said junction being positioned at least two nucleotides from the visually detectable label positioned 5′-most when the first oligonucleotide has been synthesised in the 3′ to 5′ direction; or said junction being positioned at least two nucleotides from the visually detectable label positioned 3′-most when the first oligonucleotide has been synthesised in the 5′ to 3′ direction. In alternative embodiments, the at least one visually detectable label is positioned at least three, or at least four nucleotides from the junction. In a further embodiment, the at least one visually detectable label is positioned between two and four, i.e. two, three or four, nucleotides from the junction.

As a further alternative, the first and second sections of the first oligonucleotide are joined at a junction, said junction being positioned one nucleotide from the visually detectable label positioned 5′-most when the first oligonucleotide has been synthesised in the 3′ to 5′ direction; or said junction being positioned at one nucleotide from the visually detectable label positioned 3′-most when the first oligonucleotide has been synthesised in the 5′ to 3′ direction.

In one embodiment, the independent partner oligonucleotides are not labelled with a quencher.

### EXAMPLES AND FIGURES

The invention will now be described in more detail with reference to the following figures and examples.

The invention is demonstrated through a number of examples. These show:
1. The detection of different target sequences in a three-strand ‘tribrid’ configuration either as reverse complements or as a PCR or isothermal amplification product.
2. The detection of different target sequences in a four-strand ‘cruciform’ configuration.
3. The ability to create either fluorescent enhancing hybridisation events, or fluorescent quenching hybridisation events depending on the molecular sequence of the universal element used.
4. The use of different ‘universal’ element sequences indicating a lack of, or minimal sequence constraints.
5. The finding that a particular universal element creates a reproducibly fluorescence enhancing response to hybridisation, or a reproducibly fluorescent quenching response to hybridisation, such that a library of such elements can be created as predictable precursors for future probes
6. The same universal element can be used in different probes which can be multiplexed and function effectively independent of each other in the same reaction.
7. The combination of the junction probe and the universal complementary element oligonucleotide/s in the absence of target sequence may hybridise to cause fluorescence changes but only at temperatures below that which are diagnostic of the presence of the target DNA.
8. The junction probe will not generate hybridisation fluorescence changes in the absence of the universal complementary oligonucleotide but in the presence of the target DNA at temperatures which are diagnostic of the target sequence.
9. The tribrid and cruciform structures can function with the use of multiple fluorophores and different fluorophores.
10. The tribrid structures can detect variations within the target sequence that is complementary to the junction probe, but also within the target sequence that is complementary to the universal complement oligonucleotide.
11. The junction probe can be configured such that it can be present in amplification reactions but does not require the 3′ terminal to be blocked.
12. Both the junction probe and the universal complementary oligonucleotide can contain fluorescent active moieties which interact in the universal sequence region.

### FIGURE LEGENDS

FIG. 1. Target sequence detection using a junction probe and universal complement oligonucleotide. The junction probe comprises universal (1) and target specific elements (2). The universal complement oligonucleotide comprises a sequence complementary to the probe universal element (3) and a target specific sequence (4). Target detection requires probe hybridisation to both the universal complement oligonucleotide and the target sequence to form a tribrid junction structure. Formation of the tribrid structure causes either (A) fluorescence enhancement with fluorophores positioned further away from the quenching DNA or (B) fluorescence quenching with fluorophores coming into closer contact with DNA bases. Fluorescence enhancement or quenching upon tribrid formation depends on the sequence of the universal element and location of the dye-labelled bases. At higher temperatures fluorescence emission return to the levels observed with single-stranded junction probes. Single nucleotide polymorphisms (*) located in the target sequences of junction probe and/or universal complement oligonucleotides reduce the stability of tribrid structures allowing sequence identification through melting and annealing curve analysis.

FIG. 2. The two-phase method used for synthesis of universal probes. The first phase comprises repeated cycles of detritylation, activation & coupling and oxidation to generate the universal element sequence using a large synthesis scale. The resin is then divided and used for synthesis of multiple smaller scale oligonucleotides by adding target specific sequence onto the universal elements.

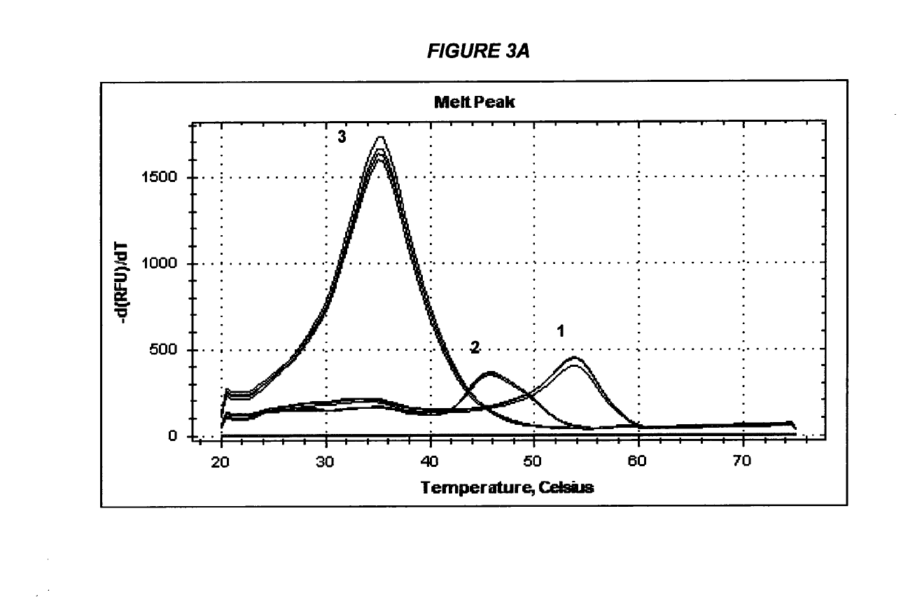

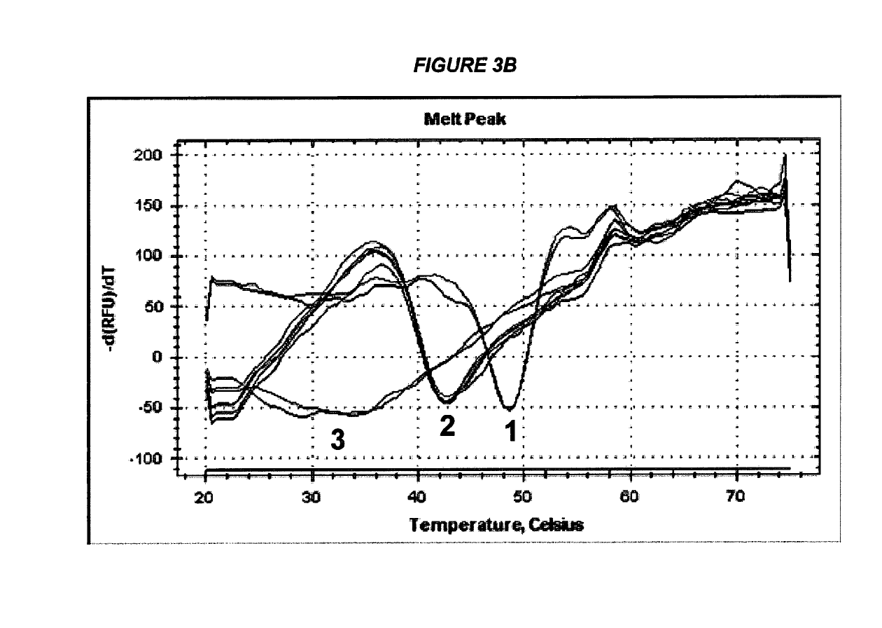

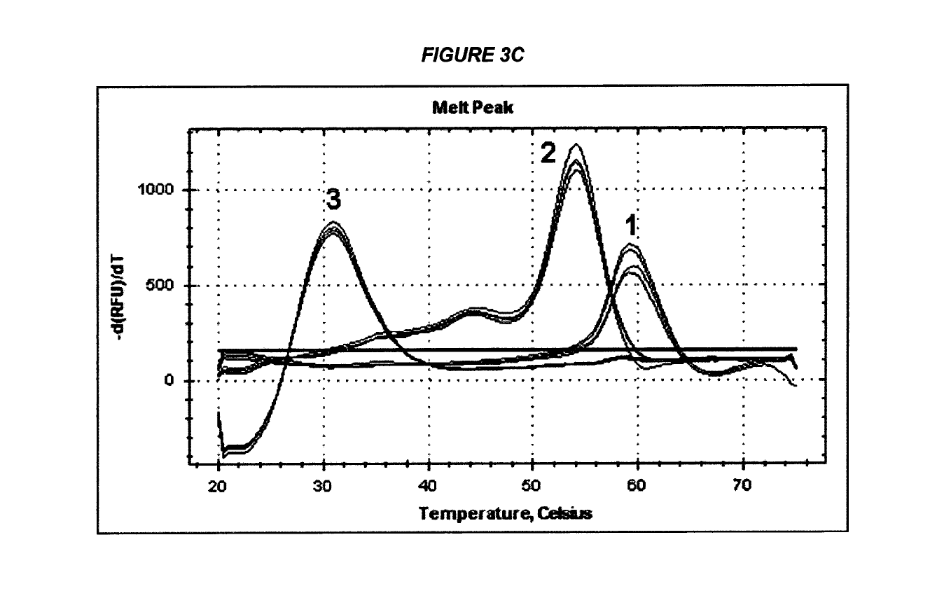

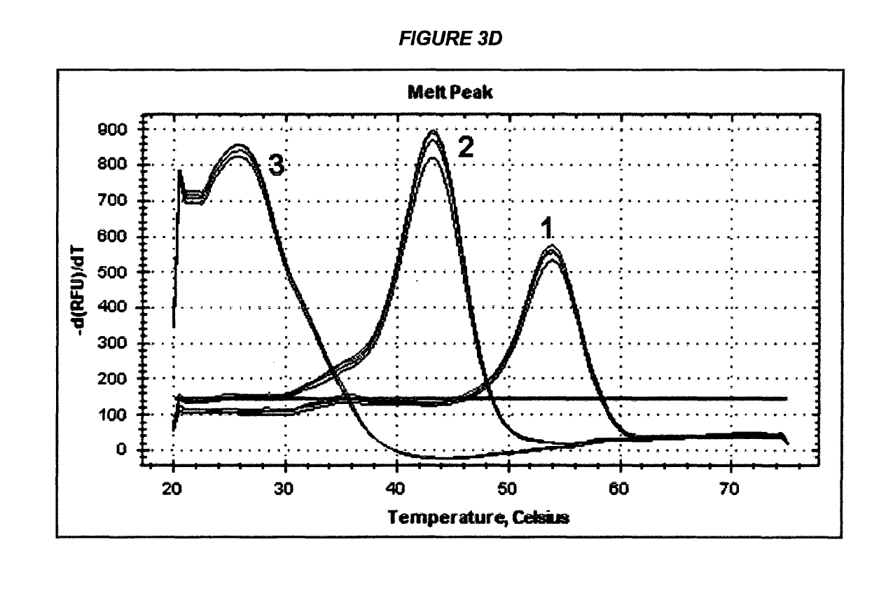

FIG. 3. Oligonucleotide melting curve analysis with A) HRH2-JP, B) CDK-JP, C) AIH7-JP and D) CXC-JP probes. Melting curve analysis was performed with 1) fully complementary target sequence, 2) mismatched target sequence and 3) in the absence of target sequence.

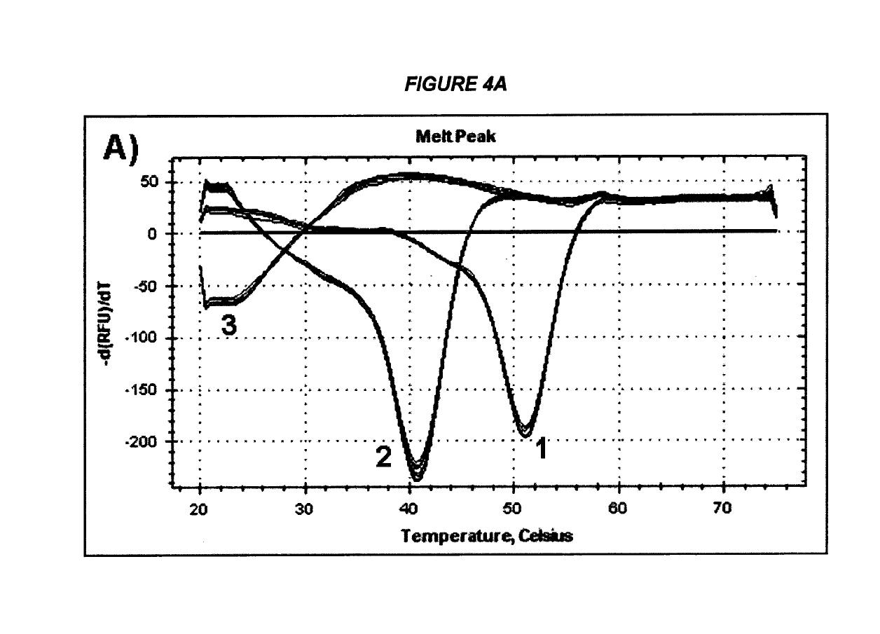

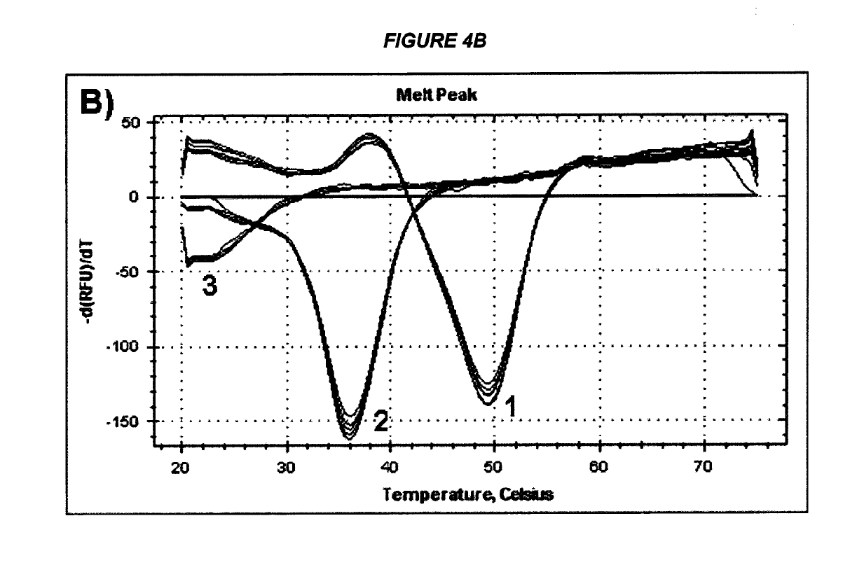

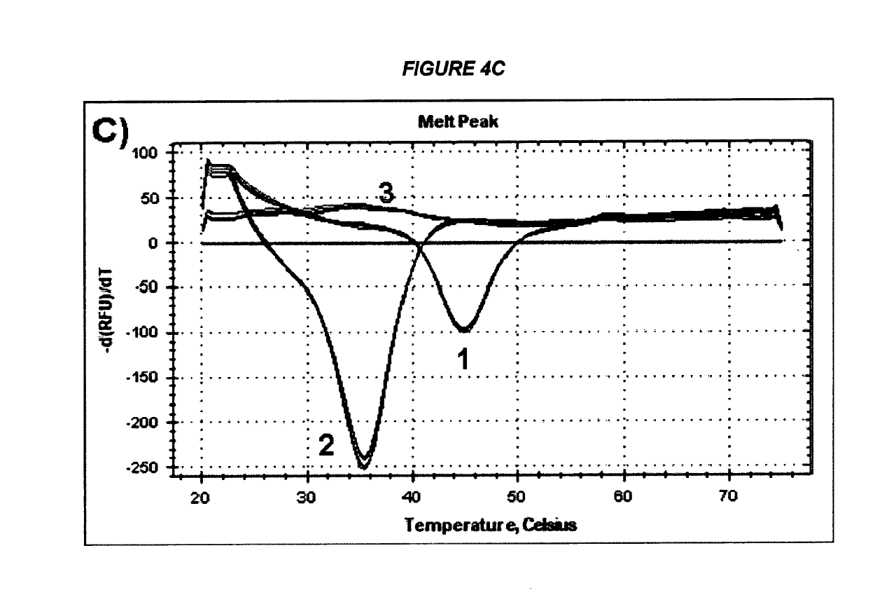

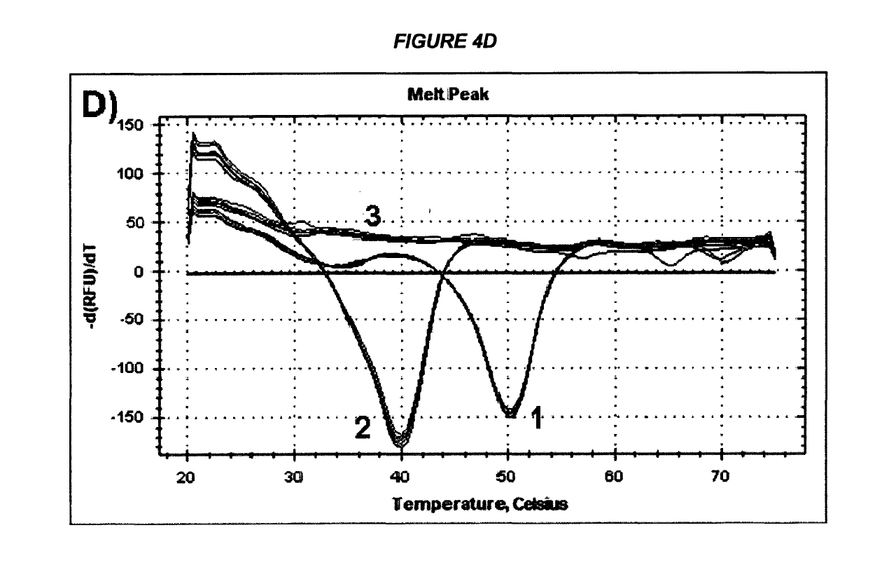

FIG. 4. Oligonucleotide melting curve analysis with A) G1-JP, B) P2-JP, C) CDK2-JP and D) CH-JP probes. Melting curve analysis was performed with 1) fully complementary target sequence, 2) mismatched target sequence and 3) in the absence of target sequence.

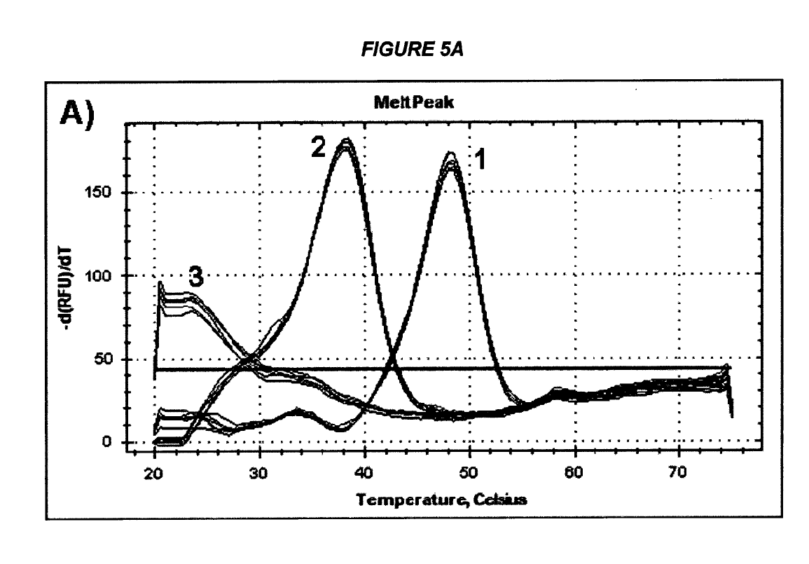

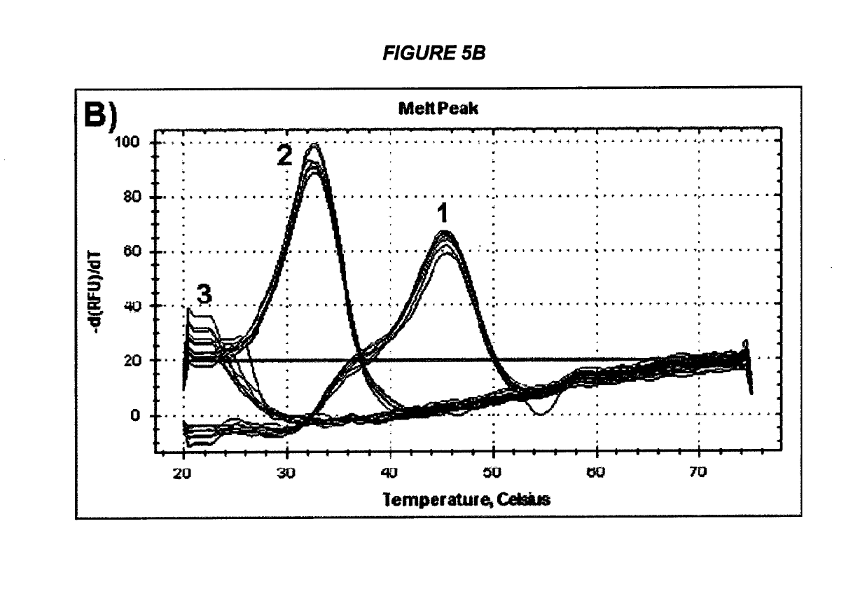

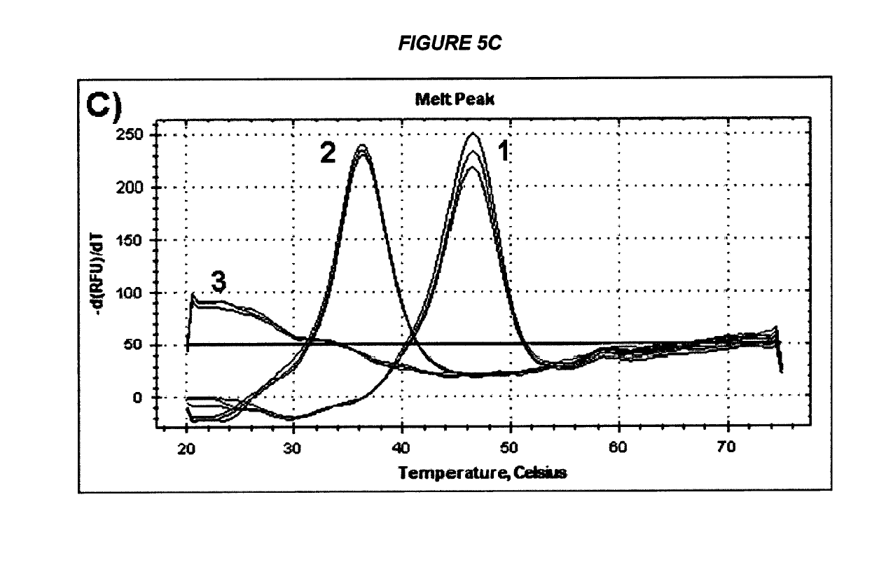

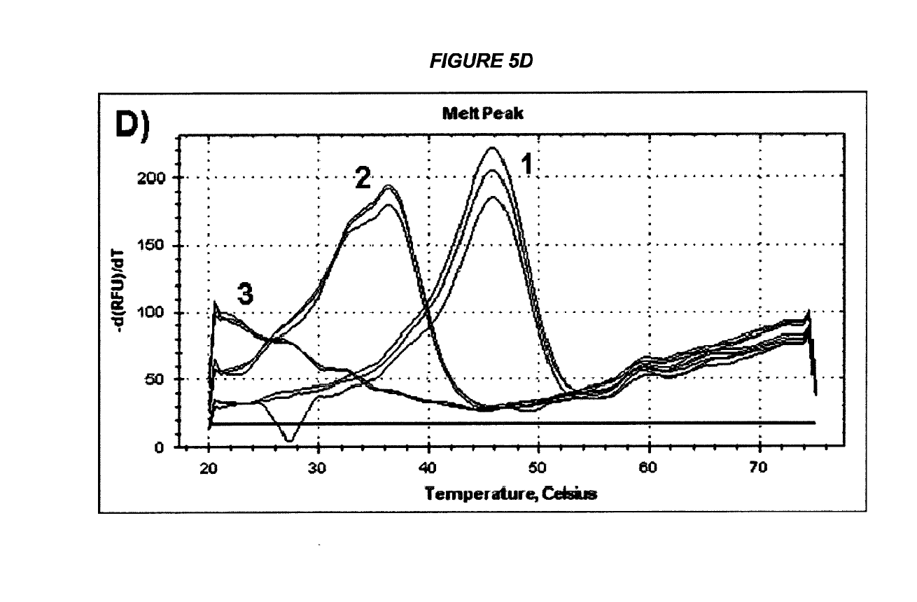

FIG. 5. Melting curve analysis with A) 75 nM G1-JPv2+500 nM G1-UCv2, B) 75 nM P2-JPv2+500 nM P2-UCv2 and C) 150 nM G1-JPv2+150 nM P2UCv2 oligonucleotides. Annealing curve analysis D) was performed with 150 nM G1-JPv2+150 nM P2UCv2. Melting and annealing curve analysis was performed with 1) fully complementary target sequence, 2) mismatched target sequence and 3) in the absence of target sequence.

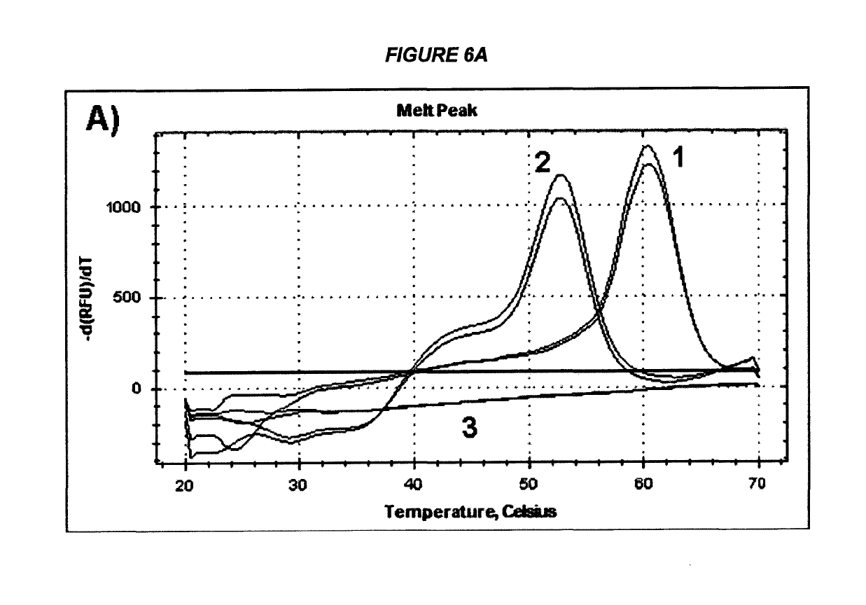

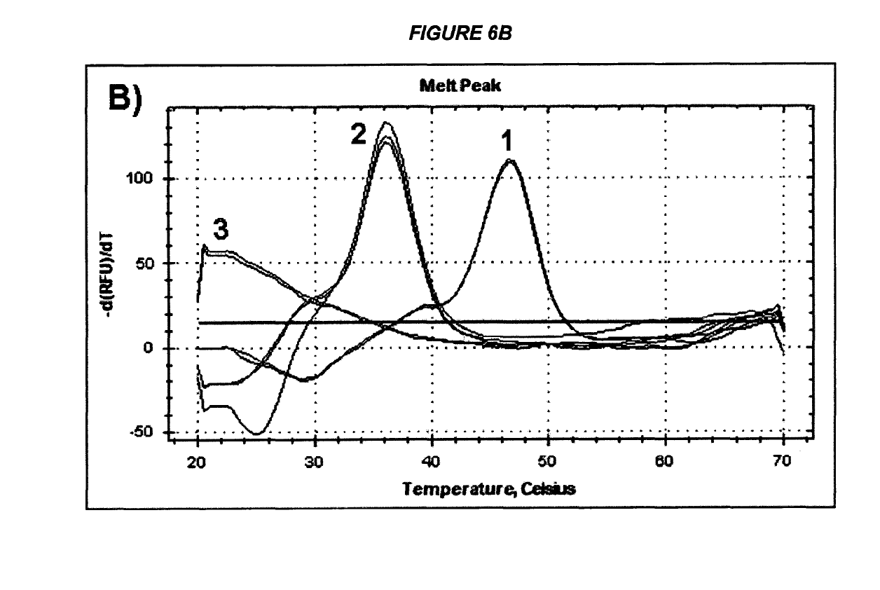

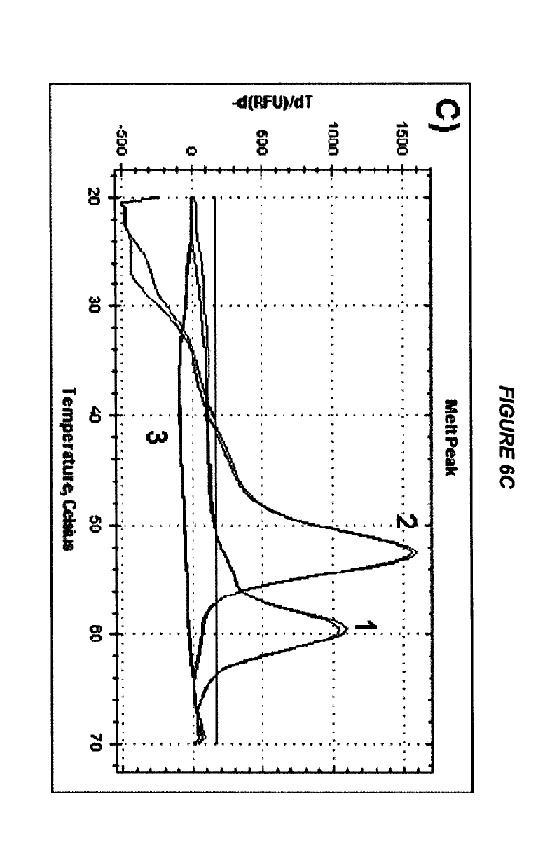

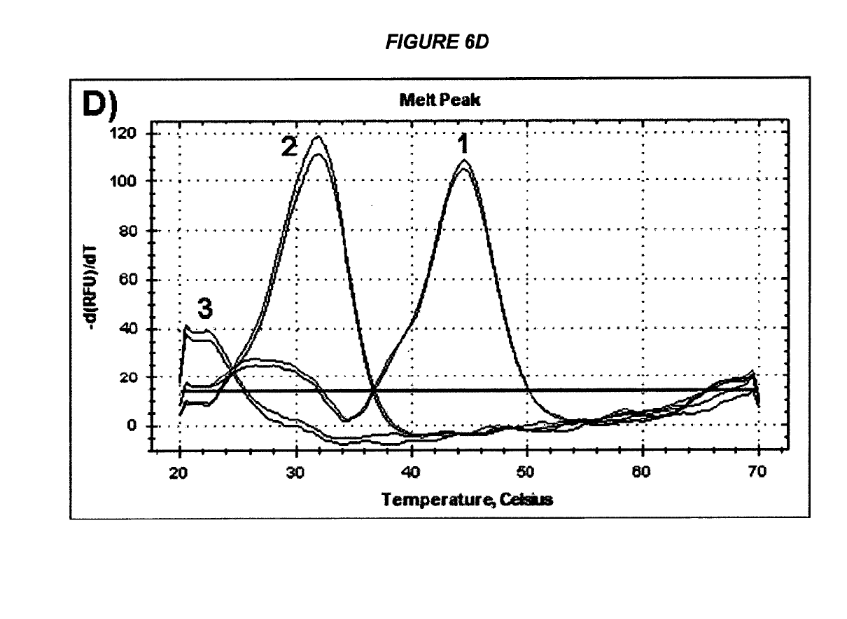

FIG. 6. Melting curve analysis using PCR amplified target sequences. Amplified targets were detected using A) the standard HyBeacon® probe G1-L, B) the junction probe G1-JPv2, C) the standard HyBeacon® P2-L and D) the junction probe P2-JPv2. Melting peaks were generated with 1) fully complementary and 2) mismatched target sequences. Melting curve analysis in the absence of target sequence 3) is presented for comparison.

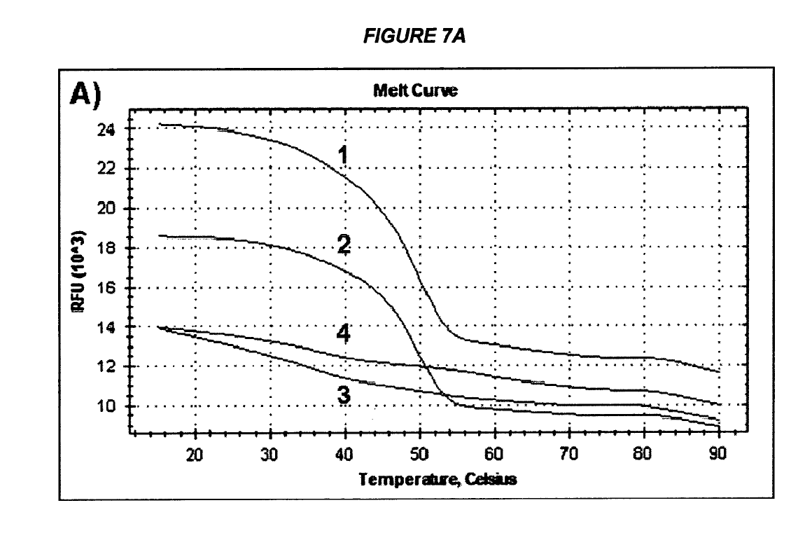

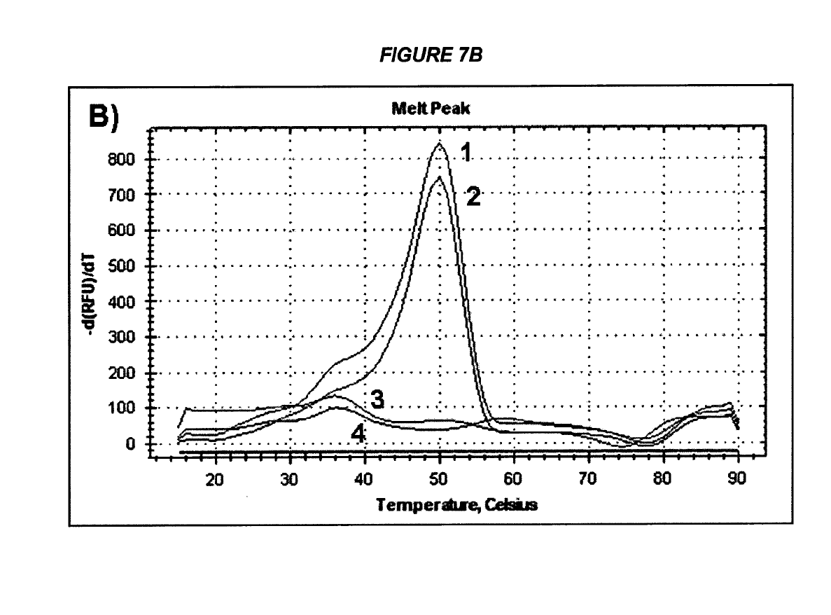

FIG. 7. Melting curve (A) and melting peak (B) analysis of CH-JPv2-UE1 and CH-JPv2-UE2 probes. Melting peaks were generated with the CH-T1 target sequence using 1) the CH-JPv2-UE2 probe and CH-UCv2-UE2 universal complement oligonucleotide and 2) the CH-JPv2-UE1 probe and CH-UCv2-UE1 universal complement oligonucleotide. Target sequence is not detected when the universal element sequences do not match such that melting peaks are not generated when 3) the CH-JPv2-UE1 probe is used with CH-UCv2-UE2 and 4) the CH-JPv2-UE2 probe is used with CH-UCv2-UE1.

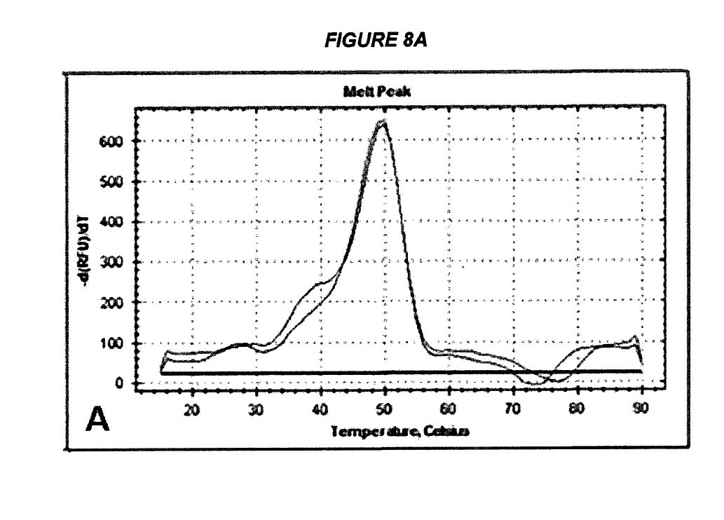

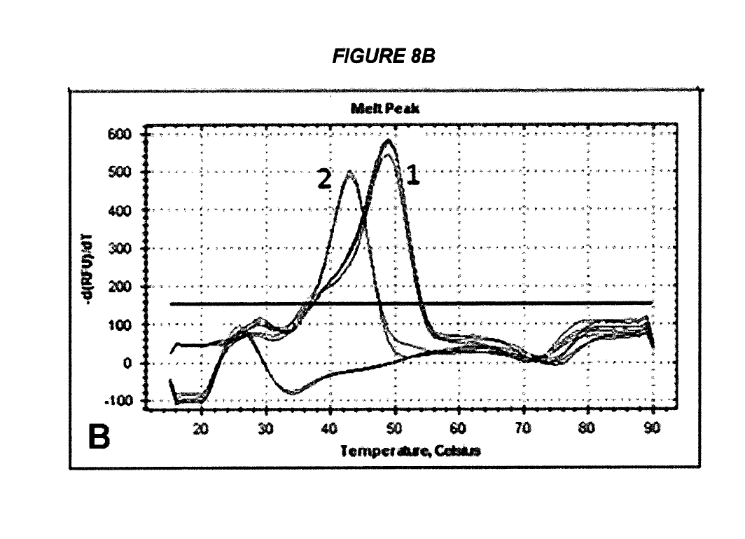

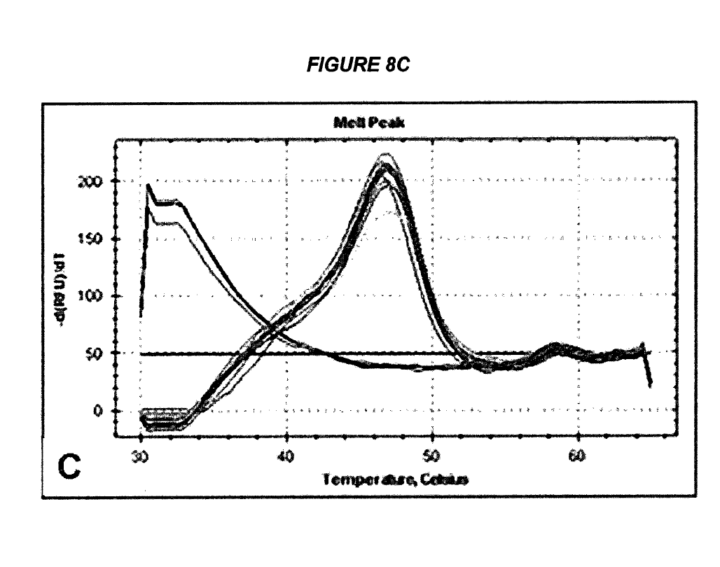

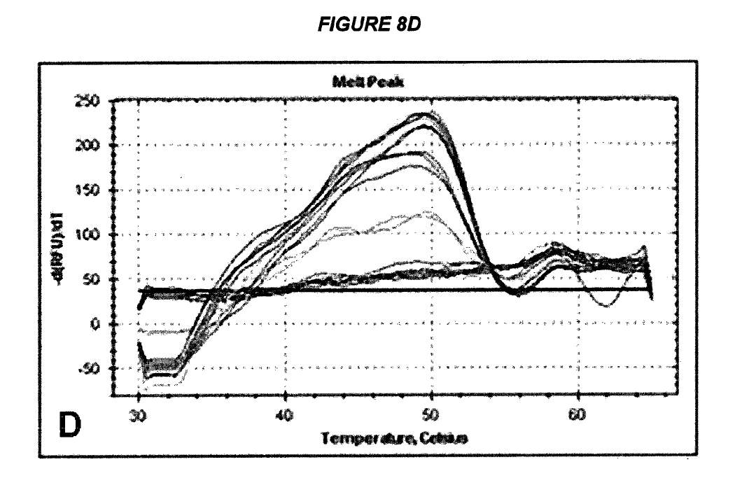

FIG. 8. (A) Melting peak analysis using the SIMA-labelled junction probe CH-JPv2-SIMA. Multiplex analysis (B) using 1) CH-JPv2-SIMA and 2) JP-22 probes allows simultaneous detection of CH-T3 and T-22 target sequences with SIMA-dT and fluorescein-dT dye labels, respectively. Multiplex PCR allows simultaneous detection of Chlamydia trachomatis plasmid and genomic DNA target sequences using the (C) fluorescein-labelled CH-JPv2-UE1 and (D) SIMA-labelled S1-JP probes. Multiplex target detection was successfully achieved down to 1.8 genomic copies.

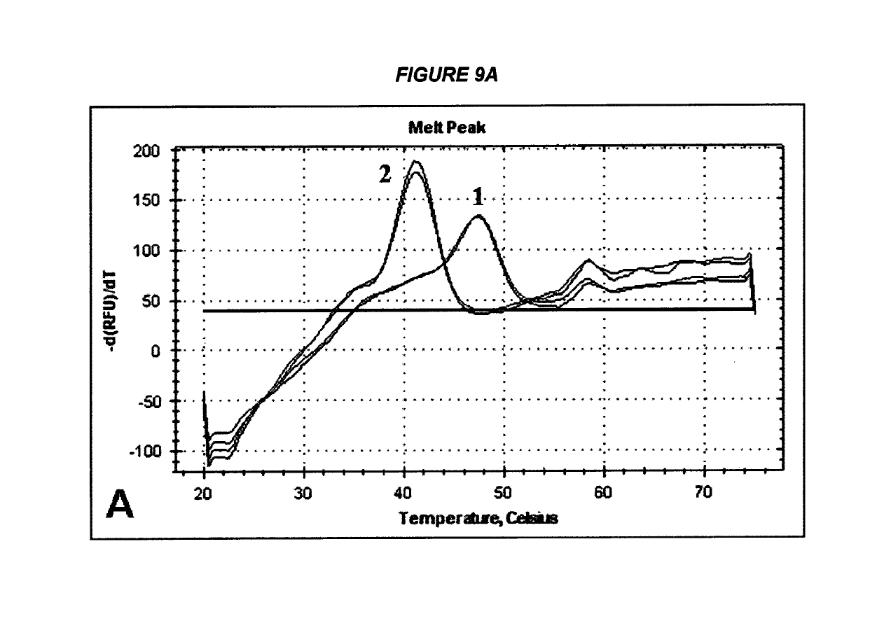

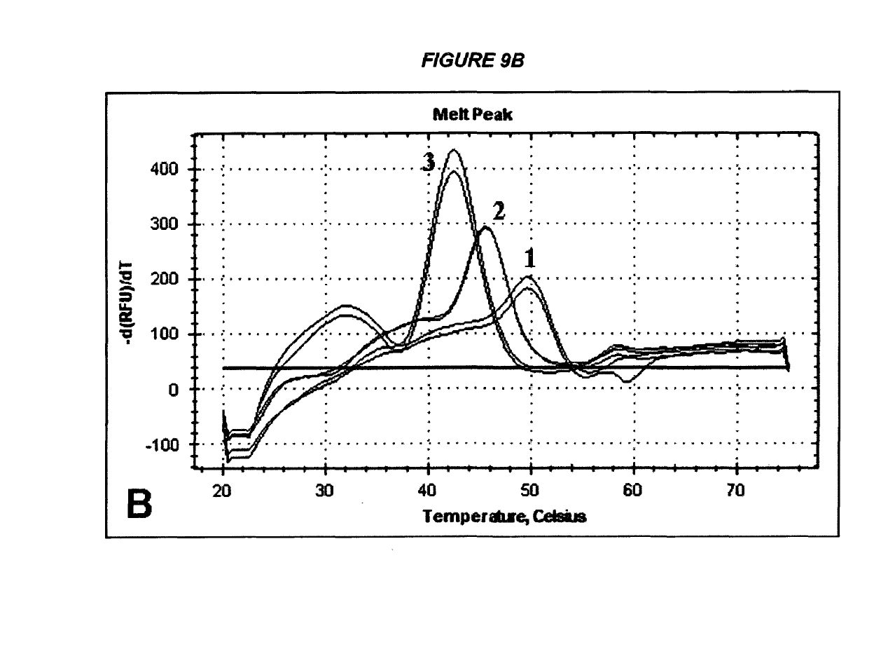

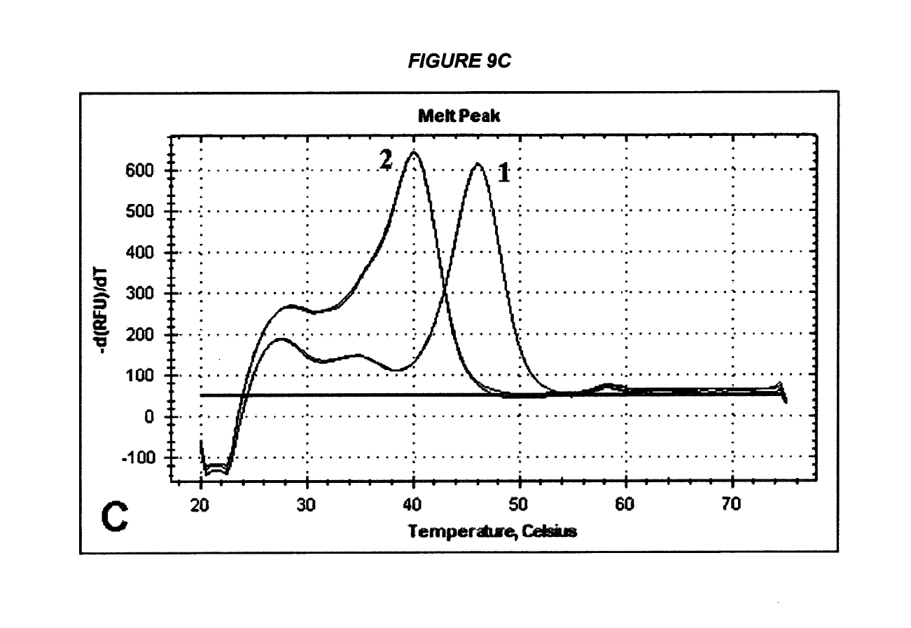

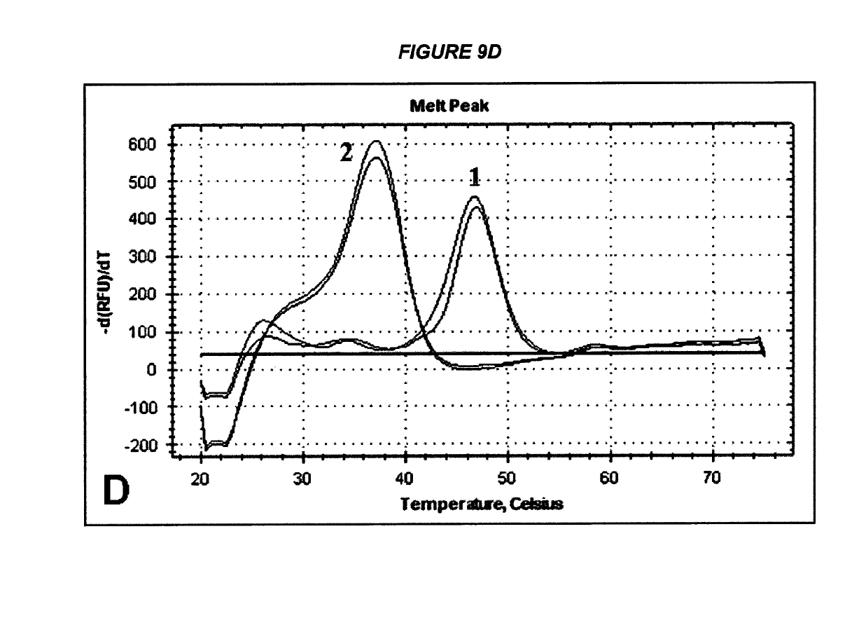

FIG. 9. Melting peak analysis using (A) JP-1 & UC-1, (B) JP-2 & UC-2, (C) JP-3 & UC-3 and (D) JP-4 & UC-4 oligonucleotides to detect and discriminate polymorphic target sequences. Melting peaks indicate the presence of 1) fully complementary target sequence, 2) target sequence containing a SNP or insertion/deletion polymorphisms or 3) two different SNPs in the same target sequence.

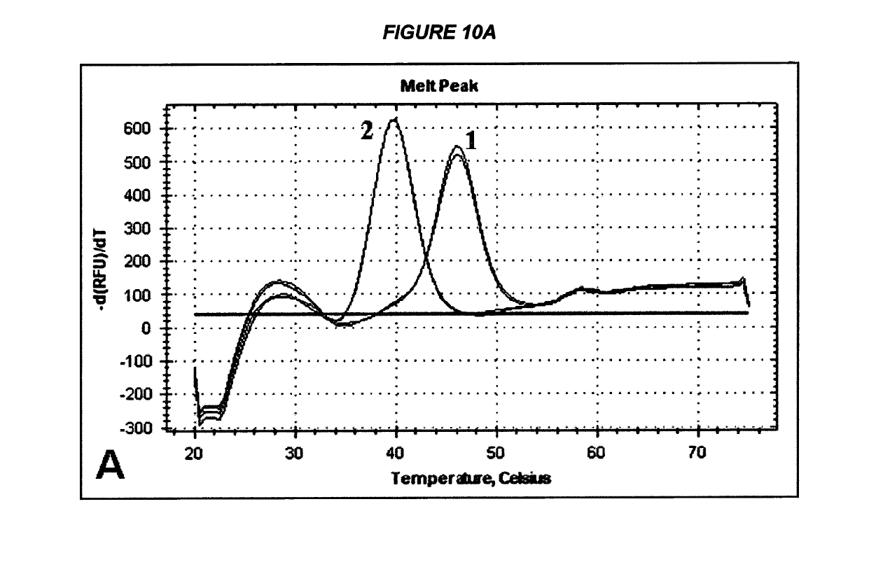

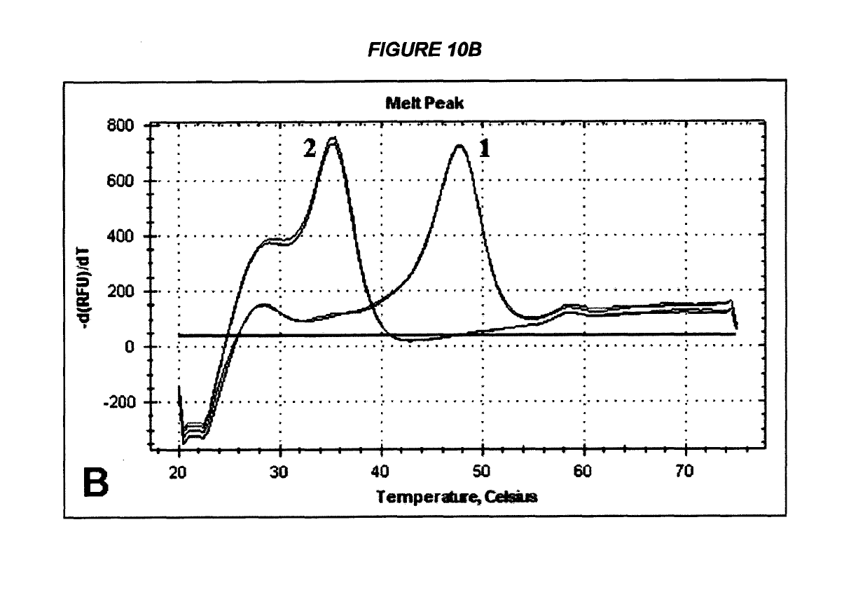

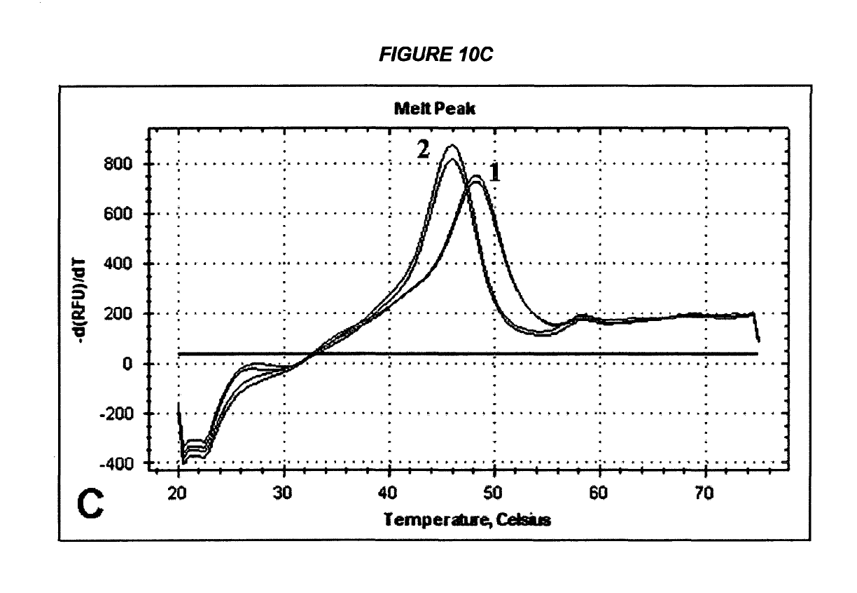

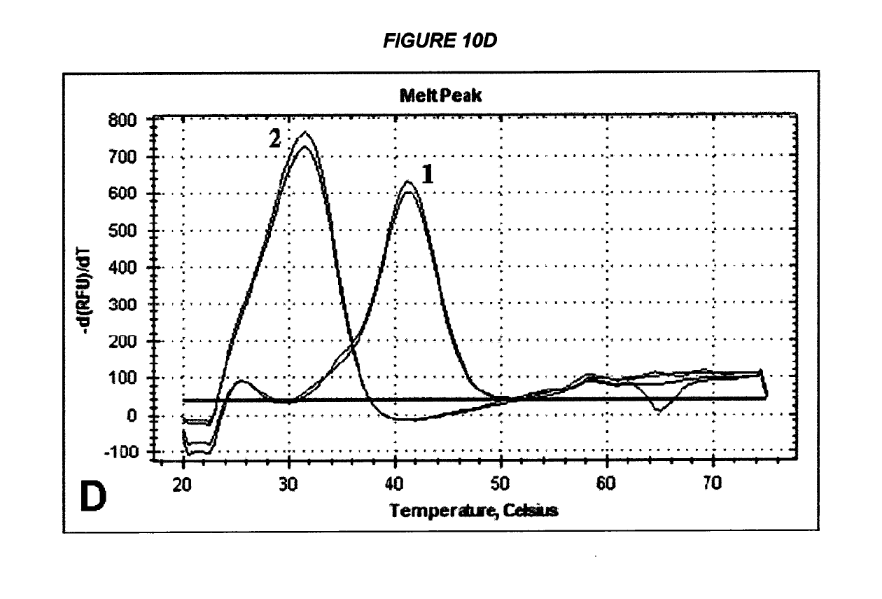

FIG. 10. Melting peak analysis using (A) JP-5 & UC-5, (B) JP-6 & UC-6, (C) JP-7 & UC-7 and (D) JP-8 & UC-8 oligonucleotides to detect and discriminate polymorphic target sequences. Melting peaks indicate the presence of 1) fully complementary target sequence or 2) target sequence containing a SNP or insertion/deletion polymorphisms.

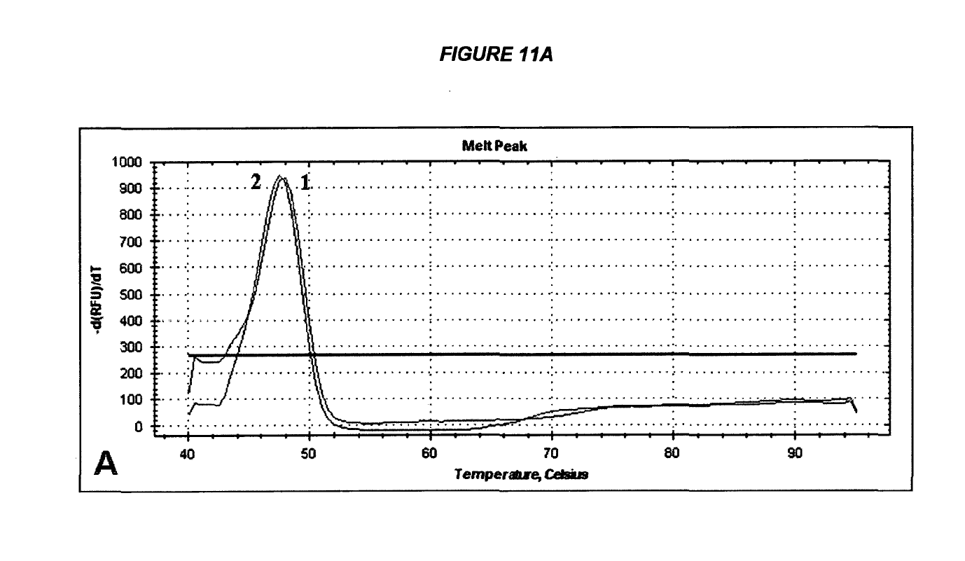

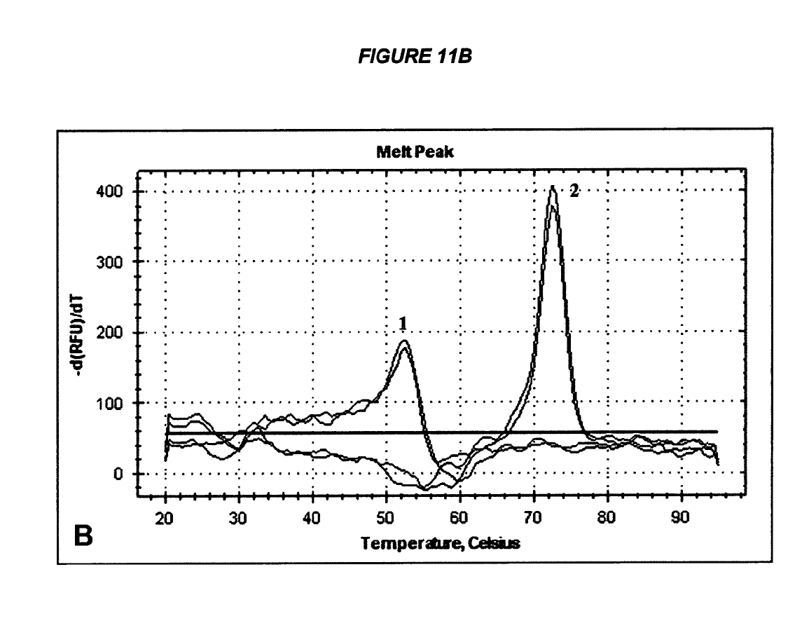

FIG. 11. Investigation of probe extension in the absence of a 3′ PCR blocking modification. (A) Melting peak analysis using the CH-UCv2-UE1 universal complement oligonucleotide with 1) the CH-JPv2-UE1 probe possessing a 3′ phosphate and 2) the CH-JPv2-UE1np probe lacking a 3′ PCR blocker. Both probes generate a single target melting peak with no evidence of probe extension. B) Melting curve analysis with the S1-JP probe and S1-UC oligonucleotides yields 1) a 52° C. peak with PCR positive samples or 2) a 72° C. peak with NTCs arising from dimer formation.

FIG. 12. Detection of target sequences using Loop-Mediated Isothermal Amplification (LAMP). Melting peaks are generated 1) in the presence of target DNA but not with 2) No Template Controls (NTCs).

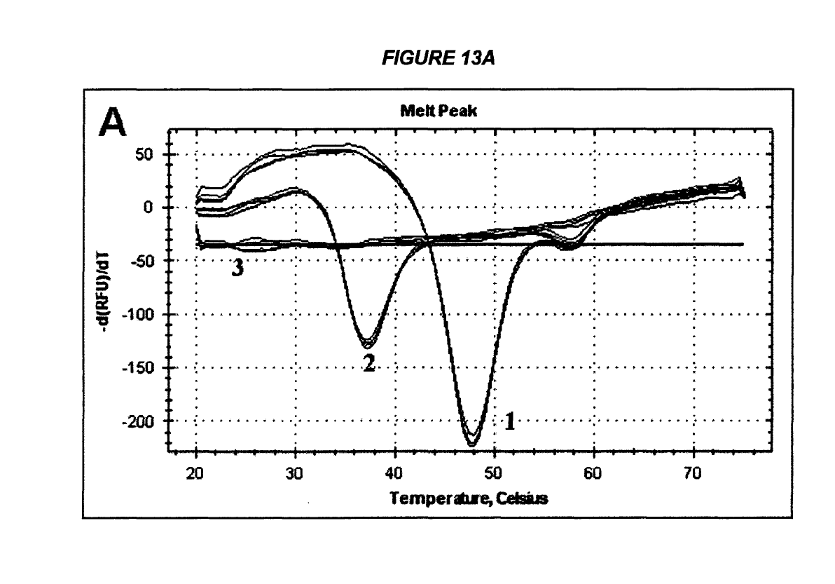

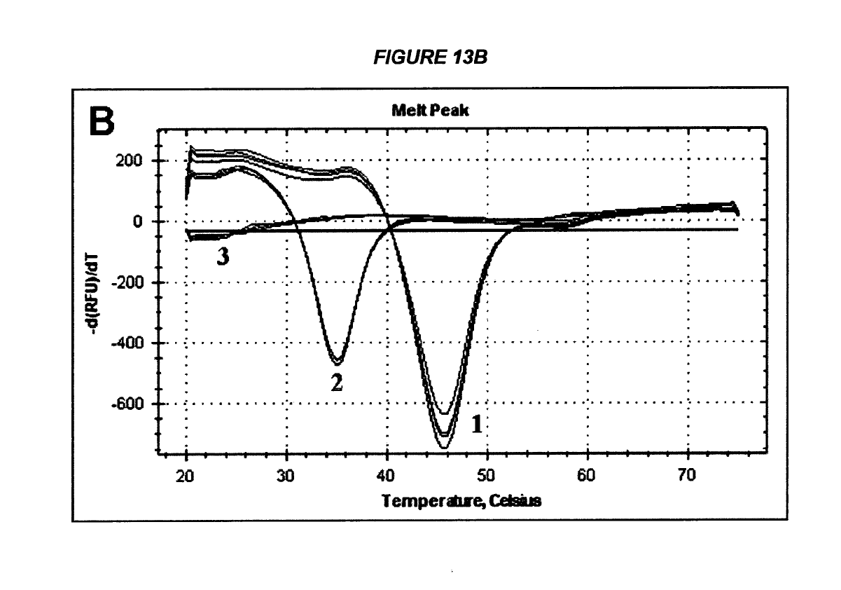

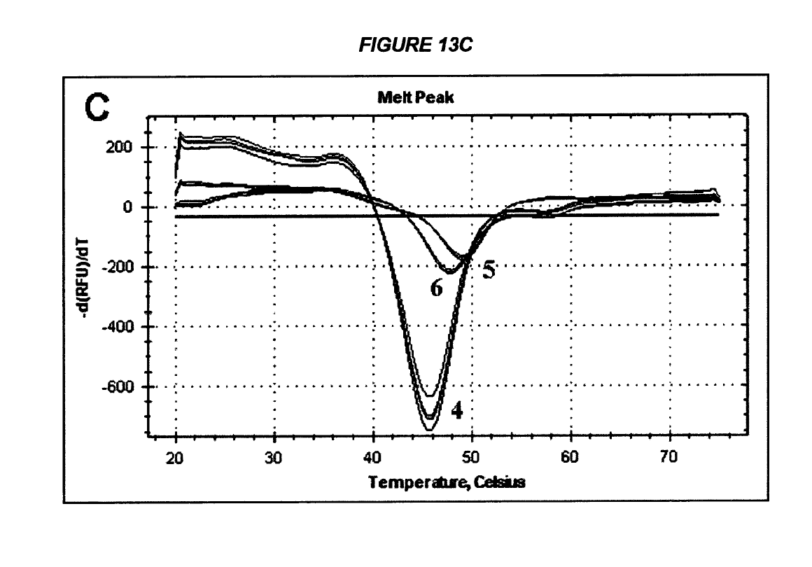

FIG. 13. Melting curve analysis of A) the labelled G1-JPrc junction probe and unlabelled G1-UCrc complement oligonucleotide and B) the G1-JP and G1-JPrc probes where both universal element strands were labelled. Melting curve analysis was performed in the presence of 1) fully complementary G1-T1 target, 2) mismatched G1-T2 target and 3) in the absence of target sequence. A comparison of tribrid structures C) demonstrates that the melting peaks generated with 4) G1-JP+G1-JPrc (both universal element strands labelled) are larger than the sum of 5) G1-JP+G1-UC and 6) G1-JPrc+G1-UCrc (where only one of the universal element strands contains fluorophore labelled bases).

FIG. 14. A four-strand cruciform structure formed with 1) the P2-JPv2 probe, 2) G1-UCv2 complement oligonucleotide, 3) CRUX-UC complement oligonucleotide and 4) G1-T1 target sequence. The positions of fluorophore-labelled nucleotides are indicated.

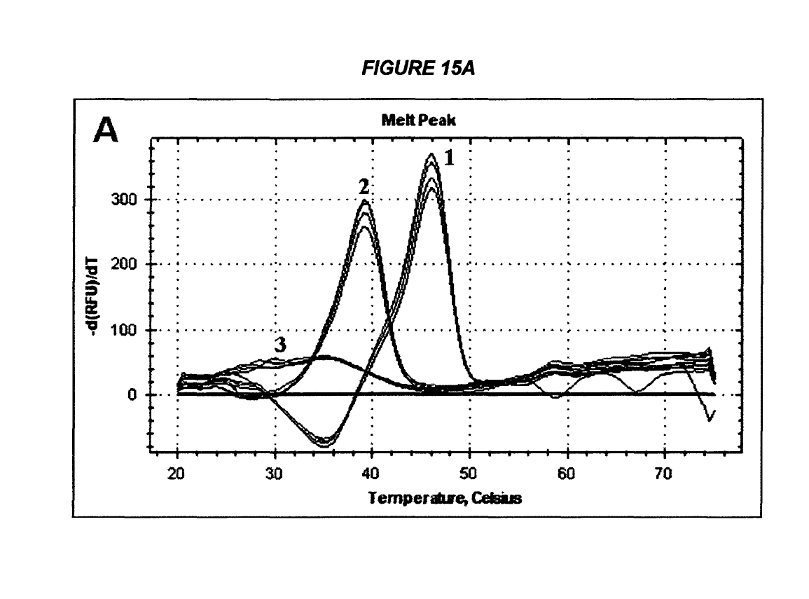

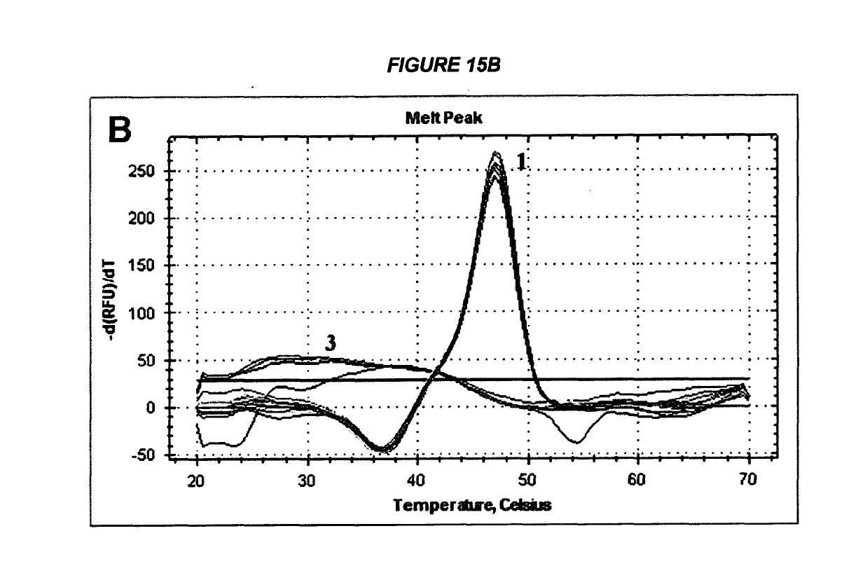

FIG. 15. Detection and identification of 1) the G1-T1 and 2) G1-T2 target sequences using the four-strand structure formed with P2-JPv2 probe and the G1-UCv2 and CRUX-UC complement oligonucleotides. A no target control 3) is presented for comparison. Detection is demonstrated with A) oligonucleotide and B) PCR-amplified target sequences.

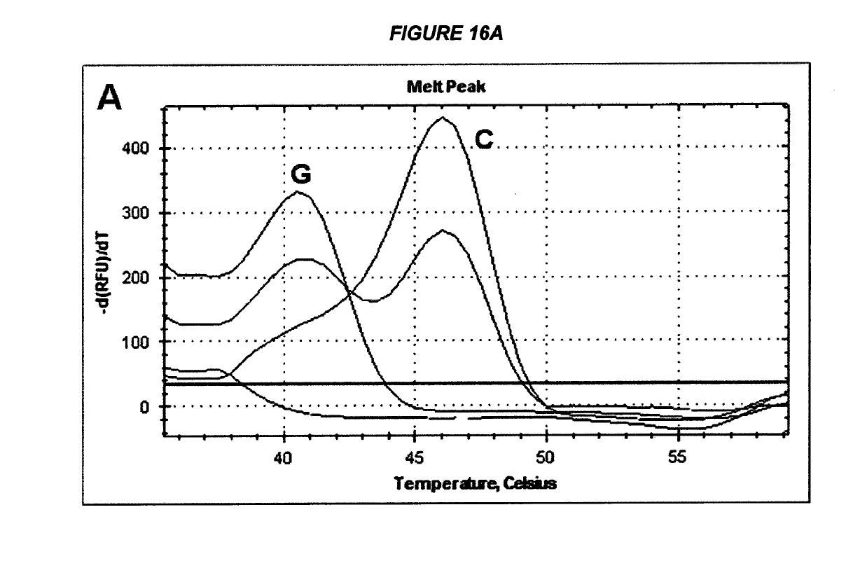

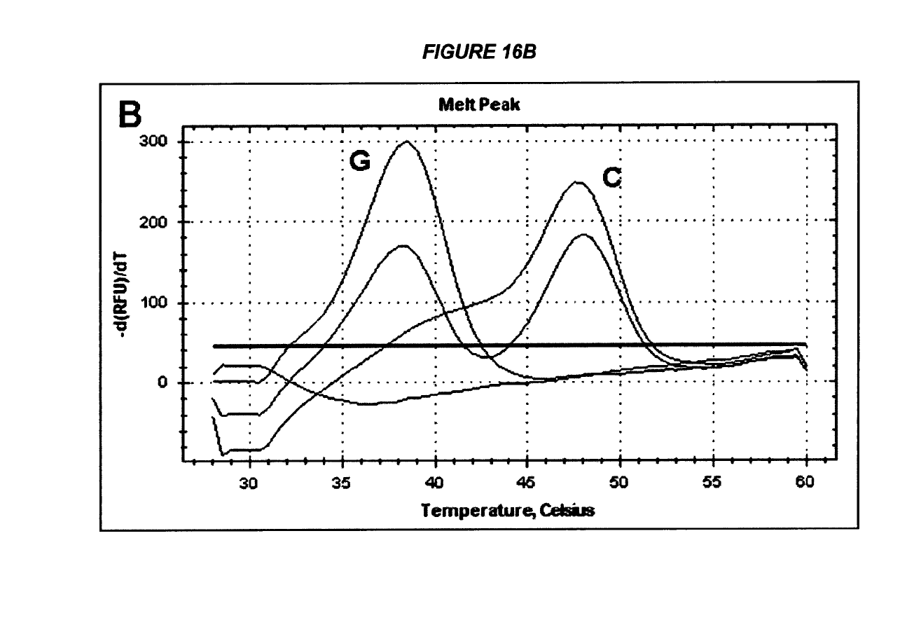

FIG. 16. Analysis of the CYP2D6 rs1058164 SNP using A) four-strand cruciform and B) three-strand tribrid structures to detect and differentiate the G and C allele variants.

FIG. 17. Melting curve analysis of A) the tribrid structure formed with G1-JPv2 probe, G1-UCv2 and target sequence and B) the four-strand structure formed with the P2-JPv2 probe, G1-UCv2 complement oligonucleotide, CRUX-UC complement oligonucleotide and target sequence. The effect of intervening target sequence was investigated using 1) G1-T1, 2) G1-1nt, 3) G1-2nt, 4) G1-3nt, 5) G1-5nt, 6) G1-10nt and 7) G1-20nt oligonucleotides.

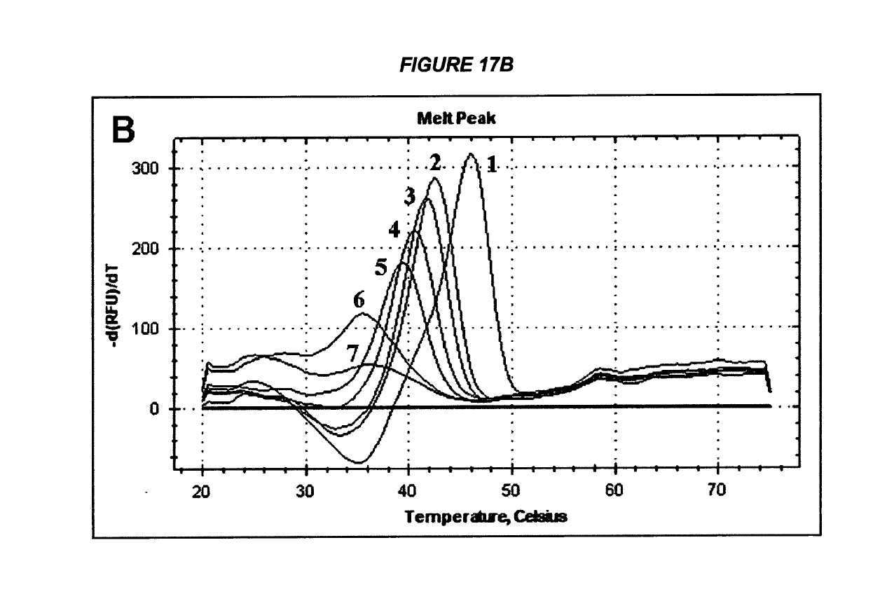

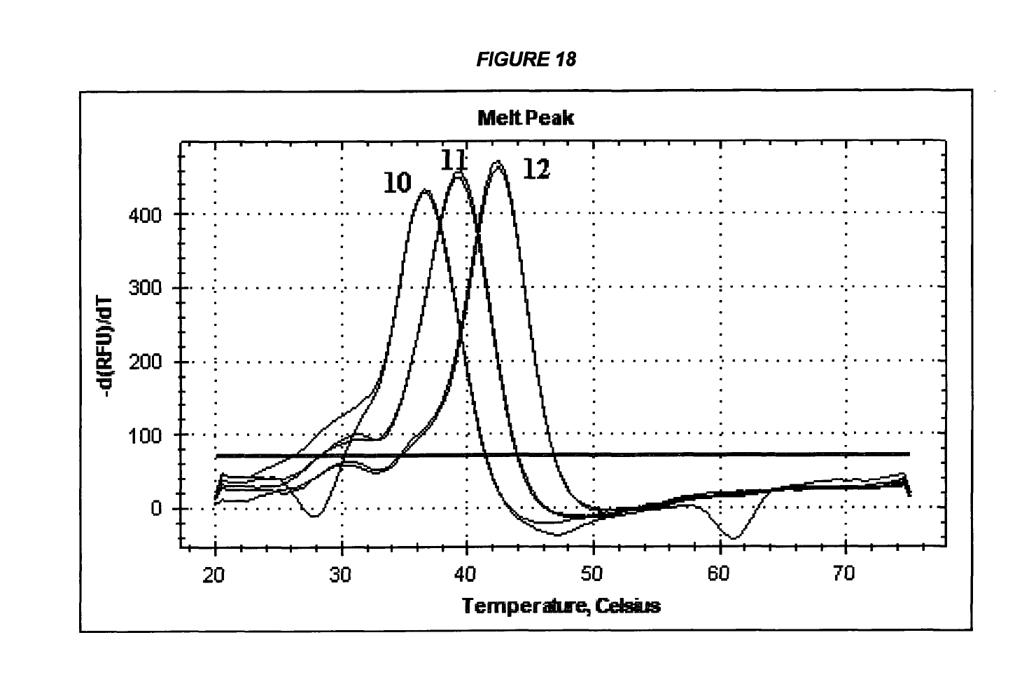

FIG. 18. Analysis of D16S539 STR length polymorphism using the P2-JPv2 probe with a four-strand cruciform design.

FIG. 19. A unimolecular approach to achieving universal sequence analysis using A) three-strand tribrid and B) four-strand cruciform methods. The junction probe (1) hybridises with UC oligonucleotide universal elements in the presence of target sequence. The universal complement oligonucleotide (2) is attached to the 5′ end of a primer via a spacer such as hexaethylene glycol (HEG) and hybridises with amplified target sequence as part of the same DNA strand (unimolecular binding). The four-strand cruciform approach uses a second UC oligo (3) that hybridises downstream on the target sequence.

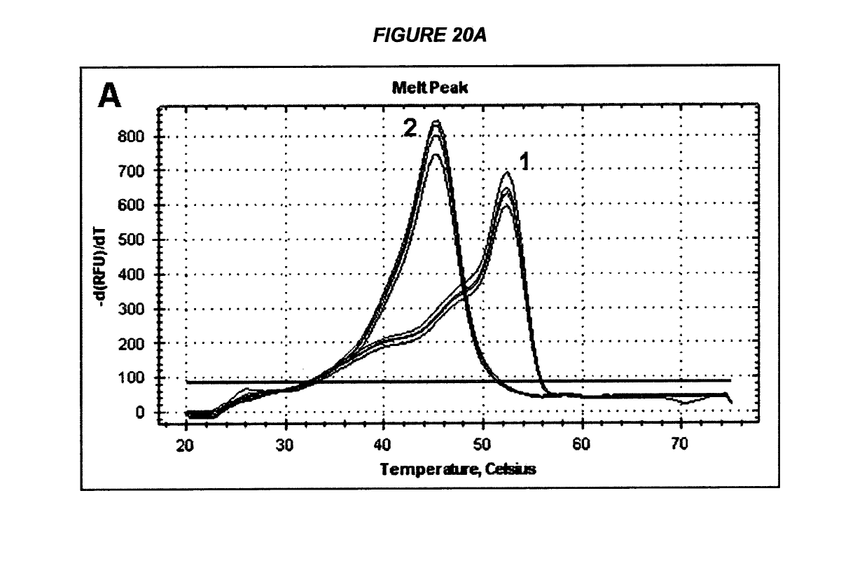

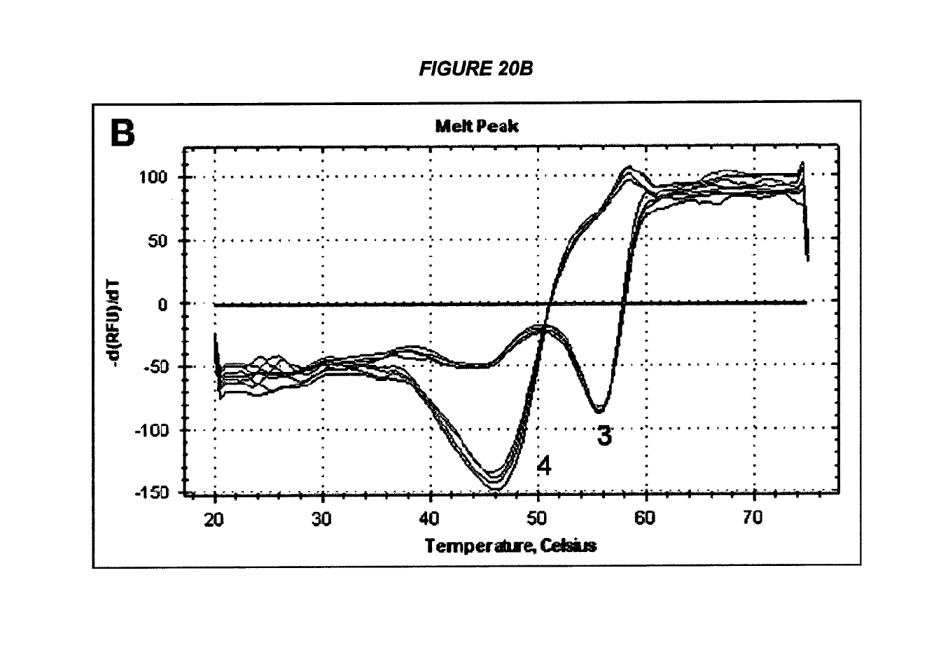

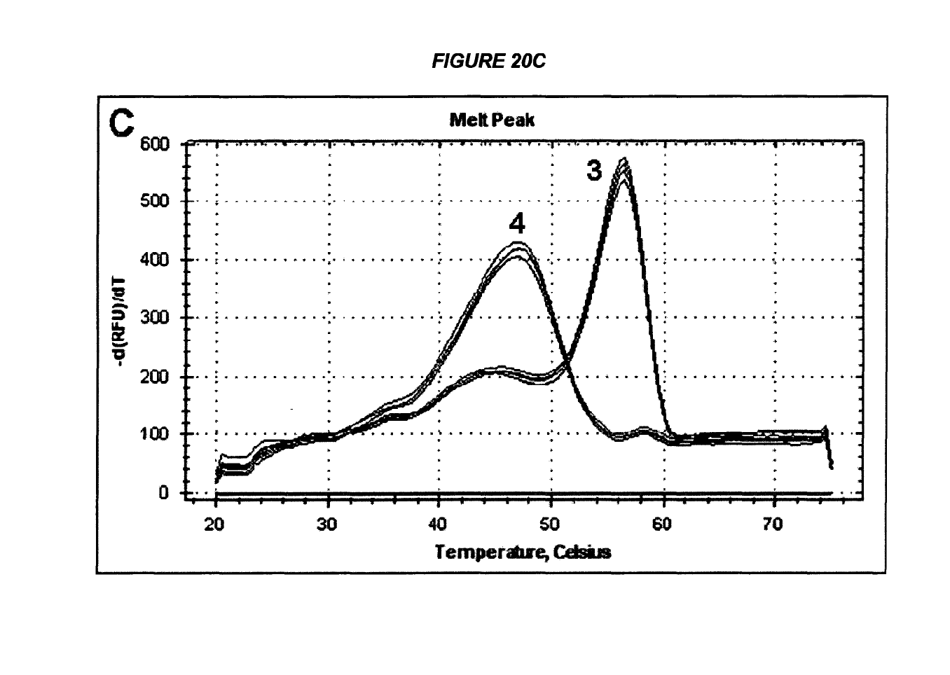

FIG. 20. Universal sequence analysis using probes labelled on 5′ or 3′ oligonucleotide termini. A) The P2-5JP was used in a four-strand structure to detect matched and mismatched target sequences. B) The P2-3JP probe yields inverted peaks in a three-strand structure when the universal element sequences of probe and UC are equal length. C) P2-3JP yields positive peaks when the length of the universal element in the UC oligonucleotide exceeds the length of the probe. The oligonucleotide targets G1-T1 (1), G1-T2 (2), P2-T1 (3) and P2-T2 (4) were used for melting curve analysis.

FIG. 21. Simplified diagram of probe.

### MATERIALS AND METHODS FOR THE EXAMPLES

### Oligonucleotide Probe Design and Synthesis

Standard DNA phosphoramidites, solid supports and additional reagents were purchased from Link Technologies or Applied Biosystems Ltd. All oligonucleotides were synthesised on an Applied Biosystems 394 automated DNA/RNA synthesiser using a 0.2 μmole phosphoramidite cycle of acid-catalysed detritylation, coupling, capping and iodine oxidation. Normal monomers (A, G, C and T) were allowed to couple for 25 seconds and all other monomers for an additional 300 seconds. Stepwise coupling efficiencies and overall yields of monomers with DMT protection were determined by measuring trityl cation conductivity and in all cases were >98.0%. Cleavage of the oligonucleotides from the solid support was carried out in concentrated aqueous ammonia (33%) at 55° C. for 5 hours in a sealed tube. Fluorophores were attached to internal residues in the probe sequence using Fluorescein dT (Glen Research, Sterling, Va.). The oligonucleotides of the invention may possess a 3′-phosphate component or other blocking agent to prevent Taq mediated extension when the probes and UC oligonucleotides are incorporated into real-time PCR assays.

Junction probe oligonucleotides were also synthesised using a two-phase method. The first phase is a high scale synthesis of the universal element containing the expensive oligonucleotide components (i.e. fluorophore-labelled bases, terminal cap modifications and PCR blockers). The second phase takes a fraction of the universal element for extension of the target specific oligonucleotide sequence.

Synthesis of the universal element was performed using an AKTA Oligopilot DNA synthesiser, with a 20 μmol synthesis cycle. 500 mg of Synbase CPG 500/110 s solid support (Link Technologies) was loaded into a metal screw-fit column, to yield 20 μmol of functionalised resin. The synthesis of the universal sequence proceeded using standard coupling times and monomer equivalents for unmodified bases (2.5 minutes, 4 equivalents), and for modified bases coupling time was increased (20 minutes). Synthesis cycles were performed using standard conditions, and upon completion of the oligonucleotide synthesis the column was washed with acetonitrile, dried and the solid support removed for division.

The solid support functionalised with the universal sequence was weighed directly into plastic screw-fit columns design for use with ABI 394 oligonucleotide synthesisers (Applied Biosystems). 1 mg of the solid support was used for 40 nmol synthesis cycles and had to be weighed out taking suitable anti-static precautionary measures to ensure accurate measurement of the solid support. Multiple columns, each containing 40 nmol of functionalised solid support could then undergo further oligonucleotide synthesis cycles using the ABI 394 synthesisers, using standard cycle conditions (1.5 seconds coupling time, 50 equivalents).

Once synthesis of the target specific sequence was complete the oligonucleotide was cleaved from the resin and deprotected using concentrated aqueous ammonia at room temperature for one hour, followed by five hours at 55 degrees Celsius in a sealed tube. Any impurities from synthesis/deprotection were removed and the oligonucleotide desalted using Illustra NAP-10 gel filtration columns (GE Healthcare) according to the manufacturer's instructions.

The quantity of oligonucleotide obtained from the synthesis was determined by dissolving an aliquot in a specific volume of water and measuring the UV absorbance at 260 nm. Concentration was calculated using the UV absorbance of the oligonucleotide and its extinction coefficient at 260 nm. The extinction coefficient of the oligonucleotide was calculated from the sum of the individual extinction coefficients of the unmodified and fluorescently labelled nucleosides of which it is composed.

Standard HyBeacon@ probes were purified by HPLC whereas junction probes were purified by gel filtration. Universal complement, target oligonucleotides and PCR primers were purified by desalting.

### Oligonucleotide Melting Curve Analysis

Melting curve analysis was performed in 1× SpeedSTAR buffer II (TaKaRa) using 75 nM of junction probe, 500 nM universal complement oligonucleotide and 1 μM of target oligonucleotide. Oligonucleotide melting curve analysis was also performed using GoTaq colourless buffer (Promega), 3 mM MgCl₂, 150 nM junction probe, 200 nM universal complement and 500 nM of synthetic target. Alternate concentrations of oligonucleotides were tested and are described in specific examples below. Melting curves were generated using a CFX96 Real-Time PCR detection system (Bio-Rad) by briefly denaturing (98° C. 30 seconds) and cooling (20° C. 30 seconds) samples prior to increasing temperature from 20° C. to 75° C. in 0.5° C. steps, monitoring fluorescence emission at each step. Melting peaks were constructed using the CFX software by plotting the negative derivative of fluorescence with respect to temperature (−dF/dT on the y-axis) against temperature (x-axis).

### Polymerase Chain Reaction

PCR volumes were typically 20 μl, generally comprising 2 μl of sample, 1× Phire PCR buffer (Fisher Scientific, UK), 1 μM excess primer, 0.222 μM reverse primer, 0.4 μl Phire Hotstart II DNA polymerase (Fisher Scientific, UK), 3 mM total MgCl₂, 30 ng/μl BSA (Roche Diagnostics), 1 mM dNTPs (GE Healthcare), 75 nM of junction probe and 500 nM of universal complement oligonucleotide. Alternate PCR formulations were tested and are described in specific examples below.

The performance of junction probes was compared with 150 nM of standard HyBeacon®.

Templates for PCR (and isothermal amplification) were either synthetic oligonucleotides cloned into pUC75 vectors (Genscript, Piscataway, N.J., USA) or extracted DNA samples of known sequence/genotype that were previously characterised using validated methods using standard HyBeacon® probes.

Homogeneous amplification and detection of targets was performed with a CFX96 Real-Time PCR detection system (Bio-Rad) where, following an initial denaturation reaction step (98° C. 1 minute), targets were amplified using 50 cycles comprising denaturation (98° C. 5 seconds) and annealing/extension (65° C. 10 seconds). Melting curve analysis was performed immediately after amplification, by briefly denaturing (98° C. 30 seconds) and cooling (20° C. 30 seconds) samples prior to increasing temperature from 20° C. to 70° C. in 0.5° C. steps.

Annealing curve analysis was performed as an alternative to melting curve analysis. Samples were denatured (98° C. 30 seconds) and rapidly cooled (75° C. 30 seconds). Annealing curve analysis was performed by cooling samples from 75° C. to 35° C. in 0.5° C. steps, measuring fluorescence emission at the end of each step. Annealing peaks were constructed using the CFX software by plotting the negative derivative of fluorescence with respect to temperature (−dF/dT on the y-axis) against temperature (x-axis).

In example 13 the loop-mediated isothermal amplification method is used and the methods are describes therein.

### Example 1

The junction probe HRH2-JP was used with the universal complement oligonucleotide HRH2-UC to detect and differentiate the HRH2-T1 and HRH2-T2 target oligonucleotides (Table 1) through melting curve analysis. For HRH2-JP, the sequence 5′(AAAGFTTGFCAG)3′ acts as the universal element and 5′(CCACCGCCTTAGAG)3′ is the target specific element. For HRH2-UC, 5′(CCATTCGCAGACCTCG)3′ is the target specific sequence and 5′(CTGACAAACTTT)3′ is the complementary universal element.

The universal element of the HRH2-JP probe is located at the 5′ end of the oligonucleotide. Synthesis of universal probes can be performed 5′-3′ to generate the expensive fluorescent universal element prior to splitting the resin for synthesis of multiple target specific sequences. 5′-3′ oligonucleotide synthesis is possible but far less commonly used than 3′-5′ synthesis. Both synthesis methods may be useful depending on the nature of the target sequence.

TABLE 1
Oligonucleotide and oligonucleotide target
sequences, where F and Y represent fluorescein
dT and 3′ Pyrene dT, respectively. The
position of a target polymorphism is indicated.

| OLIGO | SEQUENCE | SEQ ID: |
|---|---|---|
| HRH2-JP | AAAGFTTGFCAGCCACCGCCTTAGAGY | 1 |
| HRH2-UC | CCATTCGCAGACCTCGCTGACAAACTTT | 2 |
| HRH2-T1 | CTCTAAGGCGGTGGCGAGGTCTGCGAATGG | 3 |
| HRH2-T2 | CTCTAAGGCGATGGCGAGGTCTGCGAATGG | 4 |

In the presence of HRH2-T1 the junction probe generated melting peaks at 54.0° C. In the presence of the HRH2-T2, the target mismatch reduced melting temperature to 45.5° C. (FIG. 3A). Melting peaks were not generated in the absence of universal complement oligonucleotide. Formation of the tribrid structure is required for detection and identification of target sequence, where probe needs to be hybridised with both the universal complement oligo and target sequence.

The HRH2 targets are clearly detected and discriminated with the junction probe but the stability of the universal element is too high. In the absence of target sequence the junction probe generated large peaks at 35.0° C. (FIG. 3A) through direct interaction with the universal complement oligonucleotide. Shorter and less stable universal elements are required to reliably detect target sequences.

### Example 2

The junction probe CDK-JP was used with the universal complement oligonucleotide CDK-UC to detect and discriminate the CDK-T1 and CDK-T2 target oligonucleotides (Table 2). For CDK-JP, the sequence 5′(ATTFCAATTAF)3′ acts as the universal element and 5′(GTCCCAACACTCAG)3′ is the target specific sequence. For CDK-UC, 5′(CCATTCGCAGACCTCG)3′ is the target specific sequence and 5′(ATAATTGAAAT)3′ is the complementary universal element.

TABLE 2
Oligonucleotide and oligonucleotide target
sequences, where F and Y represent fluorescein
dT and 3′ Pyrene dT, respectively. The
position of a target polymorphism is indicated.

| OLIGO | SEQUENCE | SEQ ID: |
|---|---|---|
| CDK-JP | ATTFCAATTAFGTCCCAACACTCAGY | 5 |
| CDK-UC | CCATTCGCAGACCTCGATAATTGAAAT | 6 |
| CDK-T1 | CTGAGTGTTGGGACCGAGGTCTGCGAAT | 7 |
| | GG | |
| CDK-T2 | CTGAGTGTTGAGACCGAGGTCTGCGAAT | 8 |
| | GG | |

Melting curve analysis with the CDK-T1 and CDK-T2 targets generated small inverted melting peaks with Tₘs of 48.5° C. and 42.5° C., respectively (FIG. 3B). Melting peaks were not generated in the absence of target sequence or when the junction probe was used in the absence universal complement oligonucleotide.

The tribrid structure does form with CDK-JP, CDK-UC and target oligonucleotides but the A/T rich sequence of the universal element does not yield high quality melting peaks. Formation of the tribrid structure will create a hydrophobic pocket at the point of junction. Having a fluorophore-labelled base at or near the junction can cause melting peaks to become inverted, where probe emission is quenched upon target hybridisation.

### Example 3

The junction probe AIH7-JP was used with the universal complement oligonucleotide AIH7-UC to detect and discriminate the AIH7-T1 and AIH7-T2 target oligonucleotides (Table 3). For AIH7-JP, 5′(TCGCGTGTGAGGAGAGGCC)3′ is the target specific sequence and 5′(TAFTTGGFGCTATAGC)3′ is the universal element. For AIH7-UC, 5′(AGCACCAAATA)3′ is the complementary universal element and 5′(CGAGGTCTGCGAATGG)3′ is the target specific sequence.

TABLE 3
Oligonucleotide and oligonucleotide target
sequences, where F and P represent fluorescein
dT and 3′ phosphate, respectively. The
position of target polymorphisms are indicated.

| | | SEQ |
|---|---|---|
| OLIGO | SEQUENCE | ID: |
| AIH7-JP | TCGCGTGTGAGGAGAGGCCTAFTTGGFGCTATA | 9 |
| | GCP | |
| AIH7-UC | AGCACCAAATACGAGGTCTGCGAATGG | 10 |
| AIH7-T1 | CCATTCGCAGACCTCGGGCCTCTCCTCACACGC | 11 |
| | GA | |
| AIH7-T2 | CCATTCGCAGACCTCGGACCTCTCCTCACACAC | 12 |
| | GA | |

Melting curve analysis with the AIH7-T1 and AIH7-T2 targets generated high quality (positive) melting peaks with Tₘs of 59.0° C. and 54.0° C., respectively (FIG. 3C). Melting peaks were not generated in the absence of universal complement oligonucleotide demonstrating that the tribrid structure is required for target detection.

Melting peaks at 31° C. were generated only in the absence of target sequence. An interaction between the universal elements of AIH7-JP and AIH7-UC can occur in the absence of target but this does not compromise the sensitivity or reliability of target detection or discrimination. Melting curve analysis between ˜40-75° C. would reliably detect matched and mismatched target sequences and would prevent interaction of the probe and universal complement in the absence of target. Alternatively, the universal element specific peak could be used to indicate the absence of target. Furthermore, simultaneous detection of target and universal element peaks could indicate the presence of low copy number targets.

### Example 4

The junction probe CXC-JP was used with the universal complement oligonucleotide CXC-UC to detect and discriminate the CXC-T1 and CXC-T2 target oligonucleotides (Table 4). For CXC-JP, the sequence 5′(GTGGFAGGATF)3′ acts as the universal element and 5′(GAGCGAGTCAGG)3′ is the target specific sequence. For CXC-UC, 5′(CCATTCGCAGACCTCG)3′ is the target specific sequence and 5′(AATCCTACCAC)3′ is the complementary universal element.

Melting curve analysis with the CXC-T1 and CXC-T2 targets generated high quality (positive) melting peaks with Tₘs of 54.0° C. and 43.0° C., respectively (FIG. 3D). Melting peaks were not generated in the absence of universal complement oligonucleotide demonstrating that the tribrid structure is required for target detection.

Melting peaks at 22° C. were generated only in the absence of target sequence. An interaction between the universal elements of CXC-JP and CXC-UC can occur in the absence of target but performing melting curve analysis between 35° C. and 75° C. will not permit this structure to form.

The target oligonucleotide CXC-T3 has an additional C nucleotide at the position of the tribrid junction (formed by hybridisation of probe, universal complement and target sequence). The additional non-hybridised target nucleotide reduces the probe Tₘ by approximately 1° C. but does not improve or reduce the height/quality of melting peaks.

One of the CXC-JP fluorophores is located at the junction formed with the universal complement oligonucleotide and target sequence. The sequence of the universal element yields fluorescence enhancement and positive melting peaks upon target hybridisation.

TABLE 4
Oligonucleotide and oligonucleotide target
sequences, where F and Y represent fluorescein
dT and 3′ Pyrene dT, respectively. The
position of a target polymorphism is indicated.

| | | SEQ |
|---|---|---|
| OLIGO | SEQUENCE | ID: |
| CXC-JP | GTGGFAGGATFGAGCGAGTCAGGY | 13 |
| CXC-UC | CCATTCGCAGACCTCGAATCCTACCAC | 14 |
| CXC-T1 | CCTGACTCGCTCCGAGGTCTGCGAATGG | 15 |
| CXC-T2 | CCTGACTCACTCCGAGGTCTGCGAATGG | 16 |
| CXC-T3 | CCTGACTCGCTCCCGAGGTCTGCGAATGG | 17 |

### Example 5

The oligonucleotides described in the examples above were generated using standard 3′-5′ synthesis methods. Subsequent examples used two-phase methods to synthesise junction probe oligonucleotides. Junction probes were synthesised 3′-5′ to locate the expensive fluorescent universal element at the 3′ end of the oligonucleotide. Probes were synthesised in two steps, the first step being high scale synthesis of the universal element. The second step involves splitting the synthesis resin containing the universal element and then extending the oligonucleotides with different target specific sequences. The synthesis method considerably reduces cost and the universal element simplifies probe design.

The junction probes G1-JP, P2-JP, CDK2-JP and CH-JP (Table 5) all use the universal element sequence 5′(GFTAGGAFGGTG)3′. The universal complement oligonucleotides G1-UC, P2-UC, CDK2-UC and CH-UC all have the complementary universal element sequence 5′(CCATCCTAAC)3′. The target specific sequences of junction probes and universal complement oligonucleotides are detailed in Table 5.

The junction probe G1-JP was used with the universal complement oligonucleotide G1-UC to detect and discriminate the polymorphic G1-T1 and G1-T2 target oligonucleotides (Table 5). Melting curve analysis with the G1-T1 and G1-T2 targets generated high quality (inverted) melting peaks with Tₘs of 51.0° C. and 40.5° C., respectively (FIG. 4A). Melting peaks were not generated in the absence of target or universal complement oligonucleotide. Target detection and identification requires hybridisation of all three sequence elements of the tribrid structure (i.e. junction probe, universal complement and target sequence).

The junction probe P2-JP was used with the universal complement oligonucleotide P2-UC to detect and discriminate the polymorphic P2-T1 and P2-T2 target oligonucleotides. Melting curve analysis with the P2-T1 and P2-T2 targets generated high quality (inverted) melting peaks with Tₘs of 49.5° C. and 36.0° C., respectively (FIG. 4B). Melting peaks were not generated in the absence of target sequence or universal complement oligonucleotide.

The junction probe CDK2-JP was used with the universal complement oligonucleotide CDK2-UC to detect and discriminate the polymorphic CDK2-T1 and CDK2-T2 target oligonucleotides. Melting curve analysis with the CDK2-T1 and CDK2-T2 targets generated inverted melting peaks with Tₘs of 45.0° C. and 35.5° C., respectively (FIG. 4C). Melting peaks were not generated in the absence of target sequence or UC oligonucleotide.

The junction probe CH-JP was used with the universal complement oligonucleotide CH-UC to detect and discriminate the polymorphic CH-T1 and CH-T2 target oligonucleotides. Melting curve analysis with the CDK2-T1 and CH-T2 targets generated high quality (inverted) melting peaks with Tₘs of 50.0° C. and 40.0° C., respectively (FIG. 4D). Melting peaks were not generated in the absence of target sequence or UC oligonucleotide.

TABLE 5
Oligonucleotide and oligonucleotide target
sequences, where F, P and 7 represent
fluorescein dT, 3′ phosphate and
3′ amino C7, respectively. The
position of target polymorphisms are indicated.

| | | SEQ |
|---|---|---|
| OLIGO | SEQUENCE | ID: |
| G1-JP | CCATAACAGCAAGGFTAGGAFGGTGP | 18 |
| G1-UC | CCATCCTAACAGCTTCGAAAAAGTTTTCTTTAC7 | 19 |
| G1-T1 | GTAAAGAAAACTTTTTCGAAGCTCTTGCTGTTATGG | 20 |
| G1-T2 | GTAAAGAAAACTTTTTCGAAGCTCTTGCTATTATGG | 21 |
| P2-JP | GGGGATCGATTGFTAGGAFGGTGP | 22 |
| P2-UC | CCATCCTAACGAAACTCTTTTTGTAGAGTTTG7 | 23 |
| P2-T1 | CAAACTCTACAAAAAGAGTTTCAATCGATCCCC | 24 |
| P2-T2 | CAAACTCTACAAAAAGAGTTTCAATCAATCCCC | 25 |
| CDK2-JP | TTATGTCCCAACAGFTAGGAFGGTG | 26 |
| CDK2-UC | CCATCCTAACCTCAGCTTAGATTTGACCCAC7 | 27 |
| CDK2-T1 | GTGGGTCAAATCTAAGCTGAGTGTTGGGACATAA | 28 |
| CDK-T2 | GTGGGTCAAATCTAAGCTGAGTGTTGAGACATAA | 29 |
| CH-JP | GTAATCAAGCCTGGFTAGGAFGGTGP | 30 |
| CH-UC | CCATCCTAACCAAATGTATACCAAGAAATAAAAATG7 | 31 |
| CH-T1 | CATTTTTATTTCTTGGTATACATTTGCAGGCTTGATT | 32 |
| | AC | |
| CH-T2 | CATTTTTATTTCTTGGTATACATTTGCAGGCTTAATT | 33 |
| | AC | |

The universal element sequence used in the G1-JP, P2-JP, CDK2-JP and CH-JP probes yields inverted melting peaks with matched and mismatched target oligonucleotides. Formation of the tribrid structure causes fluorescence quenching, possibly due to the proximity of a fluorophore-labelled base to the hydrophobic pocket formed at the junction of the three DNA sequences. The DNA sequence flanking the fluorophore-labelled bases (and the sequence in the target) may also influence whether emission is enhanced or quenched on target hybridisation. Flanking and target sequences can affect the magnitude of fluorescence enhancement with standard HyBeacon® probes upon hybridisation, but fluorescence quenching is not typically observed.

### Example 6

The junction probes G1-JPv2 and P2-JPv2 (Table 6) used the universal element sequence 5′(TAGFTAGGAFGGTG)3′ and the corresponding universal complement oligonucleotides G1-UCv2 and P2-UCv2 had the universal element sequence 5′(CATCCTAACTA)3′. The TA nucleotides at the 5′ end of the probe universal element were added (to the probes of example 5) to increase the distance between the fluorophore-labelled bases and the hydrophobic pocket formed at the tribrid junction.

The junction probe G1-JPv2 was used with the universal complement oligonucleotide G1-UCv2 to detect and discriminate the polymorphic G1-T1 and G1-T2 target oligonucleotides (Table 5). Melting curve analysis with the G1-T1 and G1-T2 targets generated high quality (positive) melting peaks with Tₘs of 48.5° C. and 38.0° C., respectively (FIG. 5A). Detectable melting peaks were not generated in the absence of target sequence. Any interaction between the universal elements of junction probe and universal complement oligonucleotides would not occur above 30° C.

Melting curve analysis was also performed with 150 nM of the G1-JPv2 probe using the linear target oligonucleotides G1-JPv2RC and G1-JPv2RC2 (Table 6), which hybridise to form standard duplexes rather than tribrid structures. The melting temperatures generated with the matched and mismatched linear targets were 58.5° C. and 50.0° C., respectively. This demonstrates that probe stability in the tribrid is considerably lower than when hybridised to a linear target. The destabilising effect of the tribrid junction needs to be considered when designing universal probes and complement oligonucleotides (see example 20).

TABLE 6
Oligonucleotide and oligonucleotide target
sequences, where F, P and 7 represent
fluorescein dT, 3′ phosphate and
3′ amino C7, respectively. The
position of a target polymorphism is indicated.

| OLIGO | SEQUENCE | SEQ ID: |
|---|---|---|
| G1-JPv2 | CCATAACAGCAAGTAGFTAGGAFGGTGP | 34 |
| G1-UCv2 | CATCCTAACTAAGCTTCGAAAAAGTTTTC | 35 |
| | TTTAC7 | |
| G1-JPv2RC | CATCCTAACTACTTGCTGTTATGG | 36 |
| G1-JPv2RC2 | CATCCTAACTACTTGCTATTATGG | 37 |
| P2-JPv2 | GGGGATCGATTTAGFTAGGAFGGTGP | 38 |
| P2-UCv2 | CATCCTAACTAGAAACTCTTTTTGTAGAGT | 39 |
| | TTGP | |

The junction probe P2-JPv2 was used with the universal complement oligonucleotide P2-UCv2 to detect and discriminate the polymorphic P2-T1 and P2-T2 target oligonucleotides (Table 5). Melting curve analysis with the G1-T1 and G1-T2 targets generated high quality (positive) melting peaks with Tₘs of 45.5° C. and 32.5° C., respectively (FIG. 5B). Detectable melting peaks were not generated in the absence of target sequence.

Moving the fluorophore-labelled bases further away from the hydrophobic junction has resulted in fluorescence enhancement upon probe hybridisation. The additional TA nucleotides in the universal element sequence have changed the inverted melting peaks observed with G1-JP and P2-JP into positive peaks with G1-JPv2 and P2-JPv2 probes.

Annealing curve analysis was performed as an alternative to melting curve analysis, where fluorescence is acquired as samples cool and the tribrid structure is formed between junction probe, universal complement and target sequence.

Melting and annealing curve analysis using 150 nM G1-JPv2, 150 nM G1-UCv2 and 150 nM target oligonucleotide are compared in FIG. 5C and FIG. 5D, respectively.

### Example 7

The functionality of the G1-JPv2 and P2-JPv2 probes was tested with PCR amplified targets sequences. Target sequences for the G1-JPv2 probe were amplified using 1 μM G1-LF and 222 nM G1-LR primers (Table 7). Target sequences for the P2-JPv2 probe were amplified with 1 μM P2-LF and 500 nM P2-LR primers. Targets were amplified from synthetic DNA sequences constructed using gene synthesis (GenScript, Hong Kong). Synthetic constructs comprised primer, junction probe and universal complement regions which were cloned into pUC57 vectors and sequenced for quality control.

The performance and sensitivity of the junction probe PCRs was compared with the standard HyBeacon® probes G1-L and P2-L (Table 7). The junction probes generated high quality melting peaks with PCR amplified targets sequences (FIG. 6). Peak heights were reduced compared with standard HyBeacon® probes but the same level of sensitivity was achieved (approximately 18 target copies).

TABLE 7
PCR primer and standard HyBeacon ® sequences,
where F and Y represent fluorescein dT and
3′ Pyrene dT, respectively.

| OLIGO | SEQUENCE | SEQ ID: |
|---|---|---|
| G1-L | CTTCACGFTCCAFAACAGCAAGAGY | 40 |
| G1-LF | CGGAGTTCCTAGAGGATTCCTAGACG | 41 |
| G1-LR | TACCGCATGTTTCAAAACACTGAGA | 42 |
| P2-L | GGATCGATFGAAACFCTTTTTGTAGAY | 43 |
| P2-LF | CATATTACGAGCTTTTTATAAACCTCCCCA | 44 |
| | AC | |
| P2-LR | TCGCCGATGAGTTCGACATTCC | 45 |

### Example 8

The effect of target sequence polymorphism was investigated using the G1-JPv2 probe. Oligonucleotides were synthesised with mismatched positions located within the target sequences of junction probe and universal complement. The oligonucleotide G1-T1 (Table 5) is fully complementary to the G1-JPv2 probe and G1-UCv2 universal complement. The oligonucleotides G1-T2 (Table 5), G1-T3, G1-T4 and G1-T5 (Table 8) have mismatches in the probe target. The oligonucleotides G1-T6 and G1-T7 (Table 8) have mismatches in the target of the universal complement.

A mismatch in the middle of the probe target (G1-T2) causes a 10° C. reduction of melting peak Tₘ (Table 9), whereas a mismatch located towards the 5′ end of the probe (G1-T3) only reduces the Tₘ by 4.5° C. The least destabilising probe mismatch was located at the junction where probe and universal complement oligonucleotides meet on the target sequence (G1-T4), where peak Tₘ was reduced by only 2° C. relative to G1-T1 (Table 9).

TABLE 8
Universal complement and target oligonucleotide
sequences with polymorphic positions indicated.

| OLIGO | SEQUENCE | SEQ ID: |
|---|---|---|
| G1-T3 | GTAAAGAAAACTTTTTCGAAGCTCTTGCTGTTA | 46 |
| | TAG | |
| G1-T4 | GTAAAGAAAACTTTTTCGAAGCTATTGCTGTTA | 47 |
| | TAG | |
| G1-T5 | GTAAAGAAAACTTTTTCGAAGCTATTGCTATTAT | 48 |
| | GG | |
| G1-T6 | GTAAAGAAAACTTTTTCAAAGCTCTTGCTGTTAT | 49 |
| | GG | |
| G1-T7 | GTAAATAAAACTTTTTCAAAGCTCTTGCTGTTAT | 50 |
| | GG | |
| G1-UCv3 | CATCCTAACTAAGCTTCGAAAAAGTTTT | 51 |
| G1-UCv4 | CATCCTAACTAAGCTTCGAAAAAG | 52 |

The magnitude of melting peak Tₘ reduction is determined by the type and location of the probe target mismatch. Probes are typically designed to detect known polymorphisms and will reliably generate melting peaks with Tₘs that vary by less than ±1° C. Additional sites of polymorphism can be neutralised using universal bases such as 5-nitroindole or can be detected through the generation of unexpected melting peak Tₘs. Additional polymorphisms can also be detected when sequence mismatches are located within the universal complement target.

The long G1-UCv2 oligonucleotide is tolerant to a single destabilising C/A mismatch within the universal complement target sequence (G1-T6), but two mismatches within the universal complement (G1-T7) cause the melting peak Tₘ to be reduced by 2.5° C. The shorter universal complement oligonucleotides G1-UCv3 and G1-UCv4 (Table 8) are destabilised more by target mismatches and melting peak Tₘs are reduced further than with G1-UCv2 (Table 9). Melting peak Tₘ can be determined by the dissociation temperature of universal complement if the stability of the universal complement oligonucleotide is less than the stability of the junction probe. Early dissociation of the universal complement oligonucleotide could cause miscalls in the presence of unknown polymorphisms. For example, the G1-JPv2 probe generates similar melting peak Tₘs with G1-T2 and G1-T6 target mismatches using the G1-UCv4 universal complement.

TABLE 9
Melting peak Tₘs generated with the G1-JPv2 probe

| OLIGO | MISMATCH | G1-UCv2 | G1-UCv3 | G1-UCv4 |
|---|---|---|---|---|
| G1-T1 | | 48.0° C. | 48.0° C. | 46.5° C. |
| G1-T2 | C/A | 38.0° C. | 38.0° C. | 38.0° C. |
| G1-T3 | C/A | 43.5° C. | 43.0° C. | 52.5° C. |
| G1-T4 | C/A | 46.0° C. | 46.0° C. | 45.0° C. |
| G1-T5 | 2x C/A | 35.5° C. | 35.5° C. | 35.0° C. |
| G1-T6 | C/A | 48.0° C. | 44.5° C. | 39.0° C. |
| G1-T7 | C/T & C/A | 45.5° C. | 44.5° C. | 38.5° C. |

### Example 9

The junction probe CH-JPv2-UE1 (Table 10) used the universal element sequence 5′(TAGFTAGGAFGGTG)3′. The TA nucleotides at the 5′ end of the universal element were added to the CH-JP probe (of example 6) to increase the distance between the fluorophore-labelled bases and the hydrophobic pocket formed at the tribrid junction. The universal complement oligonucleotide CH-UCv2-UE1 had the universal element sequence 5′(CACCATCCTAACTA)3′. The CH-JP probe generated inverted melting peaks (FIG. 4D) but the additional universal bases yielded high quality positive melting peaks with the CH-T1 target sequence (FIG. 7)

As observed with the G1-JPv2 and P2-JPv2 probes, moving the fluorophore-labelled bases further away from the hydrophobic junction has resulted in fluorescence enhancement upon probe hybridisation. The additional TA nucleotides in the universal element sequence have changed the inverted melting peaks observed with CH-JP into positive peaks with CH-JPv2-UE1.

TABLE 10
Oligonucleotide and oligonucleotide target
sequences, where F, p and S represent
fluorescein dT, 3′ phosphate and
SIMA dT, respectively. The position of a
target polymorphism is indicated.

| | | SEQ |
|---|---|---|
| OLIGO | SEQUENCE | ID: |
| CH-JPv2-UE1 | GTAATCAAGCCTGTAGFTAGGAFGGTGp | 53 |
| CH-JPv2-UE2 | GTAATCAAGCCTGTATFGGAGAFGTGCp | 54 |
| CH-UCv2-UE1 | CACCATCCTAACTACAAATGTATACCAAGp | 55 |
| CH-UCv2-UE2 | GCACATCTCCAATACAAATGTATACCAAGp | 56 |
| CH-JPv2-SIMA | GTAATCAAGCCTGTAGSTAGGASGGTGp | 57 |

Additional junction probe and universal complement oligonucleotides were designed to detect the CH-T1 target sequence (Table 5). The junction probe CH-JPv2-UE2 used the universal element sequence 5′(TATFGGAGAFGTGC)3′ and the universal complement oligonucleotide CH-UCv2-UE2 had the universal element sequence 5′(GCACATCTCCAATA)3′. Melting curve analysis with the CH-JPv2-UE2, CH-UCv2-UE2 and CH-T1 oligonucleotides generated high quality positive melting peaks at approximately 50° C. (FIG. 7).

Both the CH-JPv2-UE1 and CH-JPv2-UE2 probes generate positive melting peaks using different universal element sequences, where fluorophore-labelled bases are separated from the tribrid junction by three and nine nucleotides.

The target specific sequences of CH-JPv2-UE1 and CH-JPv2-UE2 probes are identical (Table 10). The target specific sequences of CH-UCv2-UE1 and CH-UCv2-UE2 oligonucleotides are also identical. Melting peaks are not generated when the CH-JPv2-UE1 probe is used with the CH-UCv2-UE2 universal complement oligonucleotide. Peaks are also not generated when the CH-JPv2-UE2 probe is used with the CH-UCv2-UE1 universal complement oligonucleotide (FIG. 7). This demonstrates that the different universal element sequences do not cross-react and that probe hybridisation to both the target sequence and universal complement oligonucleotide is required for target detection and sequence identification.

Since universal element sequences can be designed to prevent interaction of probe and universal complement oligonucleotides in the absence of target, the same universal element sequence could be used for multiple target sequences. Melting peaks will not be generated unless the target specific elements of probe and complement oligonucleotides are adjacent on the target sequence.

Multiplex analysis may be performed with universal HyBeacon®s using the same or different universal element sequences.

### Example 10

The CH-JPv2-SIMA probe (Table 10) was labelled with SIMA dT rather than fluorescein dT. Melting curve analysis with the CH-JPv2-SIMA, CH-UCv2-UE1 and CH-T1 oligonucleotides yielded high quality melting peaks (FIG. 8A).

SIMA is a fluorescein based dye with absorption and emission spectra virtually identical to HEX (535 nm Ex, 556 nm Em). Fluorescein and SIMA dyes may be used simultaneously for multiplex analysis.

The combination of CH-JPv2-SIMA probe and CH-UCv2-UE1 universal complement was used in a multiplex with JP-22 and UC-22 oligonucleotides (Table 11). The fluorescein-labelled JP-22 probe used the universal element sequence 5′(GTAFTATGAFATTG)3′ and the universal complement had the universal element 5′(CAATATCATAATAC)3′. When the JP-22 and CH-JPv2-SIMA probes were used together, the T-22 and CH-T3 (Table 11) target sequences were detected simultaneously using FAM and SIMA dye channels of real-time PCR instrumentation (FIG. 8B). When only one target sequence is present only one of the instrument dye channels yields the appropriate melting peak. Detection of the T-22 and CH-T1 target sequences yields melting peaks with Tₘs of approximately 43° C. and 59° C. using the JP-22 and CH-JPv2-SIMA probes. This separation of peaks could allow simultaneous detection of both target sequences if probes were both labelled with the same fluorescent dye.

The CH-JPv2-UE1 probe (Table 10) was used to detect plasmid target sequence amplified from extracted Chlamydia trachomatis DNA. Target amplification was performed using PCR and the primers CH-F and CH-R (Table 11). Target detection was achieved using a tribrid structure formed with the CH-JPv2-UE1 junction probe and CH-UC4v2 universal complement oligonucleotide (Table 11).

The S1-JP probe was used to detect genomic target sequence amplified from extracted Chlamydia trachomatis DNA. Target amplification was performed using PCR and the primers S1-F and S1-R (Table 11). Target detection was achieved using a tribrid structure formed with the S1-JP probe and S1-UC universal complement oligonucleotide (Table 11).

The Chlamydia trachomatis plasmid and genomic target sequences were detected simultaneously in a multiplex PCR test using the fluorescein-labelled CH-JPv2-UE1 and SIMA-labelled S1-JP probes. PCR reaction volumes were 20 μl, comprising 2 μl sample, 1× AptaTaq mastermix (Roche Applied Science), 1 mM dNTPs, 30 ng/μl BSA, 1 μM S1-F primer, 222 nM S1-R primer, 222 nM CH-F primer, 1 μM CH-R primer, 75 nM S1-JP probe, 1 μM S1-UC oligo, 75 nM CH-JPv2-UE1 probe and 500 nM CH-UC4v2 oligo. PCR amplification was performed using a CFX96 thermal protocol, where following an initial denaturation step (98° C. 1 minute), targets were amplified using 50 cycles comprising denaturation (98° C. 5 seconds) and annealing/extension (65° C. 10 seconds). Melting curve analysis was performed immediately after amplification by briefly denaturing (98° C. 30 seconds) and cooling (30° C. 30 seconds) samples prior to increasing temperature from 30° C. to 65° C. in 0.5° C. steps. Fluorescence acquisition was performed in fluorescein and SIMA dye channels for multiplex detection of amplified targets.

Multiplex analysis clearly detects the plasmid and genomic targets of Chlamydia trachomatis using a dilution series of extracted DNA containing between 1.8×10⁵ and 1.8 genomic copies. The fluorescein-labelled CH-JPv2-UE1 and SIMA-labelled S1-JP probes generate melting peaks with Tms of approximately 47° C. (FIG. 8C) and 49° C. (FIG. 8D), respectively. Peaks were not generated in the absence of target sequence.

TABLE 11
Oligonucleotide and oligonucleotide target
sequence, p, 7 and S represent 3′ phosphate,
3′ amino C7 and SIMA dT, respectively.

| OLIGO | SEQUENCE | SEQ ID: |
|---|---|---|
| JP-22 | CTTTGTAATCAAGCCTGTAFTATGAFATTG | 58 |
| UC-22 | CAATATCATAATACGCAAATGTATACCAAGAAA | 59 |
| | TAA | |
| T-22 | GGATGTACAACAATATCTGTTAGTCACCTTTGG | 60 |
| | GTC | |
| CH-UC4v2 | CATCCTAACTACAAATGTATACCAAGAAATAAA | 61 |
| | AATGp | |
| CH-F | GGGTTCGTTGTAGAGCCATGTCCTATCTTG | 62 |
| CH-R | CGCAGCTGCTGTAATCACCCAGTCGATAAA | 63 |
| S1-JP | CCACCGTATGGCTAAGGGTASTATGASATTG | 64 |
| S1-UC | CAATATCATAATACAGATTCGCTTTAAGAAGGC | 65 |
| | T7 | |
| S1-F | GGGCTTTGTTCACCTCTTTCAACG | 66 |
| S1-R | CCTCTGCGTAACTTCTCTTCCG | 67 |

Three different universal element sequences are presented here in examples, using probes constructed using the novel two-phase synthesis protocol. The probe universal elements 5′(TAGFTAGGAFGGTG)3′, 5′(TATFGGAGAFGTGC)3′ and 5′(GTAFTATGAFATTG)3′ all yield positive melting peaks and are suitable for multiplex analysis. The three universal elements have the fluorescent-labelled bases positioned three and nine nucleotides away from the hydrophobic pocket formed at the tribrid junction.

### Example 11

Additional target examples are provided below to demonstrate the universal nature of one of the junction probe designs. Tables 12 & 13 present the sequences of junction probes JP-1 to JP-8 and the corresponding universal complement oligonucleotides UC-1 to UC-8. In each case the probes and UC oligonucleotides used the universal element sequences 5′(GTAFTATGAFATTG)3′ and 5′(CAATATCATAATAC)3′, respectively. Melting curve analysis was performed with oligonucleotide target sequences (Tables 12 & 13). The junction probe, universal complement oligonucleotides and target oligonucleotides were all used at 150 nM.

The combination of JP-1 and UC-1 clearly detects and discriminates the T-1.1 and T-1.2 targets differing by a single T>C polymorphism (FIG. 9A).

The combination of JP-2 and UC-2 allows simultaneous analysis of G>C and C>T polymorphisms within the T-2.1, T-2.2 and T-2.3 target sequences, with melting curve analysis generating three clear melting peaks (FIG. 9B).

The combination of JP-3 and UC-3 oligonucleotides clearly detects and discriminates the T-3.1 and T-3.2 targets which differ by a single T>G polymorphism (FIG. 9C).

The combination of JP-4 and UC-4 oligonucleotides clearly detects and discriminates the T-4.1 and T-4.2 targets which differ by a single G>A polymorphism (FIG. 9D).

The combination of JP-5 and UC-5 oligonucleotides clearly detects and discriminates the T-5.1 and T-5.2 targets which differ in length by a single nucleotide, possessing and insertion/deletion polymorphism of an A nucleotide (FIG. 10A).

The combination of JP-6 and UC-6 oligonucleotides clearly detects and discriminates the T-6.1 and T-6.2 targets which differ in length by three nucleotides, possessing and insertion/deletion polymorphism of TCT nucleotides (FIG. 10B).

The combination of JP-7 and UC-7 oligonucleotides generates a small peak Tₘ difference with T-7.1 and T-7.2 targets which differ by a single A>G polymorphism (FIG. 10C). The small ΔTₘ between peaks is due to the proximity of the polymorphism to the hydrophobic pocket formed at the junction of junction probe, universal complement and target sequences.

The combination of JP-8 and UC-8 oligonucleotides clearly detects and discriminates the T-8.1 and T-8.2 targets which differ by a single G>A polymorphism (FIG. 10D).

TABLE 12
Oligonucleotide and oligonucleotide target
sequence, where F and p represents fluorescein
dT and 3′ propanol, respectively. The
positions of target polymorphisms are
indicated.

| OLIGO | SEQUENCE | SEQ ID: |
|---|---|---|
| JP-1 | GGATCTGGATGATGGTAFTATGAFATTG | 68 |
| UC-1 | CAATATCATAATACGGCACAGGCGGGCp | 69 |
| T-1.1 | GCCCGCCTGTGCCCATCATCCAGATCC | 70 |
| T-1.2 | GCCCGCCTGTGCCCATCACCCAGATCC | 71 |
| JP-2 | CGCTTCTCCGTCTCCGTAFTATGAFATTG | 72 |
| UC-2 | CAATATCATAATACACCTTGCGCAACTTGGGp | 73 |
| T-2.1 | CCCAAGTTGCGCAAGGTGGAGACGGAGAAGCG | 74 |
| T-2.2 | CCCAAGTTGCGCAAGGTGGAGATGGAGAAGCG | 75 |
| T-2.3 | CCCAAGTTGCGCAAGGTGGACACGGAGAAGCG | 76 |
| JP-3 | TACATCCGGATGGTAFTATGAFATTG | 77 |
| UC-3 | CAATATCATAATACTGCAGCGTGAGCCCAp | 78 |
| T-3.1 | TGGGCTCACGCTGCACATCCGGATGTA | 79 |
| T-3.2 | TGGGCTCACGCTGCACATCCGGAGGTA | 80 |
| JP-4 | GCGTCCTGGGGTAFTATGAFATTG | 81 |
| UC-4 | CAATATCATAATACGGTGGGAGATGCGGGp | 82 |
| T-4.1 | CCCGCATCTCCCACCCCCAGGACGC | 83 |
| T-4.1 | CCCGCATCTCCCACCCCCAGAACGC | 84 |

TABLE 13
Oligonucleotide and oligonucleotide target
sequence, where F and p represents fluorescein
dT and 3′ propanol, respectively.
The positions of target polymorphisms
are indicated.

| OLIGO | SEQUENCE | SEQ ID: |
|---|---|---|
| JP-5 | TCATCCTGTGCTCGTAFTATGAFATTG | 85 |
| UC-5 | CAATATCATAATACAGTTAGCAGCTCATCCAGCp | 86 |
| T-5.1 | GCTGGATGAGCTGCTAACTGAGCACAGGATGA | 87 |
| T-5.2 | GCTGGATGAGCTGCTAACTGAGCACGGATGA | 88 |
| JP-6 | GATGGAGAAGGTGAGTAFTATGAFATTG | 89 |
| UC-6 | CAATATCATAATACGAGTGGCTGCCACGGTp | 90 |
| T-6.1 | ACCGTGGCAGCCACTCTCACCTTCTCCATC | 91 |
| T-6.2 | ACCGTGGCAGCCACTCTCACCTCCATC | 92 |
| JP-7 | ATGAGAACCTGTGCGTAFTATGAFATTG | 93 |
| UC-7 | CAATATCATAATACTAGTGGTGGCTGACCTGp | 94 |
| T-7.1 | CAGGTCAGCCACCACTAGCACAGGTTCTCAT | 95 |
| T-7.2 | CAGGTCAGCCACCACTAGCGCAGGTTCTCAT | 96 |
| JP-8 | CCCTTCCTCCCGTAFTATGAFATTG | 97 |
| UC-8 | CAATATCATAATACTCGGCCCCTGCACp | 98 |
| T-8.1 | GTGCAGGGGCCGAGGGAGGAAGGG | 99 |
| T-8.2 | GTGCAGGGGCCGAGGGAGAAAGGG | 100 |

The junction probes of these examples all generate positive melting peaks with each of the target sequences.

### Example 12

The tribrid structure removes the strict requirement for a 3′ PCR blocker on the junction probe since target is not available for probe extension on the target DNA sequence. Junction probes without 3′ blockers have been tested successfully in PCR tests. The CH-JPv2-UE1 junction probe (Table 10) was compared with the CH-JPv2-UE1np oligonucleotide of the same sequence lacking the 3′ phosphate modification (Table 14). Junction probes were used along with the CH-UCv2-UE1 universal complement oligonucleotide for target detection. Target sequences were amplified with the primers CH-F and CH-R (Table 11) using synthetic DNA template constructed using gene synthesis (GenScript, Hong Kong). Synthetic constructs comprised primer, junction probe and universal complement regions which were cloned into pUC57 vectors and sequenced for quality control. Both the CH-JPv2-UE1 and CH-JPv2-UE1np junction probes generated a single melting peak at approximately 48° C. with amplified target sequence (FIG. 11A). Additional higher Tₘ melting peaks arising from PCR extension from the probe oligonucleotide were not observed.

TABLE 14
Oligonucleotide sequence, where F represents
fluorescein dT.

| OLIGO | SEQUENCE | SEQ ID: |
|---|---|---|
| CH-JPv2- | GTAATCAAGCCTGTAGFTAGGAFGGTG | 101 |
| UE1np | | |

The preferred embodiment of the invention is to have junction probes and universal complement oligonucleotides capped at the 3′ ends with 3′ phosphate, 3′ propanol, 3′ amino C7, 3′ pyrene or other 3′ modifications to prevent extension in the event of spurious target hybridisation or oligonucleotide dimer formation.

Target amplification using the S1-F and S1-R PCR primers and detection with the S1-JP and S1-UC oligonucleotides (Table 11) specifically detects the target amplicon when 75 nM junction probe and 1 μM universal complement are used, generating a melting peak at approximately 52° C. (FIG. 11B). A melting peak at approximately 72° C. is generated in no template controls (NTCs) and when higher probe concentrations are used. The 72° C. melting peak arises from PCR and is not generated in the absence of polymerase enzyme. A self dimer between S1-JP oligonucleotides causes spurious product amplification which is detected without the requirement for tribrid formation (FIG. 11B).

### Example 13

The junction probe S1-JP and universal complement oligonucleotide S1-UC (Table 11) were used to detect target sequence amplified isothermally. The oligonucleotides detailed in Table 15 were used to amplify the S1 target sequence using Loop-Mediated Isothermal Amplification (Notomi et al., 2000). 800 nM of the S1-FIP and S1-BIP primers were used with 200 nM of the S1-F3, S1-B3 and S1-LB oligonucleotides. Reactions also contained 150 nM of the S1-JP and 500 nM of the S1-UC oligonucleotides. Targets were amplified using an isothermal mastermix, without intercalating dye, available from OptiGene (Horsham, UK). After 30-60 minutes of isothermal amplification at 65° C., targets were detected by melting curve analysis (FIG. 12). Melting peaks were generated only in the presence of target sequence.

TABLE 15
Oligonucleotide sequences used for
Loop-Mediated Isothermal Amplification.

| OLIGO | SEQUENCE | SEQ ID: |
|---|---|---|
| S1-FIP | TTGCAGCTCCTTCTCTTGTTCCGTGGAGCAA | 102 |
| | GCCTTCTTAA | |
| S1-BIP | GCCTATTGTAGTGCGGAAGAGAATCGGTCT | 103 |
| | AAGCTTCCTAATTC | |
| S1-F3 | GTTCACCTCTTTCAACGGT | 104 |
| S1-B3 | TCGTGTTCTCCAAGAGTCT | 105 |
| S1-LB | AGGCTGTGGAGCAGTTAATC | 106 |

### Example 14

The junction probe G1-JPrc was used with the universal complement oligonucleotide G1-UCrc (Table 16) to detect and discriminate the G1-T1 and G1-T2 target sequences (Table 5). The G1-JPrc and G1-UCrc use the universal element sequences 5′(CCAFCCFAAC)3′ and 5′(GTTAGGATGGTG)3′, respectively. Formation of a tribrid structure between G1-JPrc, G1-UCrc and target sequences results in inverted melting peaks at 37° C. and 48° C. with G1-T2 and G1-T1 targets, respectively (FIG. 13A). In this example the length of the universal element in the complement oligonucleotide is greater than that used in the junction probe. High quality melting peaks are generated when fluorophore-labelled bases are separated by as little as two nucleotides.

The junction probe G1-JPrc has the same nucleotide sequence as the G1-UC oligonucleotide described in example 5. The universal complement oligonucleotide G1-UCrc also has the same nucleotide sequence as the G1-JP probe (Table 5). As such, the G1-JP and G1-JPrc junction probes have complementary universal elements and can be used together to detect target sequence. High quality inverted melting peaks are generated with G1-T1 and G1-T2 target sequences (FIG. 13B). Having both strands of the universal elements labelled with fluorescent dyes results in melting peaks with heights which are greater than the sum of the peaks generated with G1-JP and G1-JPrc probes used in combination with unlabelled complement oligonucleotides (FIG. 13C). DNA quenching and direct dye-dye quenching interactions within and between universal element strands causes the fluorescence reduction when the tribrid structure is formed. Dissociation of the tribrid structure results in fluorescence increases and consequently inverted melting peaks when using—dF/dT plots. Data could be presented as positive peaks using dF/dT analysis.

TABLE 16
Oligonucleotide sequences, where F is
fluorescein dT

| OLIGO | SEQUENCE | SEQ ID: |
|---|---|---|
| G1-JPrc | CCAFCCFAACAGCTTCGAAAAAGTTTTTCTTT | 107 |
| | AC | |
| G1-UCrc | CCATAACAGCAAGGTTAGGATGGTG | 108 |

### Example 15

The previous examples describe interactions between three DNA sequences to form a tribrid structure comprising a junction probe, a universal complement oligonucleotide and a target sequence. This allows the use of probes that comprise the same universal sequence element for all targets, but requires the synthesis of a new probe for each application to include a target specific element.

It is possible to create a truly universal probe if two different universal complement oligonucleotides are used to create a four-strand cruciform structure (FIG. 14). The two universal complement oligonucleotides hybridise to adjacent regions of target sequence and possess universal element sequences which are not complementary to each other. The junction probe of this embodiment has two universal elements which are complementary to the first and second UC oligonucleotides. The junction probe does not share sequence with the target and cannot hybridise in the absence of the two UC oligonucleotides.

To demonstrate the universal nature of this four-strand embodiment, the P2-JPv2 probe (Table 6) was used to detect the targets of the G1-JPv2 probe. The P2-JPv2 probe was used with the G1-UCv2 and the CRUX-UC (Table 17) complement oligonucleotides. The four strand structure formed with target sequence (FIG. 14) generates high quality melting peaks which allow detection and discrimination of the fully complementary G1-T1 and mismatched G1-T2 targets (Table 5).

Melting curve analysis was performed with 150 nM of the P2-JPv2, G1-UCv2, CRUX-UC and target oligonucleotides. The fully complementary G1-T1 and mismatched G1-T2 target sequences were clearly detected and differentiated on the basis of melting peak Tm (FIG. 15A) Melting peaks were not generated in the absence of target or when ether of the universal complement oligonucleotides was omitted, demonstrating the requirement for the formation of the four-strand cruciform structure for target sequence detection.

The requirement for hybridisation to both universal complement oligonucleotides means that multiplex analysis could be performed with the same probe sequence if oligonucleotides were labelled with different fluorophores, for example Fluorescein to detect target 1 and Texas Red to detect target 2. Alternatively, multiplex probes could comprise different universal elements or share only one of the universal element sequences.

TABLE 17
Universal complement oligonucleotide used
for four-strand sequence analysis, where 7 is
the PCR blocker 3′ amino C7

| OLIGO | SEQUENCE | SEQ ID: |
|---|---|---|
| CRUX-UC | CCATAACAGCAAGAATCGATCCCC7 | 109 |

Only one of the universal complement oligonucleotides absolutely requires a 3′ blocking modification to prevent extension during amplification, but the preference would be to have both UC oligonucleotides and junction probe capped with 3′ modification to prevent extension in the event of dimer formation.

The compatibility of the 4-strand cruciform structure with PCR-amplified sequences was demonstrated using the P2-JPv2 probe to detect the targets of the G1-JPv2 probe. Target sequences were amplified using the G1-LF and G1-LR primers (Table 7) and detection was achieved using the P2-JPv2 probe with G1-UCv2 and CRUX-UC oligonucleotides. PCR reaction volumes were 20 μl, comprising 2 μl sample, 1× Phire buffer (Fisher Scientific, UK), 1 mM dNTPs, 3 mM MgCl₂, 30 ng/μl BSA, 1 μM G1-LF primer, 222 nM G1-LR primer, 75 nM P2-JPv2 probe, 500 nM G1-UCv2 oligo, 500 nM CRUX-UC oligo and 0.8 μl Phire polymerase. PCR amplification was performed using a CFX96 thermal protocol, where following an initial denaturation step (98° C. 1 minute), targets were amplified using 50 cycles comprising denaturation (98° C. 5 seconds) and annealing/extension (65° C. 10 seconds). Melting curve analysis was performed immediately after amplification by briefly denaturing (98° C. 30 seconds) and cooling (20° C. 30 seconds) samples prior to increasing temperature from 20° C. to 70° C. in 0.5° C. steps.

High quality melting peak data was generated with the four-strand cruciform design, where the probe does not interact directly with the amplified target sequence (FIG. 15B). The sensitivity of the test was not compromised using the four-strand design achieving detection down to 1.8 target copies as demonstrated in example 7 with the standard HyBeacon® probe G1-L and the tribrid design using G1-JPv2.

### Example 16

The four strand cruciform method was tested further using the target sequences of the JP-2 probe (Table 12). The P2-JPv2 probe (Table 6) was used to detect PCR-amplified target sequences using the 1661-UC1 and 1661-UC2 oligonucleotides (Table 18) to form the four-strand structure. Target sequences were amplified using the 1661-F and 1661-R primers (Table 18) and comprised sequence of the CYP2D6 gene and the 1661 G>C (rs61736512) single nucleotide polymorphism. The P2-JPv2 probe does not bind to the CYP2D6 target sequence directly, but formation of the four-strand cruciform structure allows target detection and discrimination of 1661 G>C polymorphic sequences.

PCR reaction volumes were 20 μl, comprising 2 μl sample, 1× Phire buffer (Fisher Scientific, UK), 1 mM dNTPs, 2 mM MgCl₂, 30 ng/μl BSA, 5% DMSO, 222 nM 1661-F primer, 1 μM 1661-R primer, 150 nM P2-JPv2 probe, 500 nM 1661-UC1 oligo, 500 nM 1661-UC2 oligo and 0.8 μl Phire polymerase. PCR amplification was performed using a CFX96 thermal protocol, where following an initial denaturation step (98° C. 1 minute), targets were amplified using 50 cycles comprising denaturation (98° C. 5 seconds) and annealing/extension (65° C. 10 seconds). Melting curve analysis was performed immediately after amplification by briefly denaturing (98° C. 30 seconds) and cooling (35° C. 30 seconds) samples prior to increasing temperature from 35° C. to 60° C. in 0.5° C. steps.

The 1661-UC2 oligonucleotide hybridises to the section of the CYP2D6 gene containing the 1661 G>C polymorphism. The stability of the 1661-UC2 oligonucleotide determines the melting temperature of the four-strand cruciform structure and thereby the melting peak Tm of the P2-JPv2 probe. The 1661-UC2 oligo is fully complementary with the C allele of the rs61736512 SNP and has a position of mismatch when hybridised with the G allele. The melting peak Tms generated with C and G alleles are approximately 46° C. and 41° C., respectively (FIG. 16A). Heterozygous samples possessing both C and G alleles generate both 46° C. and 41° C. melting peaks, whereas no template controls yield neither peak.

TABLE 18
Universal complement oligonucleotides and
primers used for four-strand sequence analysis,
where p and 7 are the PCR blockers
3′ phosphate and 3′ amino C7

| OLIGO | SEQUENCE | SEQ ID: |
|---|---|---|
| 1661-UC1 | CACCATCCTAACTAACCTTGCGCAACTTGGGp | 110 |
| 1661-UC2 | CTTCTCCGTCTCCAATCGATCCC7 | 111 |
| 1661-F | GGGAGACCAGGGGGAGCATA | 112 |
| 1661-R | TGCCCATCACCCACCGGAG | 113 |

Analysis of the 1661 G>C polymorphism can also be achieved with a tribrid structure using 75 nM of the JP-2 and 500 nM of UC-2 oligonucleotides from example 11. The JP-2 probe hybridises to the CYP2D6 target at the point of the rs61736512 SNP and yields melting peak Tms of approximately 48° C. and 38° C. with C and G alleles, respectively (FIG. 16B).

### Example 17

The CXC-T3 target sequence (table 4) possesses an additional nucleotide at the tribrid junction that causes junction probe and UC oligonucleotides to be separated when hybridised to their respective target sequences. The single base separating JP and UC on the target sequence does not disrupt the formation of the tribrid structure considerably and high quality melting peaks were generated.

A further study of intervening target sequence was performed using the G1-JPv2 probe and G1-UCv2 complement oligonucleotide (Table 6). The G1-1nt, G1-2nt, G1-3nt, G1-5nt, G1-10nt and G1-20nt possess 1, 2, 3, 5, 10 and 20 additional nucleotides, respectively, at the tribrid junction to separate the target sequences of the junction probe and universal complement (Table 19).

Melting curve analysis was performed using 75 nM G1-JPv2 probe, 500 nM G1-UCv2 oligonucleotide and 1 μM of target oligonucleotide. Analysis was also performed using 150 nM of each oligonucleotide. Analysis of the target oligonucleotides detailed in table 19 was performed along with the G1-T1 target (Table 5) for comparison.

Melting peaks were generated using target sequences with 1, 2, 3 and 5 nucleotides separating JP and UC elements, but clear peaks were not observed with targets possessing 10 and 20 nucleotides of intervening sequence (FIG. 17A). Larger melting peaks were generated with 2 and 3 nucleotides of intervening sequence than when JP and UC targets were separated by 1 and 5 nucleotides.

TABLE 19
Oligonucleotide targets with additional sequence
separating junction probe and universal
complement elements

| | | SEQ |
|---|---|---|
| OLIGO | SEQUENCE | ID: |
| G1-1nt | GTAAAGAAAACTTTTTCGAAGCTACTTGCTGTTATGG | 114 |
| G1-2nt | GTAAAGAAAACTTTTTCGAAGCTAACTTGCTGTTAT | 115 |
| | GG | |
| G1-3nt | GTAAAGAAAACTTTTTCGAAGCTAAACTTGCTGTTA | 116 |
| | TGG | |
| G1-5nt | GTAAAGAAAACTTTTTCGAAGCTAAAAACTTGCTGT | 117 |
| | TATGG | |
| G1-10nt | GTAAAGAAAACTTTTTCGAAGCTAAAAAAAAAACTT | 118 |
| | GCTGTTATGG | |
| G1-20nt | GTAAAGAAAACTTTTTCGAAGCTAAAAAAAAAAAA | 119 |
| | AAAAAAAACTTGCTGTTATGG | |

The target oligonucleotides detailed in table 19 were also used to evaluate the four-strand cruciform structure described in example 15. The P2-JPv2 probe was able to detect the targets with 1, 2, 3, 5 and 10 nucleotides of intervening sequence (FIG. 17B).

The melting peaks generated with the 3-strand tribrid structure were all of similar Tm to the standard G1-T1 target sequence, whereas the peaks generated with the four-strand structure exhibited decreasing Tms as the length of intervening sequence increased. The tribrid structure is likely to be more flexible with the target sequence able to form “loops” or “bubbles” to bring JP and UC oligonucleotides into closer proximity, The four-strand cruciform structure is likely to be less flexible, where intervening sequence at the junction will cause destabilisation of UC oligonucleotide interactions with the target sequence. This Tm reduction may be a useful feature for the detection of sequence insertions and deletions.

### Example 18

The method described in WO 2009/053679 is applied here to differentiate target sequences on the basis of length using a four-strand universal structure.

The P2-JPv2 probe (Table 6) was used to indirectly detect the Short Tandem Repeat (STR) targets of the D16S539 locus, using the D16-BL-UC and D16-P-UC oligonucleotides to hybridise with probe and target sequences. The D16-BL-UC oligonucleotide acts as a blocker when used at a molar excess to limit the length of D16-P-UC hybridisation. The blocking D16-BL-UC and probing D16-P-UC oligonucleotides both comprise six [TATC] repeats. All of the D16-P-UC repeats are hybridised with target sequences possessing twelve repeats of [GATA], but the number of D16-P-UC repeats hybridised is reduced with shorter STR targets. The stability of the D16-P-UC oligonucleotide determines the melting temperature of the four-strand cruciform structure and thereby the melting peak Tm of the P2-JPv2 probe. The ability to differentiate targets on the basis of length was evaluated by melting curve analysis using oligonucleotide target sequences possessing 10, 11, 12 and 13 repeats of the [GATA] sequence (Table 20). Formation of the cruciform structure with 10, 11 and 12 repeat targets generated melting peaks with Tms of 36.5° C., 39.5° C. and 42.5° C., respectively (FIG. 18). Formation of the cruciform structure with a 13 repeat target sequence yields a melting peak with a Tm of 36.0° C., where the additional repeat in the target sequence forms a loop and causes destabilisation of the junction.

This example demonstrates the proof-of-principle for the analysis of length polymorphisms using the universal method and highlights the design considerations required for oligonucleotide synthesis.

TABLE 20
Universal complement and STR target
oligonucleotides

| OLIGO | SEQUENCE | SEQ ID: |
|---|---|---|
| D16-BL-UC | CATCCTAACTA(TATC)₆ACCTGTCTG | 120 |
| D16-P-UC | CAATGA(TATC)₆AATCGATCCCC | 121 |
| D16-10rpt | CAGACAGGTG(GATA)₁₀TCATTG | 122 |
| D16-11rpt | CAGACAGGTG(GATA)₁₁TCATTG | 123 |
| D16-12rpt | CAGACAGGTG(GATA)₁₂TCATTG | 124 |
| D16-13rpt | CAGACAGGTG(GATA)₁₃TCATTG | 125 |

The method detailed in WO 2009/053679 describes an oligonucleotide that combines one of the primers with the non-fluorescent blocking oligonucleotide. This method could also be applied here to combine one of the primers with one of the universal complement oligonucleotides. The universal complement oligonucleotide would hybridise to amplified target sequence as part of a unimolecular structure (i.e. part of the same DNA strand). The concept of the unimolecular UC oligonucleotide is compatible with three-strand tribrid and four-strand cruciform structures used for universal analysis (FIG. 19).

### Example 19

The compatibility of the universal methods of sequence analysis was evaluated with non-HyBeacon® oligonucleotide probes. Simple probes (U.S. Pat. No. 6,635,427) labelled on 5′ or 3′ ends were tested with the three-strand (tribrid) and four-strand (cruciform) universal structures (Table 21). Melting curve analysis was performed with the oligonucleotide target sequences G1-T1, G1-T2, P2-T1 and P2-T2 (Table 5). Probes, UC oligonucleotides and target oligonucleotides were all used at 150 nM with melting curve analysis performed between 20° C. and 70° C.

The probe labelled on the 5′ end was tested with the four-strand universal method. Positive melting peaks were generated when the P2-5JP probe was used with:
G1-UCv2 (Table 6) and CRUX-UC (Table 17) in a cruciform format;
G1-UCv2 and CRUX-UCv2 (Table 21) in a cruciform format (FIG. 20A).

The probe labelled on the 3′ end was tested with both the three-strand and four-strand universal methods. The P2-3JP labelled probe generated positive and inverted melting peaks depending on the length of the UC oligonucleotide used:
Small positive melting peaks were generated with G1-UCv2 and CRUX-UC in the cruciform format, where the length of the universal element sequence in the probe exceeds the UC.
Inverted peaks were generated with G1-UCv3 and CRUX-UC in the cruciform format, where the length of the universal elements in probe and UC were equal.
Positive melting peaks were generated with G1-UCv4 and CRUX-UC in the cruciform format, where the length of the universal element sequence in the UC oligonucleotide exceeds the length of the probe.
Inverted peaks were again generated with the P2-3JP probe when used with P2-UCv3 (Table 21), where the length of the universal elements in probe and UC were equal (FIG. 20B).
Positive melting peaks were generated with the P2-3JP when used with P2-UCv4 (Table 21), when the length of the universal element sequence in the UC oligonucleotide exceeds the length of the probe (FIG. 20C).

The fluorescence emission of dyes attached to terminal nucleotides is influenced more by neighbouring bases and target sequence than the internal dye-labels used in HyBeacon® probes. In an extreme example, the direction of fluorescence change in terminally labelled probes can switch from positive to negative peaks when detecting the sequence variants of a biallelic SNP.

This example demonstrates compatibility of 5′ and 3′ labelled probes with the three-strand and four-strand universal methods. Any oligonucleotide probe technology that uses melting curve analysis for sequence detection and discrimination should be suitable for universal analysis.

TABLE 21
Oligonucleotides to test probes labelled on
5′ or 3′ termini, where 5′F and 3′F are
fluorescein labels attached to 5′ and 3′ ends.

| | | SEQ |
|---|---|---|
| OLIGO | SEQUENCE | ID: |
| P2-5JP | [5′F]GGGGATCGATTTAGTTAGGATGGTG | 126 |
| P2-3JP | GGGGATCGATTTAGTTAGGATGGTG[3′Flc] | 127 |
| G1-UCv3 | CACCATCCTAACTAAGCTTCGAAAAAGTTTTCTTT | 128 |
| | AC | |
| G1-UCv4 | AAACACCATCCTAACTAAGCTTCGAAAAAGTTTTC | 129 |
| | TTTAC | |
| CRUX-UCv2 | CCATAACAGCAAGAATCGATCCCCAAA | 130 |
| P2-UCv3 | CACCATCCTAACTAGAAACTCTTTTTGTAGAGTTTG | 131 |
| P2-UCv4 | AAACACCATCCTAACTAGAAACTCTTTTTGTAGAG | 132 |
| | TTTG | |

### Example 20

When designing junction probes and universal complements, oligonucleotide melting temperatures were determined using nearest neighbour thermodynamic calculations (Breslauer et al. 1986; SantaLucia, 1998).

The melting temperature (Tₘ) of hybridised universal element sequence (oligonucleotide sections 1 & 3 in FIG. 1) is preferably between 5° C. and 30° C. to prevent interaction of junction probe and universal complement oligonucleotides in the absence of target sequence. Either the probe or universal complement oligonucleotide may form an overhang when universal element sequences are hybridised. Alternatively, the universal element sequences of junction probe and universal complement oligonucleotides may be of identical length.

The Tₘ of the probe target specific element (oligonucleotide section 2 in FIG. 1) is preferably between 25° C. and 50° C. More preferably, the Tₘ of the target specific element is between 30° C. and 40° C.

The Tₘ of the universal complement target specific element (oligonucleotide section 4 in FIG. 1) is preferably between 45° C. and 70° C. More preferably, the Tₘ of the target specific element is between 50° C. and 60° C. The Tₘ of the UC target specific sequence is typically designed to be higher than the Tₘ of the probe target specific sequence to account for unknown polymorphisms in the UC target sequence and to ensure that melting peaks are determined by probe dissociation rather than UC dissociation.

These design guidelines also apply to application of the four-strand structure, where the junction probes of this embodiment have two universal elements but no target specific sequence. The two universal complement oligonucleotides each comprise a target specific element and a universal sequence element complementary to a portion of the junction probe.

Oligonucleotide ΔG (Gibbs free energy) is defined as the net exchange of energy between the system and environment. The following calculation can be used to determine oligonucleotide ΔG:

ΔG=ΔH−T·ΔS

Where ΔH is the enthalpy (total energy exchange between the system and environment), ΔS=entropy (the energy spent by the system to organise itself) and T=temperature (degrees Kelvin). For oligonucleotides, positive AG values indicate that the system will move in the direction of the single strand, whereas negative AG values indicate that the system will move in the direction of the product (i.e. probe/target duplex or oligonucleotide secondary structure). The system is in equilibrium when ΔG=0 kcal/mole and this is generally the Tₘ of the system where 50% of the oligonucleotide is single-stranded and 50% is hybridised.

Negative ΔG values provide an indication of oligonucleotide secondary structure and the magnitude can determine the performance of primers and probes. For example, primers which have 3′ ends with ΔG values that are more negative than −3 kcal/mol should be avoided to allow efficient extension, whereas an overall ΔG of up to −9 kcal/mol can be tolerated (Rychlik, 1995).

Probes with intramolecular secondary structures can generate their own melting peaks in the absence of target sequence and UC oligonucleotide. Universal complement oligonucleotides with strong secondary structures may be more likely to form the unimolecular species rather than hybridise with the universal element of the probe. To avoid problematic secondary structures, the ΔG values of probe and UC oligonucleotides are preferably greater (more positive) than −5.0 kcal/mole. More preferably the ΔG values are greater (more positive) than −3.0 kcal/mole. ΔG values are calculated with the mfold program (Zuker, 2003) at 37° C.

### REFERENCES

1. Sinha, N. D., Biernat, J., McManus, J. and Koster, H. (1984) Polymer support oligonucleotide synthesis. 18. Use of beta-cyanoethyl-N,N-dialkylamino-/N-morpholino phosphoramidite of deoxynucleosides for the synthesis of DNA fragments simplifying deprotection and isolation of the final product. Nucleic Acids Res., 12, 4539-4557.
2. Sinha, N. D., Biernat, J. and Koster, H. (1983) Beta-cyanoethyl-N,N-dialkylamino-/N-morpholino phosphoramidites, new phosphitylating agents facilitating ease of deprotection and work-up of synthesized oligonucleotides. Tetrahedron Lett., 24, 5843-5846.
3. Beaucage, S. L. and Iyer, R. P. (1993) The synthesis of oligonucleotides by the phosphoramidite approach and their applications. Tetrahedron, 49, 6123-6194.
4. Beaucage, S. L. and Iyer, R. P. (1992) Advances in the synthesis of oligonucleotides by the phosphoramidite approach. Tetrahedron, 48, 2223-2311.
5. Caruthers, M. H., Barone, A. D., Beaucage, S. L., Dodds, D. R., Fisher, E. F., McBride, L. J., Matteucci, M., Stabinsky, Z. and Tang, J. Y. (1987) Chemical Synthesis of deoxyoligonucleotides by the phosphoramidite method. Method Enzymol., 154, 287-313.
6. Gait, M. J., Singh, M., Sheppard, R. C., Edge, M. D., Greene, A. R., Heathcliffe, G. R., Atkinson, T. C., Newton, C. R. and Markham, A. F. (1980) Rapid synthesis of oligodeoxyribonucleotides 4. Improved solid-phase synthesis of oligodeoxyribonucleotides through phosphotriester intermediates. Nucleic Acids Res., 8, 1081-1096.
7. Jankowska, J., Sobkowski, M., Stawinski, J. and Kraszewski, A. (1994) Studies on aryl-H-phosphonates 1. An efficient method for the preparation of deoxyribonucleosides and ribonucleoside 3′-H-phosphonate monoesters by transesterification of deiphenyl-H-phosphonate. Tetrahedron Lett., 35, 3355-3358.
8. Matteucci, M. D. and Caruthers, M. H. (1981) Nucleotide chemistry 4. Synthesis of deoxyoligonucleotides on a polymer support. J. Am. Chem. Soc., 103, 3185-3191.
9. Reverse-synthesis: 5′- to 3′-oligonucleotide synthesis: http://www.glenresearch.com/GlenReports/GR6-14.html.
10. G.E.-Healthcare. AKTA OligoPilot: http://www.qelifesciences.com/webapp/wcs/stores/servlet/productByld/en/GELifeSciences-UK/18114042.
11. Englisch, U. and Gauss, D. H. (1991) Chemically modified oligonucleotides as probes and inhibitors. Angew. Chem. Int. Edit., 30, 613-629.
12. Shelbourne, M., Brown, T. and El-Sagheer, A. H. (2012) Fast and efficient DNA crosslinking and multiple orthogonal labelling by copper-free click chemistry. Chem. Commun., 48, 11184-11186.
13. Fruk, L., Grondin, A., Smith, W. E. and Graham, D. (2002) A new approach to oligonucleotide labelling using Diels-Alder cycloadditions and detection by SERRS. Chem. Commun., 2100-2101.
14. El-Sagheer, A. H. and Brown, T. (2010) Click chemistry with DNA. Chem. Soc. Rev., 39, 1388-1405.
15. Brown, T. and Brown, D. J. S. (1992) Purification of Synthetic DNA. Method Enzymol., 211, 20-35.
16. Ranasinghe, R. T. and Brown, T. (2011) Ultrasensitive fluorescence-based methods for nucleic acid detection: towards amplification-free genetic analysis. Chem. Commun., 47, 3717-3735.
17. Narayanan, S., Gall, J. and Richert, C. (2004) Clamping down on weak terminal base pairs: oligonucleotides with molecular caps as fidelity-enhancing elements at the 5′- and 3′-terminal residues. Nucleic Acids Res., 32, 2901-2911.
18. Dogan, Z., Paulini, R., Stutz, J. A. R., Narayanan, S. and Richert, C. (2004) 5′-Tethered stilbene derivatives as fidelity- and affinity-enhancing modulators of DNA duplex stability. J. Am. Chem. Soc., 126, 4762-4763.
19. Patra, A. and Richert, C. (2009) High Fidelity Base Pairing at the 3′-Terminus. J. Am. Chem. Soc., 131, 12671-12681.
20. Breslauer et al. (1986) Predicting DNA duplex stability from the base sequence. Proc. Nat. Acad. Sci. 83:3746-50.
21. Gerrard, S. R., Hardiman, C., Shelbourne, M., Nandhakumar, I., Norden, B. and Brown, T. (2012) A New Modular Approach to Nanoassembly: Stable and Addressable DNA Nanoconstructs via Orthogonal Click Chemistries. Acs Nano, 6, 9221-9228.
22. Shelbourne, M., Chen, X., Brown, T. and El-Sagheer, A. H. (2011) Fast copper-free click DNA ligation by the ring-strain promoted alkyne-azide cycloaddition reaction. Chem. Commun., 47, 6257-6259.
23. El-Sagheer, A. H. and Brown, T. (2012) Click Nucleic Acid Ligation: Applications in Biology and Nanotechnology. Accounts Chem. Res., 45, 1258-1267.
24. El-Sagheer, A. H., Sanzone, A. P., Gao, R., Tavassoli, A. and Brown, T. (2011) Biocompatible artificial DNA linker that is read through by DNA polymerases and is functional in Escherichia coli. Proc. Natl. Acad. Sci. U.S.A., 108, 11338-11343.
25. El-Sagheer, A. H. and Brown, T. (2009) Synthesis and Polymerase Chain Reaction Amplification of DNA Strands Containing an Unnatural Triazole Linkage. J. Am. Chem. Soc., 131, 3958-3964.
26. Kumar, R., El-Sagheer, A. H., Tumpane, J., Lincoln, P., Wilhelmsson, L. M. and Brown, T. (2007) Template-directed oligonucleotide strand ligation, covalent intramolecular DNA circularization and catenation using click chemistry. J. Am. Chem. Soc., 129, 6859-6864.
27. El-Sagheer, A. H., Cheong, V. V. and Brown, T. (2011) Rapid chemical ligation of oligonucleotides by the Diels-Alder reaction. Org. Biomol. Chem., 9, 232-235.
28. Didenko (2001) DNA probes using Fluorescence Resonance Energy Transfer (FRET): Designs and applications Biotechniques, 31, 1106-1121.
29. French et al. (2001) HyBeacon™ probes: a new tool for DNA sequence detection and allele discrimination. Molecular and Cellular Probes, 15, 363-374.
30. French et al. (2002) Ultra-rapid DNA analysis using HyBeacon®™ probes and direct PCR amplification from saliva. Molecular and Cellular probes 16, 319-326.
31. French et al. (2007) Analysis of multiple single nucleotide polymorphisms closely positioned in the ovine PRNP gene using linear fluorescent probes and melting curve analysis. BMC Infectious Diseases 7:90.
32. French et al. (2008) Interrogation of short tandem repeats using fluorescent probes and melting curve analysis: a step towards rapid DNA identity screening. Forensic Sci Int Genet. 4, 333-339.
33. Ben Gaied et al. (2010) End-capped HyBeacon@ probes for the analysis of human genetic polymorphisms related to warfarin metabolism. Org Biomol Chem 12, 2728-34.
34. Notomi et al. (2000) Loop-mediated isothermal amplification of DNA. Nucleic Acids Res. June 15; 28(12):E63.
35. Rychlik W. (1995) Selection of primers for polymerase chain reaction. Mol Biotechnol. April; 3(2):129-34.
36. SantaLucia, Jr (1998) A unified view of polymer, dumbbell, and oligonucleotide DNA nearest-neighbor thermodynamics. Proc. Natl. Acad. Sci. USA 95, 1460-1465).
37. Zuker M. (2003) Mfold web server for nucleic acid folding and hybridization prediction. Nucleic Acids Res. July 1; 31(13):3406-15.

| | | |
|---|---|---|
| WO 01/73118 | October 2001 | French et al. |
| WO 07/010268 | January 2007 | French et al. |
| WO 09/053679 | April 2009 | Gale et al. |
| US 2011/0212540 | September 2011 | Hsin-Chih Yeh et al. |
| US 2009/0176318 | July 2009 | Kolpashchikov |
| U.S. Pat. No. 6,635,427 | October 2003 | Wittwer et al. |

1. A method of detecting the presence of a target polynucleotide and/or sequence variations within the target polynucleotide in a sample of interest, comprising the steps of:
(i) providing a probe comprising two independent partner oligonucleotide components comprising:
a) a first oligonucleotide having a first and second section, wherein the first section comprises a nucleotide sequence that is labelled with at least one visually detectable label and is not capable of hybridising to the nucleotide sequence of the target polynucleotide; and wherein the second section comprises a nucleotide sequence that is capable of hybridising to a portion of the sequence of the target polynucleotide said second section of the first oligonucleotide having a melting temperature (Tm) of between 25 and 50° C.; and
b) a second oligonucleotide having a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to said second section of the second oligonucleotide having a melting temperature (Tm) of between 40 and 70° C.;
(ii) exposing the sample of interest to the probe of (i), such that in the presence of the target polynucleotide, a triple hybridisation event can occur which involves (a) the hybridisation of the first section of the first oligonucleotide to the first section of the second oligonucleotide; and (b) the hybridisation of both the second section of the first oligonucleotide and the second section of the second oligonucleotide to the target polynucleotide;
(iii) detecting a change in the visually detectable label, wherein a change in the visually detectable label indicates the presence of the target polynucleotide and/or the sequence variations within the target polynucleotide; and
(iv) analysing the change in the visually detectable label using either melting curve analysis or annealing curve analysis.

2. The method of claim 1 wherein the first oligonucleotide is labelled internally with the at least one visually detectable label.

3. The method of either claim 1 or 2 wherein the the first oligonucleotide is between 15 and 40 or between 20 and 30 nucleotide residues in length; and/or the second oligonucleotide is between 20 and 50 or 25 and 40 nucleotide residues in length.

4. The method of any previous claim wherein the nucleotide sequence of the target polynucleotide to which the second sections of the first and second oligonucleotides hybridise are between 15 and 50 or 25 and 40 nucleotides in length.

5. The method of any previous claim wherein the target polynucleotide and/or the sequence variations within the target polynucleotide are only detectable after the triple hybridisation event has occurred.

6. The method of any previous claim wherein the second section of the first oligonucleotide and the second section of the second oligonucleotide hybridise to contiguous sequences of the target polynucleotide.

7. The method of any of claims 1 to 5 wherein the second section of the first oligonucleotide and the second section of the second oligonucleotide hybridise to non-contiguous sequences of the target polynucleotide.

8. The method of any previous claim wherein the melting temperature (Tₘ) of the second section of the first oligonucleotide is between 30° C. and 40° C.; and the Tₘ of the second section of the second oligonucleotide is between 50° C. and 60° C.

9. The method of any previous claim wherein the melting temperature (Tₘ) of the target polynucleotide of the second section of the second oligonucleotide is greater than the Tₘ of the target polynucleotide of the second section of the first oligonucleotide to ensure identification of mismatched sequences.

10. The method of claim 9 wherein the Tₘ of the second section of the second oligonucleotide is 10° C. higher than the second section of the first oligonucleotide.

11. The method of any of claims 1 to 8 wherein the Tₘ of the target polynucleotide of the second section of the second oligonucleotide is the same as the Tₘ of the target polynucleotide of the second section of the first oligonucleotide.

12. A method of detecting the presence of a target polynucleotide and/or sequence variations within the target polynucleotide in a sample of interest, comprising the steps of:
(i) providing a probe system comprising three independent partner oligonucleotide components comprising:
a) a first oligonucleotide having a first and second section, wherein the first oligonucleotide is not capable of hybridising to the sequence of the target polynucleotide and wherein the first section comprises a nucleotide sequence that is labelled with at least one visually detectable label that is capable of hybridising to a portion of the sequence of a second oligonucleotide; and wherein the second section comprises a nucleotide sequence that is capable of hybridising to a portion of the sequence of a third oligonucleotide, said second section of the first oligonucleotide having a melting temperature (Tm) of between 40 and 70° C.;
b) a second oligonucleotide having a first and second section, wherein the first section comprises a nucleotide sequence which is capable of hybridising to a nucleotide sequence of the first section of the first oligonucleotide; and the second section comprises a nucleotide sequence which is capable of hybridising to a nucleotide sequence of the target polynucleotide, said second section of the second oligonucleotide having a melting temperature (Tm) of between 25 and 50° C.; and
c) a third oligonucleotide having a first and second section, wherein the first section comprises a nucleotide sequence which is capable of hybridising to a nucleotide sequence of the second section of the first oligonucleotide; and the second section comprises a nucleotide sequence which is capable of hybridising to a nucleotide sequence of the target polynucleotide that is adjacent to the nucleotide sequence that the second section of the second oligonucleotide is complementary to, said second section of the third oligonucleotide having a melting temperature (Tm) of between 40 and 70° C.;
(ii) exposing the sample of interest to the probe system of (i), such that in the presence of the target polynucleotide, a quadruple hybridisation event can occur which involves (a) the hybridisation of the first section of the first oligonucleotide to the first section of the second oligonucleotide; and (b) the hybridisation of the second section of the first oligonucleotide to the first section of the third oligonucleotide; and (c) the hybridisation of both the second section of the second oligonucleotide and the second section of the third oligonucleotide to the target polynucleotide;
(iii) detecting a change in the visually detectable label, wherein a change in the visually detectable label indicates the presence of the target polynucleotide and/or the sequence variations within the target polynucleotide; and
(iv) analysing the change in the visually detectable label using either melting curve analysis or annealing curve analysis.

13. The method of claim 12 wherein the first oligonucleotide is labelled internally with the at least one visually detectable label.

14. The method of either claim 12 or 13 wherein the first oligonucleotide is between 18 and 35 or 20 and 30 nucleotide residues in length; and/or the second oligonucleotide is between 15 and 40 or between 20 and 30 nucleotide residues in length; and/or the third oligonucleotide is between 20 and 50 or 25 and 40 nucleotide residues in length.

15. The method of any of claims 12 to 14 wherein the nucleotide sequence of the target polynucleotide to which the second sections of the second and third oligonucleotides hybridise are between 15 and 50 or 25 and 40 nucleotides in length.

16. The method of any of claims 12 to 15, wherein the target polynucleotide and/or the sequence variations within the target polynucleotide are only detectable after the quadruple hybridisation event has occurred.

17. The method of any of claims 12 to 16 wherein the second section of the first oligonucleotide and the second section of the third oligonucleotide hybridise to contiguous sequences of the target polynucleotide.

18. The method of any of claims 12 to 16 wherein the second section of the first oligonucleotide and the second section of the third oligonucleotide hybridise to non-contiguous sequences of the target polynucleotide.

19. The method of any of claims 12 to 18 wherein the melting temperature (Tₘ) of the second section of the second oligonucleotide is between 30° C. and 40° C.; and/or the Tₘ of the second section of the third oligonucleotide is between 50° C. and 60° C. and/or the Tₘ of the first oligonucleotide is between 50° C. and 60° C.

20. The method of any of claims 12 to 10 wherein the Tₘ of the target polynucleotide of the second section of the third oligonucleotide is greater than the Tₘ of the target polynucleotide of the second section of the first oligonucleotide to ensure identification of mismatched sequences.

21. The method of claim 20 wherein the Tₘ of the second section of the third oligonucleotide is 10° C. higher than the second section of the first oligonucleotide.

22. The method of any of claims of claims 12 to 19 wherein the Tₘ of the target polynucleotide of the second section of the third oligonucleotide is the same as the Tₘ of the target polynucleotide of the second section of the first oligonucleotide.

23. The method of any previous claim wherein the first and second sections of the first oligonucleotide are joined at a junction, said junction being positioned at least two nucleotides from the visually detectable label positioned 5′-most when the first oligonucleotide has been synthesised in the 3′ to 5′ direction; or said junction being positioned at least two nucleotides from the visually detectable label positioned 3′-most when the first oligonucleotide has been synthesised in the 5′ to 3′ direction.

24. The method of claim 23 wherein the junction is positioned at least three nucleotides from the visually detectable label positioned 5′-most when the first oligonucleotide has been synthesised in the 3′ to 5′ direction; or said junction being positioned at least three nucleotides from the visually detectable label positioned 3′-most when the first oligonucleotide has been synthesised in the 5′ to 3′ direction.

25. The method of claim 23 or 24 wherein the junction is positioned at least four nucleotides from the visually detectable label positioned 5′-most, wherein the first oligonucleotide has been synthesised in the 3′ to 5′ direction; or said junction being positioned at least four nucleotides from the visually detectable label positioned 3′-most, wherein the first oligonucleotide has been synthesised in the 5′ to 3′ direction.

26. The method of any previous claim wherein the junction is positioned two, three or four nucleotides from the visually detectable label positioned 5′-most when the first oligonucleotide has been synthesised in the 3′ to 5′ direction; or said junction being positioned at least two or three or four nucleotides from the visually detectable label positioned 3′-most when the first oligonucleotide has been synthesised in the 5′ to 3′ direction.

27. The method of any of claims 1 to 22 wherein the first and second sections of the first oligonucleotide are joined at a junction, said junction being positioned one nucleotide from the visually detectable label positioned 5′-most when the first oligonucleotide has been synthesised in the 3′ to 5′ direction; or said junction being positioned at one nucleotide from the visually detectable label positioned 3′-most when the first oligonucleotide has been synthesised in the 5′ to 3′ direction

28. The method of any previous claim wherein the independent partner oligonucleotides are not labelled with a quencher.

29. The method of any previous claim wherein the hybridisation of the first section of the first oligonucleotide to the first section of the second oligonucleotide improves the detection of the visually detectable label.

30. The method of any previous claim wherein the first section of the second oligonucleotide is also labelled with a visually detectable label.

31. The method of any of claims 12 to 30 wherein the first section of the third oligonucleotide is also labelled with a visually detectable label.

32. The method of any previous claim wherein the visually detectable label is a fluorophore or a dye.

33. The method of any previous claim wherein the visually detectable label is fluorescein dT.

34. The method of claim 32 wherein the first section of the first oligonucleotide is labelled internally with two or more; three or more; or four or more visually detectable labels.

35. The method of claim 29 wherein the improvement in detection is by an enhancement of fluorescent emissions.

36. The method of any of claims 1 to 28 wherein the hybridisation of the first section of the first oligonucleotide to the first section of the second oligonucleotide alters the emission of the visually detectable label by quenching fluorescence.

37. The method of any previous claim wherein the first section of the first oligonucleotide is longer than the first section of the second oligonucleotide resulting in an overhanging nucleotide sequence.

38. The method of any of claims 1 to 36 wherein the first section of the first oligonucleotide has the same nucleotide sequence length as the first section of the second oligonucleotide, preferably wherein the Tₘ is optimised to prevent the first section of the first oligonucleotide hybridising to the first section of the second oligonucleotide in the absence of target polynucleotide.

39. The method of any previous claim wherein the sequence variation within the target polynucleotide is a known polymorphism.

40. The method of claim 39, wherein the known polymorphism is detected by the generation of a defined melting peak Tₘ or a defined annealing peak Tₐ.

41. The method of any previous claim wherein the sequence variation within the target polynucleotide is an unknown polymorphism.

42. The method of claim 39, wherein the unknown polymorphism is detected by the generation of previously unknown melting peak Tₘ or annealing peak Tₐ.

43. The method of any of claims 36 to 42, wherein the melting or annealing curve analysis indicates the absence of the target polynucleotide due to the presence of only a melting or annealing peak specific to the hybridisation of the first and second oligonucleotides and/or the first and third oligonucleotides.

44. The method of any previous claim wherein the detection step is used in target detection, SNP genotyping, or detection of length polymorphisms and repetitive sequences.

45. The method of any previous claim wherein the target polynucleotide is a DNA or an RNA.

46. The method of any previous claim for use in conjunction with a Polymerase Chain Reaction (PCR) wherein a section of the probe constitutes a PCR primer.

47. The method of claim 46 further comprising the use of a 3′ blocking modification to prevent unwanted amplification and dimer formation during PCR.

48. The method of claim 46 wherein no 3′ blocking is required because the 3′ end of the section of the probe constituting the PCR primer is located within the first section of the first oligonucleotide.

49. The method of any previous claim for use in conjunction with an isothermal nucleic acid amplification methodology such as Loop-mediated isothermal amplification (LAMP) method, wherein a section of the probe constitutes a LAMP primer.

50. A probe system as defined in any of claims 1 to 49.

51. An oligonucleotide hybridisation structure comprising three oligonucleotides;
(a) a target polynucleotide;
(b) a first oligonucleotide having a first and second section, wherein the first section comprises a nucleotide sequence that is labelled with at least one visually detectable label and is not capable of hybridising to the nucleotide sequence of the target polynucleotide; and wherein the second section comprises a nucleotide sequence that is capable of hybridising to a portion of the sequence of the target polynucleotide, said second section of the first oligonucleotide having a melting temperature (Tm) of between 25 and 50° C.; and
(c) a second oligonucleotide having a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to, said second section of the second oligonucleotide having a melting temperature (Tm) of between 40 and 70° C.;
wherein the first section of the first oligonucleotide is hybridised to the first section of the second oligonucleotide; and both the second section of the first oligonucleotide and the second section of the second oligonucleotide are hybridised to the target polynucleotide

52. An oligonucleotide hybridisation structure comprising four oligonucleotides;
(a) a target polynucleotide;
(b) a first oligonucleotide having a first and second section, wherein the first oligonucleotide is not capable of hybridising to the sequence of the target polynucleotide and wherein the first section comprises a nucleotide sequence that is labelled with at least one visually detectable label that is capable of hybridising to a portion of the sequence of a second oligonucleotide; and wherein the second section comprises a nucleotide sequence that is capable of hybridising to a portion of the sequence of a third oligonucleotide, said second section of the first oligonucleotide having a melting temperature (Tm) of between 40 and 70° C.;
(c) a second oligonucleotide having a first and second section, wherein the first section comprises a nucleotide sequence which is capable of hybridising to a nucleotide sequence of the first section of the first oligonucleotide; and the second section comprises a nucleotide sequence which is capable of hybridising to a nucleotide sequence of the target polynucleotide, said second section of the second oligonucleotide having a melting temperature (Tm) of between 25 and 50° C.; and
(d) a third oligonucleotide having a first and second section, wherein the first section comprises a nucleotide sequence which is capable of hybridising to a nucleotide sequence of the second section of the first oligonucleotide; and the second section comprises a nucleotide sequence which is capable of hybridising to a nucleotide sequence of the target polynucleotide that is adjacent to the nucleotide sequence that the second section of the second oligonucleotide is complementary to, said second section of the third oligonucleotide having a melting temperature (Tm) of between 40 and 70° C.;
wherein the first section of the first oligonucleotide is hybridised to the first section of the second oligonucleotide; the second section of the first oligonucleotide is hybridised to the first section of the third oligonucleotide;
and both the second section of the second oligonucleotide and the second section of the third oligonucleotide are hybridised to the target polynucleotide.

53. An oligonucleotide hybridisation structure as claimed in claims 51 and 52 wherein the first oligonucleotide is labelled internally with the at least one visually detectable label.

54. An oligonucleotide hybridisation structure as claimed in claims 51 and 53 wherein the first section of the first oligonucleotide is longer than the first section of the second oligonucleotide resulting in an overhanging nucleotide sequence.

55. An oligonucleotide hybridisation structure as claimed in claims 51 to 54 wherein the second section of the first oligonucleotide and the second section of the third oligonucleotide hybridise to contiguous sequences or non-contiguous sequences of the target polynucleotide.

56. An oligonucleotide hybridisation structure as claimed in claims 51 to 55 wherein the first section of the second oligonucleotide is also labelled with a visually detectable label.

57. An oligonucleotide hybridisation structure as claimed in claims 52 to 56 wherein the first section of the third oligonucleotide is also labelled with a visually detectable label.

58. An oligonucleotide hybridisation structure as claimed in claims 51 to 57 wherein the visually detectable label is a fluorophore or a dye.

59. An oligonucleotide hybridisation structure as claimed in claims 51 to 58 wherein the visually detectable label is fluorescein dT.

60. An oligonucleotide hybridisation structure as claimed in claims 51 to 59 wherein the first section of the first oligonucleotide is labelled with two or more; three or more; or four or more visually detectable labels.

61. Use of the probe system of claim 50 in a method of detecting a target, SNP genotyping, or detecting length polymorphisms and repetitive sequences.

62. A kit of parts comprising:
(a) a first oligonucleotide having a first and second section, wherein the first section comprises a nucleotide sequence that is labelled with at least one visually detectable label and is not capable of hybridising to the nucleotide sequence of the target polynucleotide; and wherein the second section comprises a nucleotide sequence that is capable of hybridising to a portion of the sequence of the target polynucleotide, said second section of the first oligonucleotide having a melting temperature (Tm) of between 25 and 50° C.; and
(b) a second oligonucleotide having a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to, said second section of the second oligonucleotide having a melting temperature (Tm) of between 40 and 70° C.; and
(c) instructions for use.

63. A kit of parts comprising:
(a) a first oligonucleotide having a first and second section, wherein the first oligonucleotide is not capable of hybridising to the sequence of the target polynucleotide and wherein the first section comprises a nucleotide sequence that is labelled with at least one visually detectable label that is capable of hybridising to a portion of the sequence of a second oligonucleotide; and wherein the second section comprises a nucleotide sequence that is capable of hybridising to a portion of the sequence of a third oligonucleotide, said second section of the first oligonucleotide having a melting temperature (Tm) of between 40 and 70° C.;
(b) a second oligonucleotide having a first and second section, wherein the first section comprises a nucleotide sequence which is capable of hybridising to a nucleotide sequence of the first section of the first oligonucleotide; and the second section comprises a nucleotide sequence which is capable of hybridising to a nucleotide sequence of the target polynucleotide, said second section of the second oligonucleotide having a melting temperature (Tm) of between 25 and 50° C.;
(c) a third oligonucleotide having a first and second section, wherein the first section comprises a nucleotide sequence which is capable of hybridising to a nucleotide sequence of the second section of the first oligonucleotide; and the second section comprises a nucleotide sequence which is capable of hybridising to a nucleotide sequence of the target polynucleotide that is adjacent to the nucleotide sequence that the second section of the second oligonucleotide is capable of hybridising to, said second section of the third oligonucleotide having a melting temperature (Tm) of between 40 and 70° C.; and
d) instructions for use.

64. The kit of either of claim 62 or 63 wherein the kit also comprises one or more selected from reaction buffer (for PCR or isothermal amplification), dNTPs, oligonucleotide primers, enzyme and further additives including but not limited to MgCl₂, Bovine Serum Albumin (BSA), Dimethyl Sulfoxide (DMSO), Betaine, Tween-20 and carrier RNA.

65. An oligonucleotide library comprising a plurality of first oligonucleotides attached to a solid support, each first oligonucleotide comprising a first section, said first section comprising a nucleotide sequence that is labelled with at least one visually detectable label, and is capable of the nucleotide sequence being extended to include a second section, the second section of the first oligonucleotide being capable of hybridising to a target polynucleotide and the first section of the first oligonucleotide not being capable of hybridising to the nucleotide sequence of a target polynucleotide; and wherein the at least one visually detectable label undergoes a detectable change when the first oligonucleotides hybridise to both a target polynucleotide and a second oligonucleotide; said second oligonucleotide having a first and second section, wherein the first section comprises a nucleotide sequence which is capable of hybridising to a nucleotide sequence of the first section of the first oligonucleotide; and the second section comprises a nucleotide sequence that is capable of hybridising to a nucleotide sequence of the target polynucleotide that is adjacent to the nucleotide sequence that the second section of the first oligonucleotide is capable of hybridising to, said second section of the first oligonucleotide having a melting temperature (Tm) of between 25 and 50° C. and the second section of the second oligonucleotide having a melting temperature (Tm) of between 40 and 70° C.

66. The oligonucleotide library of claim 65 in which the plurality of oligonucleotides may each comprise a different visually detectable label.

67. The oligonucleotide library of claim 65 or 66 wherein the first oligonucleotide is labelled internally with the at least one visually detectable label.

68. The oligonucleotide library of any of claims 65 to 67 wherein the solid support is a polynucleotide synthesis resin.

69. The oligonucleotide library of any of claims 65 to 68 wherein the visually detectable label is a fluorophore or a dye.

70. The oligonucleotide library of any of claims 65 to 69 wherein the visually detectable label is fluorescein dT.

71. The oligonucleotide library of any of claims 65 to 70 wherein the first section of the first oligonucleotide is labelled with two or more; three or more; or four or more visually detectable labels.

72. A method of using the oligonucleotide library of any of claims 65 to 71 to produce an oligonucleotide probe, comprising the steps of extending the first section of the first oligonucleotide of at least one of the oligonucleotides making up the oligonucleotide library.

73. The method of claim 72 wherein the extension of the first section of the first oligonucleotide is by joining a second oligonucleotide section to the first section by means of ‘click chemistry’.

74. The oligonucleotide hybridisation structure, use, kit of parts, oligonucleotide library or method of any of claims 51 to 73 wherein the first and second sections of the first oligonucleotide are joined at a junction, said junction being positioned at least two nucleotides from the visually detectable label positioned 5′-most when the first oligonucleotide has been synthesised in the 3′ to 5′ direction; or said junction being positioned at least two nucleotides from the visually detectable label positioned 3′-most when the first oligonucleotide has been synthesised in the 5′ to 3′ direction.

75. The oligonucleotide hybridisation structure, use, kit of parts, oligonucleotide library or method of claim 74 wherein the junction is positioned at least three nucleotides from the visually detectable label positioned 5′-most when the first oligonucleotide has been synthesised in the 3′ to 5′ direction; or said junction being positioned at least three nucleotides from the visually detectable label positioned 3′-most when the first oligonucleotide has been synthesised in the 5′ to 3′ direction.

76. The oligonucleotide hybridisation structure, use, kit of parts, oligonucleotide library or method of claim 74 or 75 wherein the junction is positioned at least four nucleotides from the visually detectable label positioned 5′-most when the first oligonucleotide has been synthesised in the 3′ to 5′ direction; or said junction being positioned at least four nucleotides from the visually detectable label positioned 3′-most when the first oligonucleotide has been synthesised in the 5′ to 3′ direction.

77. The oligonucleotide hybridisation structure, use, kit of parts, oligonucleotide library or method of any of claims 51 to 76 the junction is positioned two, three or four nucleotides from the visually detectable label positioned 5′-most when the first oligonucleotide has been synthesised in the 3′ to 5′ direction; or said junction being positioned at least two, three or four nucleotides from the visually detectable label positioned 3′-most when the first oligonucleotide has been synthesised in the 5′ to 3′ direction.

78. The oligonucleotide hybridisation structure, use, kit of parts, oligonucleotide library or method of any of claims 51 to 73 wherein the first and second sections of the first oligonucleotide are joined at a junction, said junction being positioned one nucleotide from the visually detectable label positioned 5′-most when the first oligonucleotide has been synthesised in the 3′ to 5′ direction; or said junction being positioned at one nucleotide from the visually detectable label positioned 3′-most when the first oligonucleotide has been synthesised in the 5′ to 3′ direction.

79. The oligonucleotide hybridisation structure, use, kit of parts, oligonucleotide library or method of any of claims 51 to 78 wherein the independent partner oligonucleotides are not labelled with a quencher.

80. A method of detecting the presence of a target polynucleotide and/or sequence variations within the target polynucleotide in a sample of interest substantially as described herein with reference to the figures and examples.

81. A probe system substantially as described herein with reference to the figures and examples.

82. An oligonucleotide hybridisation structure substantially as described herein with reference to the figures and examples.

83. A use of a probe system substantially as described herein with reference to the figures and examples.

84. An oligonucleotide library system substantially as described herein with reference to the figures and examples.


SEQUENCE LISTING (158 sequences)

SEQ ID NO: 1 - DNA, 27 bp, Artificial Sequence
  source 1..27: /mol_type=“unassigned DNA”/note=“Synthetic construct HRH2-JP ” /organism=“Artificial Sequence”
aaagtttgtcagccaccgccttagagt

SEQ ID NO: 2 - DNA, 28 bp, Artificial Sequence
  source 1..28: /mol_type=“unassigned DNA”/note=“Synthetic construct HRH2-UC ” /organism=“Artificial Sequence”
ccattcgcagacctcgctgacaaacttt

SEQ ID NO: 3 - DNA, 30 bp, Artificial Sequence
  source 1..30: /mol_type=“unassigned DNA”/note=“Synthetic construct HRH2-T1 ” /organism=“Artificial Sequence”
ctctaaggcggtggcgaggtctgcgaatgg

SEQ ID NO: 4 - DNA, 30 bp, Artificial Sequence
  source 1..30: /mol_type=“unassigned DNA”/note=“Synthetic construct HRH2-T2 ” /organism=“Artificial Sequence”
ctctaaggcgatggcgaggtctgcgaatgg

SEQ ID NO: 5 - DNA, 26 bp, Artificial Sequence
  source 1..26: /mol_type=“unassigned DNA” /note=“Synthetic construct CDK-JP ” /organism=“Artificial Sequence”
atttcaattatgtcccaacactcagt

SEQ ID NO: 6 - DNA, 27 bp, Artificial Sequence
  source 1..27: /mol_type=“unassigned DNA”/note=“Synthetic construct CDK-UC ” /organism=“Artificial Sequence”
ccattcgcagacctcgataattgaaat

SEQ ID NO: 7 - DNA, 30 bp, Artificial Sequence
  source 1..30: /mol_type=“unassigned DNA”/note=“Sytnthetic construct CDK-T1 ” /organism=“Artificial Sequence”
ctgagtgttgggaccgaggtctgcgaatgg

SEQ ID NO: 8 - DNA, 30 bp, Artificial Sequence
  source 1..30: /mol_type=“unassigned DNA” /note=“Synthetic construct CDK-T2 ” /organism=“Artificial Sequence”
ctgagtgttgagaccgaggtctgcgaatgg

SEQ ID NO: 9 - DNA, 35 bp, Artificial Sequence
  source 1..35: /mol_type=“unassigned DNA” /note=“Sytnthetic construct AIH7-JP ” /organism=“Artificial Sequence”
tcgcgtgtgaggagaggcctatttggtgctatagc

SEQ ID NO: 10 - DNA, 27 bp, Artificial Sequence
  source 1..27: /mol_type=“unassigned DNA”/note=“Synthetic construct AIH7-UC ” /organism=“Artificial Sequence”
agcaccaaatacgaggtctgcgaatgg

SEQ ID NO: 11 - DNA, 35 bp, Artificial Sequence
  source 1..35: /mol_type=“unassigned DNA”/note=“Synthetic construct AIH7-T1 ” /organism=“Artificial Sequence”
ccattcgcagacctcgggcctctcctcacacgcga

SEQ ID NO: 12 - DNA, 35 bp, Artificial Sequence
  source 1..35: /mol_type=“unassigned DNA”/note=“Synthetic construct AIH7-T2 ” /organism=“Artificial Sequence”
ccattcgcagacctcggacctctcctcacacacga

SEQ ID NO: 13 - DNA, 24 bp, Artificial Sequence
  source 1..24: /mol_type=“unassigned DNA” /note=“Synthetic construct CXC-JP ” /organism=“Artificial Sequence”
gtggtaggattgagcgagtcaggt

SEQ ID NO: 14 - DNA, 27 bp, Artificial Sequence
  source 1..27: /mol_type=“unassigned DNA”/note=“Synthetic construct CXC-UC ” /organism=“Artificial Sequence”
ccattcgcagacctcgaatcctaccac

SEQ ID NO: 15 - DNA, 28 bp, Artificial Sequence
  source 1..28: /mol_type=“unassigned DNA”/note=“Synthetic construct CXC-T1 ” /organism=“Artificial Sequence”
cctgactcgctccgaggtctgcgaatgg

SEQ ID NO: 16 - DNA, 28 bp, Artificial Sequence
  source 1..28: /mol_type=“unassigned DNA”/note=“Synthetic construct CXC-T2 ” /organism=“Artificial Sequence”
cctgactcactccgaggtctgcgaatgg

SEQ ID NO: 17 - DNA, 29 bp, Artificial Sequence
  source 1..29: /mol_type=“unassigned DNA”/note=“Synthetic construct CXC-T3” /organism=“Artificial Sequence”
cctgactcgctcccgaggtctgcgaatgg

SEQ ID NO: 18 - DNA, 25 bp, Artificial Sequence
  source 1..25: /mol_type=“unassigned DNA”/note=“Synthetic construct G1-JP ” /organism=“Artificial Sequence”
ccataacagcaaggttaggatggtg

SEQ ID NO: 19 - DNA, 33 bp, Artificial Sequence
  source 1..33: /mol_type=“unassigned DNA”/note=“Synthetic construct G1-UC ” /organism=“Artificial Sequence”
ccatcctaacagcttcgaaaaagttttctttac

SEQ ID NO: 20 - DNA, 36 bp, Artificial Sequence
  source 1..36: /mol_type=“unassigned DNA”/note=“Synthetic construct G1-T1 ” /organism=“Artificial Sequence”
gtaaagaaaactttttcgaagctcttgctgttatgg

SEQ ID NO: 21 - DNA, 36 bp, Artificial Sequence
  source 1..36: /mol_type=“unassigned DNA”/note=“Synthetic construct G1-T2 ” /organism=“Artificial Sequence”
gtaaagaaaactttttcgaagctcttgctattatgg

SEQ ID NO: 22 - DNA, 23 bp, Artificial Sequence
  source 1..23: /mol_type=“unassigned DNA”/note=“Synthetic construct P2-JP ” /organism=“Artificial Sequence”
ggggatcgattgttaggatggtg

SEQ ID NO: 23 - DNA, 32 bp, Artificial Sequence
  source 1..32: /mol_type=“unassigned DNA” /note=“Synthetic construct P2-UC ” /organism=“Artificial Sequence”
ccatcctaacgaaactctttttgtagagtttg

SEQ ID NO: 24 - DNA, 33 bp, Artificial Sequence
  source 1..33: /mol_type=“unassigned DNA”/note=“Synthetic construct P2-T1 ” /organism=“Artificial Sequence”
caaactctacaaaaagagtttcaatcgatcccc

SEQ ID NO: 25 - DNA, 33 bp, Artificial Sequence
  source 1..33: /mol_type=“unassigned DNA”/note=“Synthetic construct P2-T2 ” /organism=“Artificial Sequence”
caaactctacaaaaagagtttcaatcaatcccc

SEQ ID NO: 26 - DNA, 25 bp, Artificial Sequence
  source 1..25: /mol_type=“unassigned DNA”/note=“Synthetic construct CDK2-JP ” /organism=“Artificial Sequence”
ttatgtcccaacagttaggatggtg

SEQ ID NO: 27 - DNA, 31 bp, Artificial Sequence
  source 1..31: /mol_type=“unassigned DNA”/note=“Synthetic construct CDK2-UC ” /organism=“Artificial Sequence”
ccatcctaacctcagcttagatttgacccac

SEQ ID NO: 28 - DNA, 34 bp, Artificial Sequence
  source 1..34: /mol_type=“unassigned DNA”/note=“Synthetic construct CDK2-T1 ” /organism=“Artificial Sequence”
gtgggtcaaatctaagctgagtgttgggacataa

SEQ ID NO: 29 - DNA, 34 bp, Artificial Sequence
  source 1..34: /mol_type=“unassigned DNA”/note=“Synthetic construct CDK-T2 ” /organism=“Artificial Sequence”
gtgggtcaaatctaagctgagtgttgagacataa

SEQ ID NO: 30 - DNA, 25 bp, Artificial Sequence
  source 1..25: /mol_type=“unassigned DNA”/note=“Synthetic construct CH-JP ” /organism=“Artificial Sequence”
gtaatcaagcctggttaggatggtg

SEQ ID NO: 31 - DNA, 36 bp, Artificial Sequence
  source 1..36: /mol_type=“unassigned DNA”/note=“Synthetic construct CH-UC ” /organism=“Artificial Sequence”
ccatcctaaccaaatgtataccaagaaataaaaatg

SEQ ID NO: 32 - DNA, 39 bp, Artificial Sequence
  source 1..39: /mol_type=“unassigned DNA”/note=“Synthetic construct CH-T1 ” /organism=“Artificial Sequence”
catttttatttcttggtatacatttgcaggcttgattac

SEQ ID NO: 33 - DNA, 39 bp, Artificial Sequence
  source 1..39: /mol_type=“unassigned DNA”/note=“Synthetic construct CH-T2 ” /organism=“Artificial Sequence”
catttttatttcttggtatacatttgcaggcttaattac

SEQ ID NO: 34 - DNA, 27 bp, Artificial Sequence
  source 1..27: /mol_type=“unassigned DNA”/note=“Synthetic construct G1-JPv2 ” /organism=“Artificial Sequence”
ccataacagcaagtagttaggatggtg

SEQ ID NO: 35 - DNA, 34 bp, Artificial Sequence
  source 1..34: /mol_type=“unassigned DNA”/note=“Synthetic construct G1-UCv2 ” /organism=“Artificial Sequence”
catcctaactaagcttcgaaaaagttttctttac

SEQ ID NO: 36 - DNA, 24 bp, Artificial Sequence
  source 1..24: /mol_type=“unassigned DNA”/note=“Synthetic construct G1-JPv2RC ” /organism=“Artificial Sequence”
catcctaactacttgctgttatgg

SEQ ID NO: 37 - DNA, 24 bp, Artificial Sequence
  source 1..24: /mol_type=“unassigned DNA”/note=“Synthetic construct G1-JPv2RC2 ” /organism=“Artificial Sequence”
catcctaactacttgctattatgg

SEQ ID NO: 38 - DNA, 25 bp, Artificial Sequence
  source 1..25: /mol_type=“unassigned DNA”/note=“Synthetic construct P2-JPv2 ” /organism=“Artificial Sequence”
ggggatcgatttagttaggatggtg

SEQ ID NO: 39 - DNA, 33 bp, Artificial Sequence
  source 1..33: /mol_type=“unassigned DNA”/note=“Synthetic construct P2-UCv2 ” /organism=“Artificial Sequence”
catcctaactagaaactctttttgtagagtttg

SEQ ID NO: 40 - DNA, 25 bp, Artificial Sequence
  source 1..25: /mol_type=“unassigned DNA”/note=“Synthetic construct G1-L ” /organism=“Artificial Sequence”
cttcacgttccataacagcaagagt

SEQ ID NO: 41 - DNA, 26 bp, Artificial Sequence
  source 1..26: /mol_type=“unassigned DNA”/note=“Synthetic construct G1-LF ” /organism=“Artificial Sequence”
cggagttcctagaggattcctagacg

SEQ ID NO: 42 - DNA, 25 bp, Artificial Sequence
  source 1..25: /mol_type=“unassigned DNA”/note=“Synthetic construct G1-LR ” /organism=“Artificial Sequence”
taccgcatgtttcaaaacactgaga

SEQ ID NO: 43 - DNA, 27 bp, Artificial Sequence
  source 1..27: /mol_type=“unassigned DNA”/note=“Synthetic construct P2-L ” /organism=“Artificial Sequence”
ggatcgattgaaactctttttgtagat

SEQ ID NO: 44 - DNA, 32 bp, Artificial Sequence
  source 1..32: /mol_type=“unassigned DNA”/note=“Synthetic construct P2-LF ” /organism=“Artificial Sequence”
catattacgagctttttataaacctccccaac

SEQ ID NO: 45 - DNA, 22 bp, Artificial Sequence
  source 1..22: /mol_type=“unassigned DNA”/note=“Synthetic construct 45 P2-LR ” /organism=“Artificial Sequence”
tcgccgatgagttcgacattcc

SEQ ID NO: 46 - DNA, 36 bp, Artificial Sequence
  source 1..36: /mol_type=“unassigned DNA”/note=“Synthetic construct G1-T3 ” /organism=“Artificial Sequence”
gtaaagaaaactttttcgaagctcttgctgttatag

SEQ ID NO: 47 - DNA, 36 bp, Artificial Sequence
  source 1..36: /mol_type=“unassigned DNA”/note=“Synthetic construct G1-T4 ” /organism=“Artificial Sequence”
gtaaagaaaactttttcgaagctattgctgttatag

SEQ ID NO: 48 - DNA, 36 bp, Artificial Sequence
  source 1..36: /mol_type=“unassigned DNA”/note=“Synthetic construct G1-T5 ” /organism=“Artificial Sequence”
gtaaagaaaactttttcgaagctattgctattatgg

SEQ ID NO: 49 - DNA, 36 bp, Artificial Sequence
  source 1..36: /mol_type=“unassigned DNA”/note=“Synthetic construct G1-T6 ” /organism=“Artificial Sequence”
gtaaagaaaactttttcaaagctcttgctgttatgg

SEQ ID NO: 50 - DNA, 36 bp, Artificial Sequence
  source 1..36: /mol_type=“unassigned DNA”/note=“Synthetic construct G1-T7 ” /organism=“Artificial Sequence”
gtaaataaaactttttcaaagctcttgctgttatgg

SEQ ID NO: 51 - DNA, 28 bp, Artificial Sequence
  source 1..28: /mol_type=“unassigned DNA”/note=“Synthetic construct G1-UCv3 ” /organism=“Artificial Sequence”
catcctaactaagcttcgaaaaagtttt

SEQ ID NO: 52 - DNA, 24 bp, Artificial Sequence
  source 1..24: /mol_type=“unassigned DNA”/note=“Synthetic construct G1-UCv4 ” /organism=“Artificial Sequence”
catcctaactaagcttcgaaaaag

SEQ ID NO: 53 - DNA, 27 bp, Artificial Sequence
  source 1..27: /mol_type=“unassigned DNA”/note=“Synthetic construct CH-JPv2-UE1 ” /organism=“Artificial Sequence”
gtaatcaagcctgtagttaggatggtg

SEQ ID NO: 54 - DNA, 27 bp, Artificial Sequence
  source 1..27: /mol_type=“unassigned DNA”/note=“Synthetic construct CH-JPv2-UE2 ” /organism=“Artificial Sequence”
gtaatcaagcctgtattggagatgtgc

SEQ ID NO: 55 - DNA, 29 bp, Artificial Sequence
  source 1..29: /mol_type=“unassigned DNA”/note=“Synthetic construct CH-UCv2-UE1 ” /organism=“Artificial Sequence”
caccatcctaactacaaatgtataccaag

SEQ ID NO: 56 - DNA, 29 bp, Artificial Sequence
  source 1..29: /mol_type=“unassigned DNA”/note=“Synthetic construct CH-UCv2-UE2 ” /organism=“Artificial Sequence”
gcacatctccaatacaaatgtataccaag

SEQ ID NO: 57 - DNA, 27 bp, Artificial Sequence
  source 1..27: /mol_type=“unassigned DNA”/note=“Synthetic construct CH-JPv2-SIMA ” /organism=“Artificial Sequence”
gtaatcaagcctgtagttaggatggtg

SEQ ID NO: 58 - DNA, 30 bp, Artificial Sequence
  source 1..30: /mol_type=“unassigned DNA”/note=“Synthetic construct JP-22 ” /organism=“Artificial Sequence”
ctttgtaatcaagcctgtattatgatattg

SEQ ID NO: 59 - DNA, 36 bp, Artificial Sequence
  source 1..36: /mol_type=“unassigned DNA”/note=“Synthetic construct UC-22 ” /organism=“Artificial Sequence”
caatatcataatacgcaaatgtataccaagaaataa

SEQ ID NO: 60 - DNA, 36 bp, Artificial Sequence
  source 1..36: /mol_type=“unassigned DNA”/note=“Synthetic construct T-22 ” /organism=“Artificial Sequence”
ggatgtacaacaatatctgttagtcacctttgggtc

SEQ ID NO: 61 - DNA, 37 bp, Artificial Sequence
  source 1..37: /mol_type=“unassigned DNA”/note=“Synthetic construct CH-UC4v2 ” /organism=“Artificial Sequence”
catcctaactacaaatgtataccaagaaataaaaatg

SEQ ID NO: 62 - DNA, 30 bp, Artificial Sequence
  source 1..30: /mol_type=“unassigned DNA”/note=“Synthetic construct CH-F ” /organism=“Artificial Sequence”
gggttcgttgtagagccatgtcctatcttg

SEQ ID NO: 63 - DNA, 30 bp, Artificial Sequence
  source 1..30: /mol_type=“unassigned DNA”/note=“Synthetic construct CH-R ” /organism=“Artificial Sequence”
cgcagctgctgtaatcacccagtcgataaa

SEQ ID NO: 64 - DNA, 31 bp, Artificial Sequence
  source 1..31: /mol_type=“unassigned DNA”/note=“Synthetic construct S1-JP ” /organism=“Artificial Sequence”
ccaccgtatggctaagggtattatgatattg

SEQ ID NO: 65 - DNA, 34 bp, Artificial Sequence
  source 1..34: /mol_type=“unassigned DNA”/note=“Synthetic construct S1-UC ” /organism=“Artificial Sequence”
caatatcataatacagattcgctttaagaaggct

SEQ ID NO: 66 - DNA, 24 bp, Artificial Sequence
  source 1..24: /mol_type=“unassigned DNA”/note=“Synthetic construct S1-F ” /organism=“Artificial Sequence”
gggctttgttcacctctttcaacg

SEQ ID NO: 67 - DNA, 22 bp, Artificial Sequence
  source 1..22: /mol_type=“unassigned DNA”/note=“Synthetic construct S1-R ” /organism=“Artificial Sequence”
cctctgcgtaacttctcttccg

SEQ ID NO: 68 - DNA, 28 bp, Artificial Sequence
  source 1..28: /mol_type=“unassigned DNA”/note=“Synthetic construct JP-1 ” /organism=“Artificial Sequence”
ggatctggatgatggtattatgatattg

SEQ ID NO: 69 - DNA, 27 bp, Artificial Sequence
  source 1..27: /mol_type=“unassigned DNA”/note=“Synthetic construct UC-1 ” /organism=“Artificial Sequence”
caatatcataatacggcacaggcgggc

SEQ ID NO: 70 - DNA, 27 bp, Artificial Sequence
  source 1..27: /mol_type=“unassigned DNA”/note=“Synthetic construct T-1.1 ” /organism=“Artificial Sequence”
gcccgcctgtgcccatcatccagatcc

SEQ ID NO: 71 - DNA, 27 bp, Artificial Sequence
  source 1..27: /mol_type=“unassigned DNA”/note=“Synthetic construct T-1.2 ” /organism=“Artificial Sequence”
gcccgcctgtgcccatcacccagatcc

SEQ ID NO: 72 - DNA, 29 bp, Artificial Sequence
  source 1..29: /mol_type=“unassigned DNA”/note=“Synthetic construct JP-2 ” /organism=“Artificial Sequence”
cgcttctccgtctccgtattatgatattg

SEQ ID NO: 73 - DNA, 31 bp, Artificial Sequence
  source 1..31: /mol_type=“unassigned DNA”/note=“Synthetic construct UC-2 ” /organism=“Artificial Sequence”
caatatcataatacaccttgcgcaacttggg

SEQ ID NO: 74 - DNA, 32 bp, Artificial Sequence
  source 1..32: /mol_type=“unassigned DNA”/note=“Synthetic construct T-2.1 ” /organism=“Artificial Sequence”
cccaagttgcgcaaggtggagacggagaagcg

SEQ ID NO: 75 - DNA, 32 bp, Artificial Sequence
  source 1..32: /mol_type=“unassigned DNA”/note=“Synthetic construct T-2.2 ” /organism=“Artificial Sequence”
cccaagttgcgcaaggtggagatggagaagcg

SEQ ID NO: 76 - DNA, 32 bp, Artificial Sequence
  source 1..32: /mol_type=“unassigned DNA”/note=“Synthetic construct T-2.3 ” /organism=“Artificial Sequence”
cccaagttgcgcaaggtggacacggagaagcg

SEQ ID NO: 77 - DNA, 26 bp, Artificial Sequence
  source 1..26: /mol_type=“unassigned DNA”/note=“Synthetic construct JP-3 ” /organism=“Artificial Sequence”
tacatccggatggtattatgatattg

SEQ ID NO: 78 - DNA, 29 bp, Artificial Sequence
  source 1..29: /mol_type=“unassigned DNA”/note=“Synthetic construct UC-3 ” /organism=“Artificial Sequence”
caatatcataatactgcagcgtgagccca

SEQ ID NO: 79 - DNA, 27 bp, Artificial Sequence
  source 1..27: /mol_type=“unassigned DNA”/note=“Synthetic construct T-3.1 ” /organism=“Artificial Sequence”
tgggctcacgctgcacatccggatgta

SEQ ID NO: 80 - DNA, 27 bp, Artificial Sequence
  source 1..27: /mol_type=“unassigned DNA”/note=“Synthetic construct T-3.2 ” /organism=“Artificial Sequence”
tgggctcacgctgcacatccggaggta

SEQ ID NO: 81 - DNA, 24 bp, Artificial Sequence
  source 1..24: /mol_type=“unassigned DNA”/note=“Synthetic construct JP-4 ” /organism=“Artificial Sequence”
gcgtcctggggtattatgatattg

SEQ ID NO: 82 - DNA, 29 bp, Artificial Sequence
  source 1..29: /mol_type=“unassigned DNA”/note=“Synthetic construct UC-4 ” /organism=“Artificial Sequence”
caatatcataatacggtgggagatgcggg

SEQ ID NO: 83 - DNA, 25 bp, Artificial Sequence
  source 1..25: /mol_type=“unassigned DNA”/note=“Synthetic construct T-4.1 ” /organism=“Artificial Sequence”
cccgcatctcccacccccaggacgc

SEQ ID NO: 84 - DNA, 25 bp, Artificial Sequence
  source 1..25: /mol_type=“unassigned DNA”/note=“Synthetic construct T-4.2 ” /organism=“Artificial Sequence”
cccgcatctcccacccccagaacgc

SEQ ID NO: 85 - DNA, 27 bp, Artificial Sequence
  source 1..27: /mol_type=“unassigned DNA”/note=“Synthetic construct JP-5 ” /organism=“Artificial Sequence”
tcatcctgtgctcgtattatgatattg

SEQ ID NO: 86 - DNA, 33 bp, Artificial Sequence
  source 1..33: /mol_type=“unassigned DNA”/note=“Synthetic construct UC-5 ” /organism=“Artificial Sequence”
caatatcataatacagttagcagctcatccagc

SEQ ID NO: 87 - DNA, 32 bp, Artificial Sequence
  source 1..32: /mol_type=“unassigned DNA”/note=“Synthetic construct T-5.1 ” /organism=“Artificial Sequence”
gctggatgagctgctaactgagcacaggatga

SEQ ID NO: 88 - DNA, 31 bp, Artificial Sequence
  source 1..31: /mol_type=“unassigned DNA”/note=“Synthetic construct T-5.2 ” /organism=“Artificial Sequence”
gctggatgagctgctaactgagcacggatga

SEQ ID NO: 89 - DNA, 28 bp, Artificial Sequence
  source 1..28: /mol_type=“unassigned DNA”/note=“Synthetic construct JP-6 ” /organism=“Artificial Sequence”
gatggagaaggtgagtattatgatattg

SEQ ID NO: 90 - DNA, 30 bp, Artificial Sequence
  source 1..30: /mol_type=“unassigned DNA”/note=“Synthetic construct UC-6 ” /organism=“Artificial Sequence”
caatatcataatacgagtggctgccacggt

SEQ ID NO: 91 - DNA, 30 bp, Artificial Sequence
  source 1..30: /mol_type=“unassigned DNA”/note=“Synthetic construct labelled T-6.1 ” /organism=“Artificial Sequence”
accgtggcagccactctcaccttctccatc

SEQ ID NO: 92 - DNA, 27 bp, Artificial Sequence
  source 1..27: /mol_type=“unassigned DNA”/note=“Synthetic construct T-6.2 ” /organism=“Artificial Sequence”
accgtggcagccactctcacctccatc

SEQ ID NO: 93 - DNA, 28 bp, Artificial Sequence
  source 1..28: /mol_type=“unassigned DNA”/note=“Synthetic construct JP-7 ” /organism=“Artificial Sequence”
atgagaacctgtgcgtattatgatattg

SEQ ID NO: 94 - DNA, 31 bp, Artificial Sequence
  source 1..31: /mol_type=“unassigned DNA”/note=“Synthetic construct UC-7 ” /organism=“Artificial Sequence”
caatatcataatactagtggtggctgacctg

SEQ ID NO: 95 - DNA, 31 bp, Artificial Sequence
  source 1..31: /mol_type=“unassigned DNA”/note=“Synthetic construct T-7.1 ” /organism=“Artificial Sequence”
caggtcagccaccactagcacaggttctcat

SEQ ID NO: 96 - DNA, 31 bp, Artificial Sequence
  source 1..31: /mol_type=“unassigned DNA”/note=“Synthetic construct T-7.2 ” /organism=“Artificial Sequence”
caggtcagccaccactagcgcaggttctcat

SEQ ID NO: 97 - DNA, 25 bp, Artificial Sequence
  source 1..25: /mol_type=“unassigned DNA”/note=“Synthetic construct JP-8 ” /organism=“Artificial Sequence”
cccttcctcccgtattatgatattg

SEQ ID NO: 98 - DNA, 27 bp, Artificial Sequence
  source 1..27: /mol_type=“unassigned DNA”/note=“Synthetic construct UC-8 ” /organism=“Artificial Sequence”
caatatcataatactcggcccctgcac

SEQ ID NO: 99 - DNA, 24 bp, Artificial Sequence
  source 1..24: /mol_type=“unassigned DNA”/note=“Synthetic construct T-8.1 ” /organism=“Artificial Sequence”
gtgcaggggccgagggaggaaggg

SEQ ID NO: 100 - DNA, 24 bp, Artificial Sequence
  source 1..24: /mol_type=“unassigned DNA”/note=“Synthetic construct T-8.2 ” /organism=“Artificial Sequence”
gtgcaggggccgagggagaaaggg

SEQ ID NO: 101 - DNA, 27 bp, Artificial Sequence
  source 1..27: /mol_type=“unassigned DNA”/note=“Synthetic construct CH-JPv2-UE1np ” /organism=“Artificial Sequence”
gtaatcaagcctgtagttaggatggtg

SEQ ID NO: 102 - DNA, 41 bp, Artificial Sequence
  source 1..41: /mol_type=“unassigned DNA”/note=“Synthetic construct S1-FIP ” /organism=“Artificial Sequence”
ttgcagctccttctcttgttccgtggagcaagccttcttaa

SEQ ID NO: 103 - DNA, 44 bp, Artificial Sequence
  source 1..44: /mol_type=“unassigned DNA”/note=“Synthetic construct S1-BIP ” /organism=“Artificial Sequence”
gcctattgtagtgcggaagagaatcggtctaagcttcctaattc

SEQ ID NO: 104 - DNA, 19 bp, Artificial Sequence
  source 1..19: /mol_type=“unassigned DNA”/note=“Synthetic construct S1-F3 ” /organism=“Artificial Sequence”
gttcacctctttcaacggt

SEQ ID NO: 105 - DNA, 19 bp, Artificial Sequence
  source 1..19: /mol_type=“unassigned DNA”/note=“Synthetic construct S1-B3 ” /organism=“Artificial Sequence”
tcgtgttctccaagagtct

SEQ ID NO: 106 - DNA, 20 bp, Artificial Sequence
  source 1..20: /mol_type=“unassigned DNA”/note=“Synthetic construct S1-LB ” /organism=“Artificial Sequence”
aggctgtggagcagttaatc

SEQ ID NO: 107 - DNA, 34 bp, Artificial Sequence
  source 1..34: /mol_type=“unassigned DNA” /note=“Synthetic construct G1-JPrc ” /organism=“Artificial Sequence”
ccatcctaacagcttcgaaaaagtttttctttac

SEQ ID NO: 108 - DNA, 25 bp, Artificial Sequence
  source 1..25: /mol_type=“unassigned DNA”/note=“Synthetic construct G1-UCrc ” /organism=“Artificial Sequence”
ccataacagcaaggttaggatggtg

SEQ ID NO: 109 - DNA, 24 bp, Artificial Sequence
  source 1..24: /mol_type=“unassigned DNA”/note=“Synthetic construct CRUX-UC ” /organism=“Artificial Sequence”
ccataacagcaagaatcgatcccc

SEQ ID NO: 110 - DNA, 31 bp, Artificial Sequence
  source 1..31: /mol_type=“unassigned DNA”/note=“Synthetic construct 1661-UC1 ” /organism=“Artificial Sequence”
caccatcctaactaaccttgcgcaacttggg

SEQ ID NO: 111 - DNA, 23 bp, Artificial Sequence
  source 1..23: /mol_type=“unassigned DNA”/note=“Synthetic construct 1661-UC2 ” /organism=“Artificial Sequence”
cttctccgtctccaatcgatccc

SEQ ID NO: 112 - DNA, 20 bp, Artificial Sequence
  source 1..20: /mol_type=“unassigned DNA”/note=“Synthetic construct 1661-F ” /organism=“Artificial Sequence”
gggagaccagggggagcata

SEQ ID NO: 113 - DNA, 19 bp, Artificial Sequence
  source 1..19: /mol_type=“unassigned DNA”/note=“Synthetic construct 1661-R ” /organism=“Artificial Sequence”
tgcccatcacccaccggag

SEQ ID NO: 114 - DNA, 37 bp, Artificial Sequence
  source 1..37: /mol_type=“unassigned DNA”/note=“Synthetic construct G1-1nt ” /organism=“Artificial Sequence”
gtaaagaaaactttttcgaagctacttgctgttatgg

SEQ ID NO: 115 - DNA, 38 bp, Artificial Sequence
  source 1..38: /mol_type=“unassigned DNA”/note=“Synthetic construct G1-2nt ” /organism=“Artificial Sequence”
gtaaagaaaactttttcgaagctaacttgctgttatgg

SEQ ID NO: 116 - DNA, 39 bp, Artificial Sequence
  source 1..39: /mol_type=“unassigned DNA”/note=“Synthetic construct G1-3nt ” /organism=“Artificial Sequence”
gtaaagaaaactttttcgaagctaaacttgctgttatgg

SEQ ID NO: 117 - DNA, 41 bp, Artificial Sequence
  source 1..41: /mol_type=“unassigned DNA”/note=“Synthetic construct G1-5nt ” /organism=“Artificial Sequence”
gtaaagaaaactttttcgaagctaaaaacttgctgttatgg

SEQ ID NO: 118 - DNA, 46 bp, Artificial Sequence
  source 1..46: /mol_type=“unassigned DNA”/note=“Synthetic construct G1-10nt ” /organism=“Artificial Sequence”
gtaaagaaaactttttcgaagctaaaaaaaaaacttgctgttatgg

SEQ ID NO: 119 - DNA, 56 bp, Artificial Sequence
  source 1..56: /mol_type=“unassigned DNA”/note=“Synthetic construct G1-20nt ” /organism=“Artificial Sequence”
gtaaagaaaactttttcgaagctaaaaaaaaaaaaaaaaaaaacttgctgttatgg

SEQ ID NO: 120 - DNA, 44 bp, Artificial Sequence
  source 1..44: /mol_type=“unassigned DNA”/note=“Synthetic construct D16-BL-UC ” /organism=“Artificial Sequence”
catcctaactatatctatctatctatctatctatcacctgtctg

SEQ ID NO: 121 - DNA, 41 bp, Artificial Sequence
  source 1..41: /mol_type=“unassigned DNA”/note=“Synthetic construct D16-P-UC ” /organism=“Artificial Sequence”
caatgatatctatctatctatctatctatcaatcgatcccc

SEQ ID NO: 122 - DNA, 56 bp, Artificial Sequence
  source 1..56: /mol_type=“unassigned DNA”/note=“Synthetic construct D16-10rpt ” /organism=“Artificial Sequence”
cagacaggtggatagatagatagatagatagatagatagatagatagatatcattg

SEQ ID NO: 123 - DNA, 60 bp, Artificial Sequence
  source 1..60: /mol_type=“unassigned DNA”/note=“Synthetic construct D16-11rpt ” /organism=“Artificial Sequence”
cagacaggtggatagatagatagatagatagatagatagatagatagatagatatcattg

SEQ ID NO: 124 - DNA, 64 bp, Artificial Sequence
  source 1..64: /mol_type=“unassigned DNA”/note=“Synthetic construct D16-12rpt ” /organism=“Artificial Sequence”
cagacaggtggatagatagatagatagatagatagatagatagatagatagatagatatcattg

SEQ ID NO: 125 - DNA, 68 bp, Artificial Sequence
  source 1..68: /mol_type=“unassigned DNA”/note=“Synthetic construct D16-13rpt ” /organism=“Artificial Sequence”
cagacaggtggatagatagatagatagatagatagatagatagatagatagatagatagatatcattg

SEQ ID NO: 126 - DNA, 25 bp, Artificial Sequence
  source 1..25: /mol_type=“unassigned DNA”/note=“Synthetic construct P2-5JP ” /organism=“Artificial Sequence”
ggggatcgatttagttaggatggtg

SEQ ID NO: 127 - DNA, 25 bp, Artificial Sequence
  source 1..25: /mol_type=“unassigned DNA”/note=“Synthetic construct P2-3JP ” /organism=“Artificial Sequence”
ggggatcgatttagttaggatggtg

SEQ ID NO: 128 - DNA, 37 bp, Artificial Sequence
  source 1..37: /mol_type=“unassigned DNA”/note=“Synthetic construct G1-UCv3 ” /organism=“Artificial Sequence”
caccatcctaactaagcttcgaaaaagttttctttac

SEQ ID NO: 129 - DNA, 40 bp, Artificial Sequence
  source 1..40: /mol_type=“unassigned DNA”/note=“Synthetic construct G1-UCv4 ” /organism=“Artificial Sequence”
aaacaccatcctaactaagcttcgaaaaagttttctttac

SEQ ID NO: 130 - DNA, 27 bp, Artificial Sequence
  source 1..27: /mol_type=“unassigned DNA”/note=“Synthetic construct CRUX-UCv2 ” /organism=“Artificial Sequence”
ccataacagcaagaatcgatccccaaa

SEQ ID NO: 131 - DNA, 36 bp, Artificial Sequence
  source 1..36: /mol_type=“unassigned DNA”/note=“Synthetic construct P2-UCv3 ” /organism=“Artificial Sequence”
caccatcctaactagaaactctttttgtagagtttg

SEQ ID NO: 132 - DNA, 39 bp, Artificial Sequence
  source 1..39: /mol_type=“unassigned DNA”/note=“Synthetic construct P2-UCv4 ” /organism=“Artificial Sequence”
aaacaccatcctaactagaaactctttttgtagagtttg

SEQ ID NO: 133 - DNA, 12 bp, Artificial Sequence
  source 1..12: /mol_type=“unassigned DNA”/note=“Silver Nanocluster attachment preferred motif ” /organism=“Artificial Sequence”
cccwwwwwcccc

SEQ ID NO: 134 - DNA, 12 bp, Artificial Sequence
  source 1..12: /mol_type=“unassigned DNA”/note=“HRH2-JP universal element ” /organism=“Artificial Sequence”
aaagtttgtcag

SEQ ID NO: 135 - DNA, 14 bp, Artificial Sequence
  source 1..14: /mol_type=“unassigned DNA”/note=“HRH2-JP target-specific sequence ” /organism=“Artificial Sequence”
ccaccgccttagag

SEQ ID NO: 136 - DNA, 16 bp, Artificial Sequence
  source 1..16: /mol_type=“unassigned DNA”/note=“HRH2-UC target-specific sequence ” /organism=“Artificial Sequence”
ccattcgcagacctcg

SEQ ID NO: 137 - DNA, 12 bp, Artificial Sequence
  source 1..12: /mol_type=“unassigned DNA”/note=“HRH2-UC universal element ” /organism=“Artificial Sequence”
ctgacaaacttt

SEQ ID NO: 138 - DNA, 11 bp, Artificial Sequence
  source 1..11: /mol_type=“unassigned DNA”/note=“CDK-JP universal element” /organism=“Artificial Sequence”
atttcaattat

SEQ ID NO: 139 - DNA, 14 bp, Artificial Sequence
  source 1..14: /mol_type=“unassigned DNA”/note=“CDK-JP target-specific sequence ” /organism=“Artificial Sequence”
gtcccaacactcag

SEQ ID NO: 140 - DNA, 11 bp, Artificial Sequence
  source 1..11: /mol_type=“unassigned DNA”/note=“CDK-UC universal element” /organism=“Artificial Sequence”
ataattgaaat

SEQ ID NO: 141 - DNA, 19 bp, Artificial Sequence
  source 1..19: /mol_type=“unassigned DNA”/note=“AIH7-JP target-specific sequence ” /organism=“Artificial Sequence”
tcgcgtgtgaggagaggcc

SEQ ID NO: 142 - DNA, 16 bp, Artificial Sequence
  source 1..16: /mol_type=“unassigned DNA”/note=“AIH7-JP universal element ” /organism=“Artificial Sequence”
tatttggtgctatagc

SEQ ID NO: 143 - DNA, 11 bp, Artificial Sequence
  source 1..11: /mol_type=“unassigned DNA”/note=“AIH7-UC universal element ” /organism=“Artificial Sequence”
agcaccaaata

SEQ ID NO: 144 - DNA, 16 bp, Artificial Sequence
  source 1..16: /mol_type=“unassigned DNA”/note=“AIH7-UC target-specific sequence ” /organism=“Artificial Sequence”
cgaggtctgcgaatgg

SEQ ID NO: 145 - DNA, 11 bp, Artificial Sequence
  source 1..11: /mol_type=“unassigned DNA”/note=“CXC-JP universal element ” /organism=“Artificial Sequence”
gtggtaggatt

SEQ ID NO: 146 - DNA, 12 bp, Artificial Sequence
  source 1..12: /mol_type=“unassigned DNA”/note=“CXC-JP target-specific sequence ” /organism=“Artificial Sequence”
gagcgagtcagg

SEQ ID NO: 147 - DNA, 11 bp, Artificial Sequence
  source 1..11: /mol_type=“unassigned DNA”/note=“CXC-UC universal element ” /organism=“Artificial Sequence”
aatcctaccac

SEQ ID NO: 148 - DNA, 12 bp, Artificial Sequence
  source 1..12: /mol_type=“unassigned DNA”/note=“G1- JP/P2-JP/CDK2-JP/CH-JP universal element ” /organism=“Artificial Sequence”
gttaggatggtg

SEQ ID NO: 149 - DNA, 10 bp, Artificial Sequence
  source 1..10: /mol_type=“unassigned DNA”/note=“G1- UC/P2-UC/CDK2-UC/CH-UC universal element ” /organism=“Artificial Sequence”
ccatcctaac

SEQ ID NO: 150 - DNA, 14 bp, Artificial Sequence
  source 1..14: /mol_type=“unassigned DNA”/note=“G1-JPv2, P2-JPv2 universal element ” /organism=“Artificial Sequence”
tagttaggatggtg

SEQ ID NO: 151 - DNA, 11 bp, Artificial Sequence
  source 1..11: /mol_type=“unassigned DNA”/note=“G1-UCv2, P2-UCv2 universal element ” /organism=“Artificial Sequence”
catcctaacta

SEQ ID NO: 152 - DNA, 14 bp, Artificial Sequence
  source 1..14: /mol_type=“unassigned DNA”/note=“CH-UCv2-UE1 universal element ” /organism=“Artificial Sequence”
caccatcctaacta

SEQ ID NO: 153 - DNA, 14 bp, Artificial Sequence
  source 1..14: /mol_type=“unassigned DNA”/note=“CH-JPv2-UE2 universal element” /organism=“Artificial Sequence”
tattggagatgtgc

SEQ ID NO: 154 - DNA, 14 bp, Artificial Sequence
  source 1..14: /mol_type=“unassigned DNA”/note=“CH-UCv2-UE2 universal element ” /organism=“Artificial Sequence”
gcacatctccaata

SEQ ID NO: 155 - DNA, 14 bp, Artificial Sequence
  source 1..14: /mol_type=“unassigned DNA”/note=“JP-22 universal element ” /organism=“Artificial Sequence”
gtattatgatattg

SEQ ID NO: 156 - DNA, 14 bp, Artificial Sequence
  source 1..14: /mol_type=“unassigned DNA”/note=“UC-22 universal element ” /organism=“Artificial Sequence”
caatatcataatac

SEQ ID NO: 157 - DNA, 10 bp, Artificial Sequence
  source 1..10: /mol_type=“unassigned DNA”/note=“G1-JPrc universal element ” /organism=“Artificial Sequence”
ccatcctaac

SEQ ID NO: 158 - DNA, 12 bp, Artificial Sequence
  source 1..12: /mol_type=“unassigned DNA”/note=“G1-UCrc universal element ” /organism=“Artificial Sequence”
gttaggatggtg